(19) United States
(12) Patent Application Publication
(10) Pub. No.: US 2024/0327840 A1
(43) Pub. Date: Oct. 3, 2024

(54) OLIGONUCLEOTIDE FOR INHIBITING QUAKING ACTIVITY

(71) Applicant: ACADEMISCH ZIEKENHUIS LEIDEN H.O.D.N. LUMC, Leiden (NL)

(72) Inventors: Anton Jan VAN ZONNEVELD, Soest (NL); Ruben Gosewinus DE BRUIN, The Hague (NL); Jurriën PRINS, The Hague (NL); Gerardus Theodorus Marie WAGENAAR, Amstelveen (NL); Falk WACHOWIUS, Groningen (NL); Eric Peter VAN DER VEER, The Hague (NL)

(21) Appl. No.: 18/579,115

(22) PCT Filed: Jul. 15, 2022
(86) PCT No.: PCT/EP2022/069965

§ 371 Date: Jan. 12, 2024

(30) Foreign Application Priority Data
Jul. 16, 2021 (EP) 21186214.9

(51) Int. Cl. C12N 15/113; A61P 29/00; C12N 15/86

(52) U.S. Cl. CPC C12N 15/113; A61P 29/00; C12N 15/86; C12N 2310/13; C12N 2310/315; C12N 2310/321; C12N 2750/14143

(57) ABSTRACT

The invention relates to the field of oligonucleotides that can inhibit a RNA-binding protein (RBP) such as Quaking (QKI) by acting as a binding sequence for said RBP (“decoys”). Such oligonucleotide may be used for the treatment of any disease or condition associated with an elevated expression level of QKI, such as inflammation or fibrosis.

### FIELD

The invention relates to the field of oligonucleotides that can inhibit a RNA-binding protein (RBP) such as Quaking (QKI) by acting as a binding sequence for said RBP (“decoys”). Such oligonucleotide may be used for the treatment of any disease or condition associated with an elevated expression level of QKI, such as inflammation or fibrosis.

### BACKGROUND OF THE INVENTION

Adaptations in cellular function in disease settings are associated with dynamic transcriptional and post-transcriptional changes in the levels of (pre-) mRNA species (de Bruin, R. G. et al., 2017, European Heart Journal; 38 (18): 1380-1388). The therapeutic targeting of factors that coordinate the levels of these transcripts in such situations could represent novel means of shifting cells and tissues from disease-advancing to regeneration-promoting. In this setting, RBPs have emerged as pivotal players, as they intimately govern all aspects of (patho) physiological RNA processing (FIG. 1).

Recent studies have led to the suggestion that the human genome encodes more than 700 RBPs.

The RBP QKI is a KH-domain containing protein and member of the highly conserved signal transduction and activator of RNA (STAR) family of RBPs (FIG. 2) (Darbelli, L. et al., 2016, Wiley Interdisciplinary Reviews in RNA; 7 (3): 399-412). The QKI locus resides on human chromosome 6 and transcription yields a pre-mRNA that yields 3 primary splice variants that contain the sequence information encoding the QKI-5, QKI-6 and QKI-7 protein isoforms. Importantly, these proteins are largely identical, aside from the fact that QKI-5 possesses 30 unique C-terminal amino acids, as opposed to 8 and 14 for QKI-6 and QKI-7, respectively. Of note, the unique C-terminus for QKI-5 possesses a nuclear localization signal (NLS) that is responsible for an almost exclusive detection in this portion of the cell (Wu, J. et al., 1999, Journal of Biological Chemistry; 274 (41): 29202-29210).

Augmentation of QKI protein expression has been observed in numerous cell types in response to injury, and is coupled with shifts to pro-fibrotic phenotypes (van der Veer, E. P. et al., 2013, Circulation Research, 113 (9): 1065-1075; de Bruin, R. G. et al., 2016, Nature Communications, 7:10846; de Bruin, R. G. et al., 2020, Epigenomics, 4 (2); Chothani, S. et al., 2019, Circulation; 140 (11): 937-951).

At present, there are no existing treatments geared towards the direct reduction of inhibition of an activity or of a function of a QKI protein as this protein represents a novel target in the inflammation and fibrosis setting. Two types of inflammation are most prevalent, namely acute and chronic inflammation. Acute inflammation in tissues is the direct result of trauma, pathogen invasion or accumulation of toxic compounds (Pahwa, R. et al., 2020, Chronic Inflammation, NBK493173) and can result in fibrosis. Fibrosis is defined as the excessive deposition of extracellular matrix (or connective tissue), and is commonly observed in the liver, heart, kidney, lungs, eyes and skin (Distler, J. H. W. et al., 2019, Nature Reviews Rheumatology, 15, 705-730). Chronic inflammation, resulting in excessive tissue fibrosis, is the direct result of slow, long-term inflammation that lasts months to years. 60% of people die as a result of the complications of chronic inflammation and fibrosis (ex: stroke, chronic obstructive pulmonary disease, idiopathic pulmonary fibrosis, heart disorders, cancer, obesity, diabetes and autoimmune diseases).

Most features of acute inflammation are also present in the chronic setting, including expansion of blood vessels and capillaries, neutrophil accumulation in damaged/infected tissue, which progresses from monocyte recruitment, infiltration and conversion to macrophages, local release of cytokines, subsequent attraction of dendritic cells and lymphocytes (and mast cells) that collectively drive tissue-resident cells to elaborate excessive connective tissue (Pahwa, R. et al., 2020, Chronic Inflammation, NBK493173).

To limit inflammation, current approaches include dietary options (such as low glycemic diets; fruits and vegetables; additional fiber; fish oils and micronutrient supplementation). Increased exercise is also recommended. Finally, several anti-inflammatory drugs are currently prescribed for patients with inflammatory disorders: 1) Metformin: mediates reductions in TNF-α, IL-1β, CRP and fibrinogen. Drawbacks of their use include physical weakness, abdominal pain (gas and diarrhea), myalgia and respiratory tract infections: 2) Statins: These drugs are particularly effective in reducing levels of circulating low-density lipoprotein levels. Drawbacks of their use include an increased risk of developing type II diabetes, liver and kidney damage, muscle weakness/damage and memory loss; 3) Non-steroidal anti-inflammatory drugs (NSAIDs): such naproxen, acetaminophen, ibuprofen and aspirin are inhibitors of cyclooxygenases that drive inflammatory responses. Drawbacks of their use include allergic reactions, gastrointestinal problems, kidney damage, increased risk of heart and stroke disease and skin reactions; 4) Corticosteroids: these drugs, such as prednisone and cortisone, reduce the activity of the immune system. Drawbacks of their use include increased risk of infections, fatigue, loss of appetite or weight gain, myalgia and thinning skin; 5) Immunosuppressives: drugs such as tacrolimus, sirolimus and mycophenolate mofetil are anti-lymphocyte agents by inhibiting their proliferation/expansion. Drawbacks of their use include serious risk of infection, liver and kidney damage; 6) Herbal supplements: such as ginger, turmeric and cannabis, through various mechanisms. Drawbacks of their use include allergic reactions, headaches nausea and diarrhea. Importantly, herbal supplements are not FDA approved (Pahwa, R. et al., 2020, Chronic Inflammation, NBK493173).

Several anti-fibrotic drugs are currently also employed, in particular in patients with idiopathic pulmonary fibrosis, namely: 1) Nintedanib: This drug is a vascular endothelial growth factor receptor (VEGFR) inhibitor that interferes with fibroblast proliferation, differentiation and extracellular matrix production, and has also been studied in the reduction of lung cancer. Drawbacks of its' use include abdominal pain, vomiting and diarrhea; 2) Sunitinib: This small molecule drug is a receptor-tyrosine kinase (RTKs) inhibitor that targets multiple RTKs involved in tumour growth and angiogenesis. Adverse effects associated with this drug include fatigue, nausea, diarrhea and hypertension; 3) Pirfenidone: This small molecule is a cytochrome P450 inhibitor whereby it inhibits growth factor production and procollagen I and II synthesis. Drawbacks of use of this agent include gastrointestinal complications, photosensitivity, liver damage, dizziness and weight loss.

Therefore, there is still a need for treatment for diseases or conditions associated with QKI.

### SUMMARY OF THE INVENTION

In an aspect, there is provided an oligonucleotide comprising a core QKI binding site UACUAAY and optionally a half QKI binding site YAAY, wherein Y is C or U and which is able to bind a QKI protein and as a result is able to inhibit an activity of said QKI protein.

In an embodiment, this oligonucleotide comprises two core QKI binding sites UACUAAC and no half QKI binding site and the length of the oligonucleotide is from 14 to 40 nucleotides, preferably 13 to 28 nucleotides.

In an embodiment, this oligonucleotide comprises one core QKI binding site UACUAAC and one half QKI binding site YAAY, and the length of the oligonucleotide is from 12 to 39 nucleotides, preferably 13 to 28 nucleotides.

In an embodiment, this oligonucleotide comprises only one QKI core binding site UACUAAY and no half QKI binding site and the length of the oligonucleotide is from 7 to 22 nucleotides, preferably 9 to 18 or 11 to 18 nucleotides.

In an embodiment, this oligonucleotide is such that the core and the half QKI binding sites are separated by 1-20 nucleotides, preferably 5-15 nucleotides

In an embodiment, this oligonucleotide is such that the half QKI binding site is present upstream/5′ side of the core QKI binding site.

In an embodiment, this oligonucleotide is such that the half QKI binding site is present downstream/3′ side of the core QKI binding site.

In an embodiment, the oligonucleotide comprises, consists of or consists essentially of (ACUAAY) n wherein Y is C or U and n is an integer ranged from 1 to 6 (n=2-6 correspond to SEQ ID NO: 100-104 respectively), preferably wherein Y is C. In an embodiment, the length of such oligonucleotide is ranged from 6 to 50 nucleotides.

In an embodiment, the oligonucleotide comprises, consists of or consists essentially of (UACUAAY) n wherein Y is C or U and n is an integer ranged from 1 to 6 (n=2-6 correspond to SEQ ID NO: 106-110), preferably wherein Y is C. In an embodiment, the length of such oligonucleotide is ranged from 6 to 50 nucleotides.

In an embodiment, this oligonucleotide is conjugated to a peptide, vitamin, aptamer, carbohydrate or mixtures of carbohydrates, protein, small molecule, antibody, polymer, drug, lithocholic acid, eicosapentanoic acid or a cholesterol moiety. In an embodiment, the conjugation is at its 3′ end.

In an embodiment, this oligonucleotide is such that a GalNac moiety has been conjugated to it's 5′ or 3′ end.

In an embodiment, this oligonucleotide is such that a small molecule, aptamer or antibody has been conjugated to it, either at the 5′ or 3′ end.

In an embodiment, the oligonucleotide is a single stranded oligonucleotide.

In an embodiment, this oligonucleotide is a modified RNA oligonucleotide comprising a nucleotide analogue and/or a modified internucleotide linkage.

Preferably, the nucleotide analogue comprises a modified base and/or a modified sugar and/or wherein a modified internucleotide linkage and more preferably wherein the internucleotide linkage is a phosphorothioate internucleotide linkage.

In a preferred embodiment, the backbone of the central part of the oligonucleotide has not been modified and preferably the internucleotide linkages at the 2 to 4 most 5′ end and/or 2 to 4 most 3′ end of the oligonucleotide have been modified, preferably as phosphorothioate internucleotide linkage.

In a preferred embodiment, the oligonucleotide is as follows: GCUUUACUAACACAGUACUAACAUCG (SEQ ID NO:11), wherein the underlined nucleotides have a phosphorothioate linkage and all nucleotides have a 2-O′ methyl base.

In an embodiment, the oligonucleotide comprises, consists of or essentially consists of SEQ ID NO: 55, 57, 59, 61, 63, 65, 66, 68, 69, 70, 71, 72, 73, 74, 78, 79, 81, 82, 90, 91, 92, 93, 94, 95, 96, 97, 98, 99, 100, 101, 102, 103, 104, 105, 106, 107, 108, 109,110, 111, 112, 113, 114, 115, 116, 117, 118.

In another aspect, there is provided a viral vector comprising a nucleic acid sequence encoding the oligonucleotide as defined herein.

In another aspect, there is provided a composition comprising the oligonucleotide as defined herein or a viral vector as defined herein.

In another aspect, there is provided an oligonucleotide or a viral vector or a composition, which are for use as a medicament. Preferably, the medicament is for treating a disease or condition associated with an elevated expression level of QKI. More preferably, wherein the disease or condition is an inflammatory disease or condition. Even more preferably, the inflammatory disease or condition is fibrosis. In an embodiment, this oligonucleotide or viral vector or composition for use is able to induce a therapeutic activity, effect, result in such disease or condition.

### BRIEF DESCRIPTION OF THE DRAWINGS

FIG. 1: RNA-binding proteins impact the fate of target transcripts. RNA-binding proteins (RBPs) are regulators of RNA fate in that they interact with target RNAs. This can occur with both pre-mRNAs and mature mRNAs, where they can influence splicing decisions (A-C), polyadenylation of the 3′ terminus (D), localization within the cell (E), transcript stability (F-G) and the levels of translation of such RNAs (H). Figure taken from de Bruin, R. G. et al., 2017, European Heart Journal, 38 (18): 1380-1388.

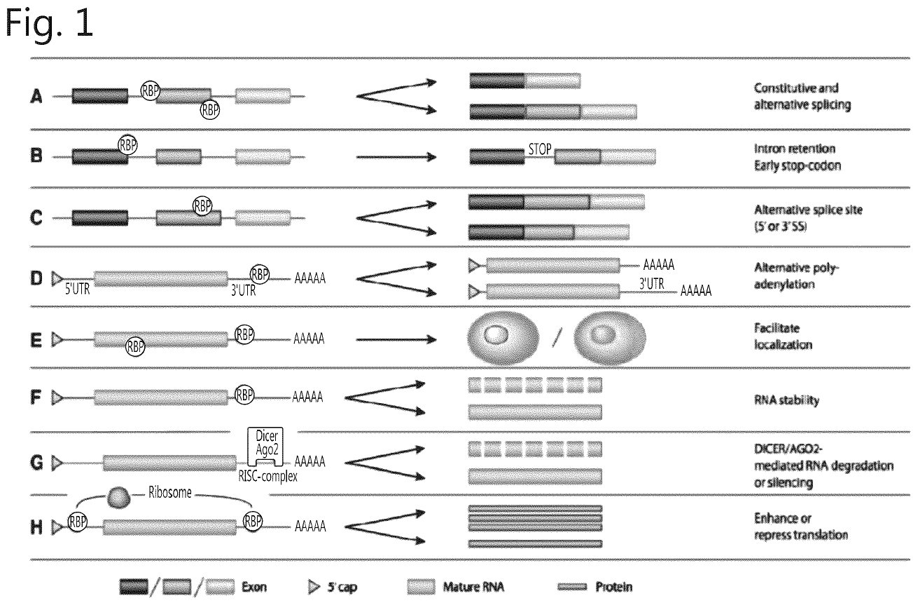

FIG. 2: Detailed description of the RBP Quaking (QKI). QKI possesses three main isoforms, generated by alternative splicing of the QKI pre-mRNA, resulting in the formation of QKI-5, QKI-6 or QKI-7. All 3 isoforms possess a single KH-domain for RNA-binding, and are identical in protein sequence from the N-terminus to the C-terminal residue 311. From this point, QKI-5 possesses 30 unique amino acid residues that also contain a nuclear localization signal. Hence the almost exclusive compartmentalization of QKI-5 in the nucleus of cells (with immunohistochemical staining). In contrast, the cytoplasmic QKI-6 and QKI-7 isoforms possess 8 and 14 unique C-terminal amino acids. Via homo- and heterodimerization, QKI-QKI dimers bind to their consensus sequence, where a 1-20 nucleotide spacer region separates the core-site (UACUAAC) and half-site (UAAC). Importantly, heterodimerization can impact the subcellular localization of individual isoforms, whereby heterodimerization between QKI-5 and either of the QKI-6 or QKI-7 isoforms can also lead in certain cell-types to nuclear localization. Note: the QRE for QKI was initially experimentally determined by Galarneau and Richard (Galarneau, A. et al., 2005, Nature Structural & Molecular Biology, 12 (8): 691-698).

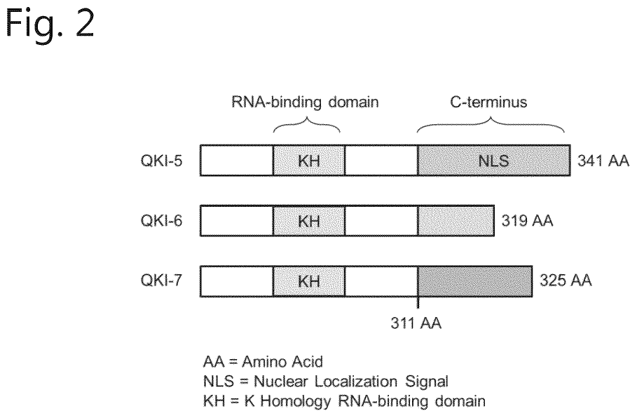

FIG. 3: QKI expression is induced in the kidney upon injury. Ischemic reperfusion injury to C57BL6 mice results in an increase in nuclear QKI-5 expression (bottom left panel; see solid arrows), QKI-6 (middle panel; see solid arrows) with clear augmentation in proximal tubules, and QKI-7 (bottom right panel, see solid arrows), where a clear shift from negative nuclei in healthy kidney (top right panel) is found now to be diffusely nuclear and cytoplasmic. Glomerular staining for QKI appears to be relatively unaffected by injury relative to healthy controls. Also notable is the massive influx of mononuclear cells in the kidney interstitium that are highly positive for QKI-5 expression (bottom left panel; dashed arrows) and are clearly evident in the bottom panels stained for QKI-6/7 (dashed arrows).

FIG. 4: QKI isoform levels with the kidney display cell-type specific profiles. Western blot analysis of QKI isoform expression in whole cell lysates harvested from numerous human and mouse kidney cell lines. Cell lines utilized for this study include proximal tubuli (PTECs), collecting duct (IMCD), interstitial fibroblasts (3T3, TK173), adult mesangial cells (AMC) and endothelium (venous compartment, HUVEC; glomerular compartment, GENC). Beta-actin serves as a loading control, and data are representative of n=3.

FIG. 5: TGF-β stimulates QKI mRNA expression. Stimulation of human interstitial fibroblasts with TGF-β results in increased QKI-5, QKI-6 and QKI-7 mRNA expression, albeit with clear differences in the timing of such increases. In particular, a striking increase in QKI-6 was observed at 24 h post-stimulation. Evidence of TGF-β responsiveness is clear with increased expression of smooth muscle α-actin (ASMA), indicating that the TK173 cells have become activated and are undergoing the conversion to the myofibroblast phenotype. Data are representative of n=3 biological replicates, where *p<0.05, **p<0.01 and ***p<0.001.

FIG. 6: Design of an RNA-based inhibitor of QKI activity with a single core and single half site separated by a spacer region. In efforts to diminish target engagement for QKI within cells, several RNA-based approaches can be envisioned. As opposed to employing oligonucleotides to degrade QKI mRNAs, we designed oligonucleotides that inhibit QKI activity. For this, we developed oligonucleotides of 29 and 25 nucleotides in length, namely QRE-D1 (SEQ ID NO:47) and QRE-D2 (SEQ ID NO:48), respectively. Importantly, the half-site was downstream in QRE-D1 (SEQ ID NO:47) and upstream in QRE-D2 (SEQ ID NO:48). Furthermore, the spacer regions between the core and half-site varied by 10 and 6 nucleotides, respectively. Mutated controls MUT-QRE-1 (SEQ ID NO:49) and MUT-QRE-2 (SEQ ID NO:50) possessed a guanine residue in the core site U residue (UACGAAC vs. UACUAAC). Cholesterol was added as a conjugate to the 3′ end of the ‘decoy’ to improve cellular uptake, while a DY647 conjugate was added to the 5′ end to improve visualization of decoy uptake. All residues possess a O-Me modification of the 2′-position of the sugar moiety to limit endonuclease-mediated degradation, while phosphorothioates were incorporated at the 2 most 5′-end nucleotides and 4 most 3′-end nucleotides. B) Uptake in HEK293 cells was comparable for both QRE-D1 (SEQ ID NO:47) and QRE-D2 (SEQ ID NO:48) as assessed by flow cytometry and dose-dependent increases in uptake were observed. C) Treatment of HITC6 vascular smooth muscle cells with the denoted oligonucleotides revealed changes in myocarding (myocd) splicing, with QKI decoy treatment yielding increased production of the VSMC-enriched transcript commonly observed upon reduction of QKI expression (as described in van der Veer, E. P. et al. 2013, Circulation Research, 113 (9): 1065-1075). SEQ ID NO: 47 is QRE-D1 with DY647 and cholesterol. SEQ ID NO: 48 is QRE-D2 with DY647 and cholesterol. SEQ ID NO: 115 is QRE-D1 with cholesterol and no DY647. SEQ ID NO: 116 is QRE-D2 with cholesterol and no DY647. SEQ ID NO: 117 is QRE-D1 with no cholesterol and no DY647. SEQ ID NO: 118 is QRE-D2 with no cholesterol and no DY647.

FIG. 7: Hyperoxia in a rat bronchopulmonary dysplasia model results in increased QKI expression. Left panel: QKI-5, QKI-6 and QKI-7 are readily expressed in healthy lung tissue (left), with minimal SM-α-actin (ASMA) staining evident in healthy lung tissue. Right panel: Exposure of rate pups to 90% O2 for 9 days (hyperoxia) results in markedly increased levels of QKI-5 and QKI-7, with but a moderate increase in QKI-6 expression observed in these conditions. The clear damage to the lung tissue by hyperoxia is evidenced by dilated bronchi and significant increases in ASMA staining (Br, bronchi; a, alveoli; Ar, arteries).

FIG. 8: Design of dual core sites decoy separated by a spacer region for inhibition of QKI activity. In efforts to diminish target engagement for QKI within cells, we designed oligonucleotides that inhibit QKI activity. These oligonucleotides were 27 nucleotides in length and possess a dual QRE core element. This element was spaced by 4 nucleotides from a second core sequence (as opposed to a half site) in efforts to generate multiple ‘optimal’ binding sites for QKI that also possess sufficient binding specificity. Guanine residues were introduced at 2 positions in the core site of the decoy, namely UACGAAC. Cholesterol was added as a conjugate to the 3′ end of the ‘decoy’ to improve cellular uptake, while a DY647 conjugate was added to the 5′ end to improve visualization of the decoy in vivo. All residues possess a O-Me modification of the 2′-position of the sugar moiety to limit endonuclease-mediated degradation, while phosphorothioates were incorporated at the 2 most 5′-end nucleotides and 4 most 3′-end nucleotides. The absence of phosphorothioates in the middle portion of the decoy is to allow for maximal chirality (flexibility) to allow for maximal binding capacity of QKI with the core sequence(s).

FIG. 9: QKI decoys are actively taken up lungs of neonatal rats. RNA-Cont-1 (SEQ ID NO:22) and RNA-QRE-1 (SEQ ID NO:24) oligonucleotides were administered at a concentration of 40 mg/kg on day subcutaneously on day 2 post-birth. Oligonucleotides display clear uptake in the bronchi, alveoli and arteries of lung tissue.

FIG. 10: Inhibition of QKI does not impact alveolar enlargement, but attenuates septal thickness in experimental BPD. Representative HE-stained lung sections (A-C) in rat pups kept in RA (A) or 100% O2 (B and C) until 10 days of age. Quantifications of alveolar crests and septal thickness was determined on paraffin sections in Wistar rats on day 10 in RA (open bar) or hyperoxia (shaded bars). Pups were injected intraperitoneally on day 2 with 40 mg/kg of RNA-Cont-1 (SEQ ID NO:22) and RNA-QRE-1 (SEQ ID NO:24) oligonucleotides dissolved in 100 μl 0.9% NaCl. Values are expressed as mean±SEM. ***p<0.001 versus RA controls. †††p<0.001 versus age-matched O2-exposed controls. Two independent experiments were performed. a=alveolus.

FIG. 11: Inhibition of QKI attenuates neutrophilic granulocytic influx in experimental BPD. Representative lung sections stained for the neutrophilic granulocyte marker myeloperoxidase (MPO; panels A-C) in rat pups kept in RA (A) or 100% 02 (B and C) until 10 days of age. Quantifications of the pulmonary influx of neutrophils was determined on paraffin sections in Wistar rats on day 10 in RA (open bar) or hyperoxia (shaded bars). Pups were injected intraperitoneally on day 2 with 40 mg/kg of RNA-Cont-1 (SEQ ID NO:22) and RNA-QRE-1 (SEQ ID NO:24) oligonucleotides dissolved in 100 μl 0.9% NaCl. Values are expressed as mean±SEM. *p<0.05 and ***p<0.001 versus RA controls. †††p<0.001 versus age-matched O2-exposed controls. Two independent experiments were performed. a=alveolus.

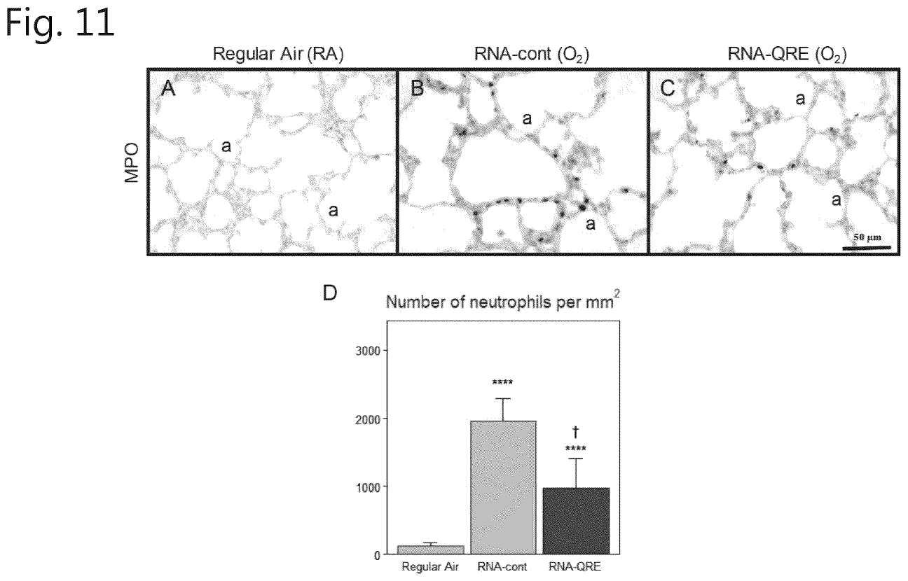

FIG. 12: Inhibition of QKI attenuates the influx of macrophages in experimental BPD. Representative lung sections stained for the macrophage marker ED1 (A-C) in rat pups kept in RA (A) or 100% O2 (B and C) until 10 days of age. Quantifications of the pulmonary influx of macrophages was determined on paraffin sections in Wistar rats on day 10 in RA (open bar) or hyperoxia (shaded bars). Pups were injected intraperitoneally on day 2 with 40 mg/kg of RNA-Cont-1 (SEQ ID NO:22) and RNA-QRE-1 (SEQ ID NO:24) oligonucleotides dissolved in 100 μl 0.9% NaCl. Values are expressed as mean±SEM. ***p<0.001 versus RA controls. †††p<0.001 versus age-matched 02-exposed controls. Two independent experiments were performed. a=alveolus.

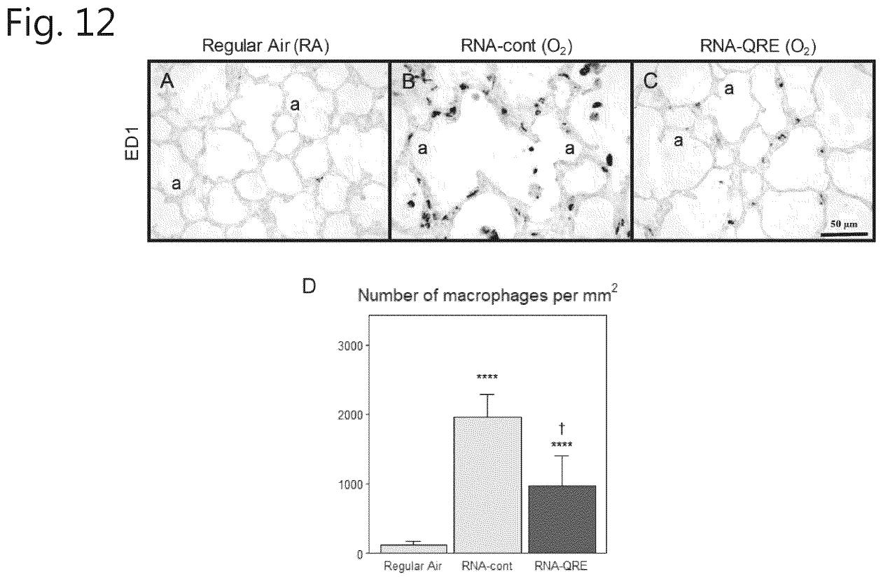

FIG. 13: Inhibition of QKI leads to diminished Col3A expression in experimental BPD. Representative lung sections stained for the fibrotic marker collagen 3 (col3; panels A-C) in rat pups kept in RA (A) or 100% 02 (B and C) until 10 days of age. Quantifications of col3 expression was determined on paraffin sections in Wistar rats on day 10 in RA (open bar) or hyperoxia (shaded bars). Pups were injected intraperitoneally on day 2 with 40 mg/kg of RNA-Cont-1 (SEQ ID NO:22) and RNA-QRE-1 (SEQ ID NO:24) oligonucleotides dissolved in 100 μl 0.9% NaCl. Values are expressed as mean±SEM. ***p<0.001 versus RA controls. ††p<0.01 versus age-matched O2-exposed controls. Two independent experiments were performed. a=alveolus.

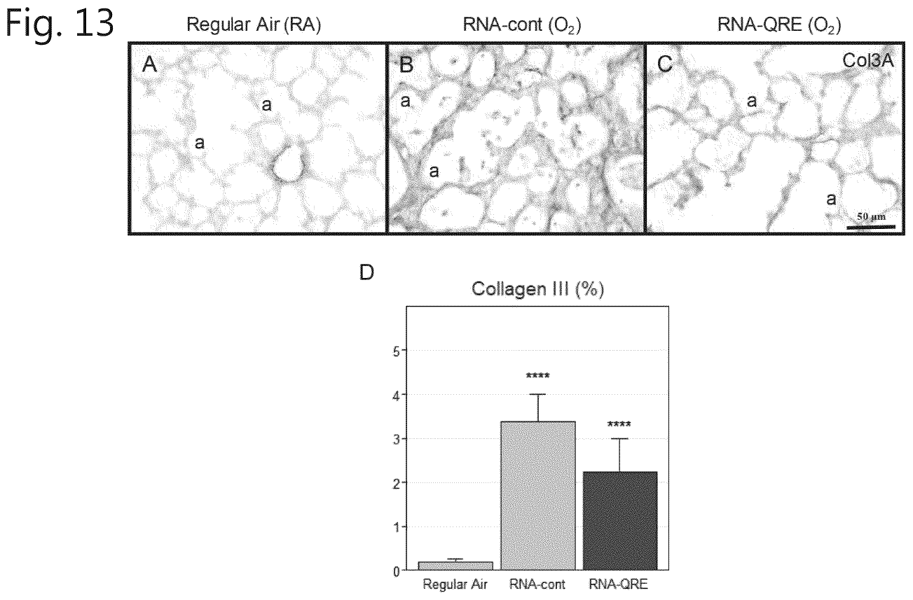

FIG. 14: QKI inhibition minimally impacts vascular remodeling and right ventricular hypertrophy in experimental BPD. Representative lung sections stained for α-smooth muscle actin (ASMA; panels A-C) in rat pups kept in RA (A) or 100% 02 (B and C) until 10 days of age. Quantifications of medial wall thickness of the ASMA positive layer of small arterioles as a marker for arterial pulmonary hypertension was determined on paraffin lung sections and of the RV/LV ratio as a marker for right ventricular hypertrophy was determined on paraffin heart sections in Wistar rats on day 10 in RA (open bar) or hyperoxia (shaded bars). Pups were injected intraperitoneally on day 2 with 40 mg/kg of RNA-Cont-1 (SEQ ID NO:22) and RNA-QRE-1 (SEQ ID NO:24) oligonucleotides dissolved in 100 μl 0.9% NaCl. Values are expressed as mean±SEM. ***p<0.001 versus RA controls. ††p<0.01 versus age-matched O2-exposed controls. Two independent experiments were performed. art=arteriole.

FIG. 15: QKI-5 protein expression is increased in human kidney pathologic conditions. QKI-5 protein is abundantly detected is nuclei of both healthy and diseased kidney material. In disease settings such as metabolic syndrome, focal segmental glomerularsclerosis and (acute) rejection (panels 2-4), QKI-5 protein expression is slightly augmented in nuclei relative to healthy kidney material, in particular in metabolic syndrome and acute rejection kidneys (see solid arrows).

FIG. 16: QKI-6 protein expression is increased in human kidney pathologic conditions. QKI-6 protein expression is increased in human kidney pathologic conditions. QKI-6 is expressed in the distal tubules (solid arrows) of healthy kidneys while being poorly expressed in the abundant proximal tubules (dotted arrows) of the kidney cortex (left panel). Glomerular staining (gl) for QKI-6 is evident, albeit moderate (left panels, see gl). In disease settings such as metabolic syndrome, focal segmental glomerularsclerosis and (acute) rejection (panels 2-4), QKI-6 protein is clearly abundantly expressed in distal and proximal tubules, and increased in expression in glomerular cells (gl).

FIG. 17: QKI-7 protein expression is slightly increased in human kidney pathologic conditions. While QKI-7 protein is clearly expressed in proximal and distal tubules of healthy kidney, glomerular staining is relatively mild. QKI-7 protein expression is clearly augmented in glomerular cells of diseased human kidney, along with slightly increased QKI-7 expression in nuclei of proximal and distal tubular epithelial cells, as evidenced by dark brown nuclear staining. Furthermore, apical accumulation of QKI-7 is evident in the diseased tissue sections (see arrows).

FIG. 18: QKI isoforms are differentially expressed in the kidney following unilateral ureter obstruction (UUO). UUO in C57BL6 mice results in an increase in nuclear QKI-5 expression (left panel), QKI-6 (middle panel) with augmentation in distinct cortical regions and attenuation in others, a pattern that was mimicked for QKI-7 (right panel). Glomerular staining for QKI appears to be relatively unaffected by injury relative to healthy controls.

FIG. 19: Body weight is not affected by treatment with QKI-inhibiting dcRNA. A. Experimental setup of UUO injury model in C57Bl6 mice to assess potential kidney protective effects of a QKI-inhibiting dcRNA. UUO was performed by introducing a double ligation of the left ureter. Mice were sacrificed either 5 or 10 days post-injury. B. Administration of SEQ ID NO:54 or SEQ ID NO:55 did not impact body weight between day-1 and day 0 nor following administration of a second dose of SEQ ID NO: 54 or SEQ ID NO: 55 2 days post-injury (in both the day 5 and day 10 mouse groups). Post-UUO a clear drop in body weight was observed in both groups of mice which was partially recovered in the day 5 group of mice and fully recovered in the day 10 group of mice. N=12 mice per treatment arm.

FIG. 20: Kidney weight is not affected by treatment with QKI-inhibiting dcRNA. Administration of SEQ ID NO:54 or SEQ ID NO:55 did not impact contralateral (CLK) nor injured (UUO) kidney weight following harvesting on day 5 or 10 post-injury. N=12 mice per treatment arm.

FIG. 21: dcRNAs display excellent distribution to the kidney. QKI decoys are actively taken up in the kidneys of C57Bl6 mice. SEQ ID NO:54 and SEQ ID NO:55 were administered intravenously at a concentration of 40 mg/kg on day 1 prior to injury and 2 days post-injury. Oligonucleotides were detected using an antibody detecting phosphorothioate-modified residues. N=12 mice per treatment arm.

FIG. 22: Serum urea levels are not affected by treatment with QKI-inhibiting dcRNA. Serum urea levels with increased in both the day 5 (left panel) and day 10 (right panel) mouse groups post-UUO. However, no significant differences in serum urea levels were observed based on treatment with either SEQ ID NO: 54 or SEQ ID NO: 55 in the day 5 or day 10 mouse groups. N=12 mice per treatment arm.

FIG. 23: Treatment with Quaking-inhibiting dcRNA reduces macrophage infiltration in UUO-injured mice. Representative kidney sections stained for the macrophage marker F4/80 in mice either 5 days or 10 days post UUO (left and right panels, respectively). Quantification of the kidney macrophage accumulation was determined on paraffin sections harvested from UUO-injured C57Bl6 mice treated with either SEQ ID NO: 54 (light grey bars) or SEQ ID NO: 55 (dark grey bars). Data are indicative of n=12 mice per treatment arm, where **p<0.01.

FIG. 24: Treatment with Quaking-inhibiting dcRNA reduces collagen accumulation in UUO-injured mice. Inhibition of QKI leads to diminished collagen accumulation as evidenced by decreased picrosirius red staining in the kidney interstitium at 5 and 10 days post-UUO (left and right panels, respectively). Quantification of the collagen accumulation was determined on paraffin sections harvested from UUO-injured C57Bl6 mice treated with either SEQ ID NO: 54 (light grey bars) or SEQ ID NO: 55 (dark grey bars). Data are indicative of n=12 mice per treatment arm, where **p<0.01 and *p<0.05.

FIG. 25: Distinct QKI protein isoforms bind with varying affinity to dcRNAs. Western blot of dcRNA binding to A) QKI-5, B) QKI-6 and C) QKI-7 following pull-down with antibodies specific for the distinct QKI isoforms with lysates derived from nuclear (left lanes in all gels) and cytoplasmic fractions (right lanes in all gels). A scrambled oligonucleotide was used as ‘control’.

FIG. 26: Distinct QKI protein isoforms bind with varying affinity to dcRNAs. A) Western blot of HEK293-derived cellular lysates wherein biotin-labelled dcRNAs were bound by QKI-5 following streptavidin pull-down. B) Distinct compositions and spacing of QKI consensus motifs impacts relative binding affinity to QKI-5 for the respective dcRNAs.

FIG. 27: QKI-inhibiting dcRNA treatment alters splicing of QKI-target pre-mRNAs. A) RT-PCR for ADD3 (top panel) and ACTB (bottom panel) revealing splice modulation mediated by treatment with SEQ ID NO: 64 (mutated dcRNA) and SEQ ID NO: 65. B) Quantification of ADD3 exclusion (excl.) vs. inclusion (incl.) ratio in SEQ ID NO: 64 and SEQ ID NO: 65-treated HEK293 cells revealing dose-dependent effect on splice modulation using QKI-inhibiting dcRNA.

### DESCRIPTION

### Oligonucleotide

The inventors surprisingly discovered that several types of oligonucleotides could be used for binding to QKI (i.e. they comprise a QKI binding site such as a QKI core and/or a half binding site) and as a result could be used for inhibiting a QKI activity. Such oligonucleotides are described below in more detail. Such oligonucleotides will be referred to herein as oligonucleotides according to the invention.

Throughout the application, in an embodiment, an oligonucleotide of the invention may be able to bind a QKI protein and as a result may be able to inhibit an activity of said QKI protein.

Throughout the application, in an embodiment, an oligonucleotide of the invention is able to bind a QKI protein and as a result is able to inhibit an activity of said QKI protein.

QKI is the name of a RNA binding protein and also the name of the encoding gene. According to the context, it is clear to the skilled person whether the abbreviation QKI refers to the protein or to the gene. Transcription of the QKI gene leads to three primary splice variants that contain the sequence information encoding the QKI-5, QKI-6 and QKI-7 protein isoforms. Therefore, the QKI protein is synonymous with the QKI-5, QKI-6 and/or QKI-7 proteins. Importantly, these proteins are largely identical, aside from the fact that QKI-5 possesses 30 unique C-terminal amino acids, as opposed to 8 and 14 for QKI-6 and QKI-7, respectively. Of note, the unique C-terminus for QKI-5 possesses a nuclear localization signal (NLS) that is responsible for an almost exclusive detection in this portion of the cell (Wu, J. et al., 1999, Journal of Biological Chemistry; 274 (41): 29202-29210).

Therefore, a core QKI binding site or a half QKI binding site is a site that can bind the QKI protein, i.e. any of QKI-5, QKI-6 and/or QKI-7. As a result, an inhibition of an activity of the QKI protein means an inhibition of an activity of any of QKI-5, QKI-6 and/or QKI-7.

In a first aspect, there is provided an oligonucleotide comprising a core QKI binding site UACUAAY and optionally a half QKI binding site YAAY, wherein Y is C or U. It is clear to the skilled person that this optional half QKI binding site when present in said oligonucleotide is present as a separate or distinct or additional motif present next to the core QKI binding site. In other words, the core QKI binding site cannot be considered to encompass or comprise a half QKI binding site in the context of the application.

Throughout the application, the expression “bind” or “binding site” is used in the context of the oligonucleotide which is able to bind QKI (i.e. QKI-5, QKI-6 and/or QKI-7, preferably QKI-5) or which comprises a binding site for QKI (i.e. QKI-5, QKI-6 and/or QKI-7, preferably QKI-5). Each oligonucleotide as defined in the invention exhibits at least some detectable level of QKI binding and/or some detectable level of QKI-inhibiting activity (i.e. QKI-5, QKI-6 and/or QKI-7, preferably QKI-5). An oligonucleotide will be said to bind QKI or to comprise a binding site for QKI when it will be able to bind at least 20%, 30%, 40%, 50%, 60%, 70%, 80%, 90%, 95%, 99% of the available QKI. This binding may be assessed using EMSA (Electrophoretic Mobility Shift Assay) using the oligonucleotide of the invention comprising said putative binding site as a probe and incubating it with QKI. The inclusion of a guanosine nucleotide in the middle position of a core site sequence in such oligonucleotides (middle position where for example UACUAAC is mutated to UACGAAC will serve as a control or comparator for QKI-binding/inhibiting oligonucleotides. These controls will be equivalent in length to QKI-inhibiting oligonucleotides. In an embodiment, this binding leads to an inhibition of a QKI activity. The inhibition of a QKI activity may be assessed using techniques known to the skilled person. The inhibition may be of at least 20%, 30%, 40%, 50%, 60%, 70%, 80%, 90%, 95%. 99% of the initial activity. Given that QKI (i.e. QKI-5, QKI-6 and/or QKI-7, preferably QKI-5) is a well-defined regulator of alternative splicing (Darbelli, L. et al., 2016, Wiley Interdisciplinary Reviews in RNA; 7 (3): 399-412; de Bruin, R. G. et al., 2016, Nature Communications, 7:10846), the degree of QKI inhibition will be assessed by determining the exon inclusion/exclusion ratios of well-defined QKI-regulated splicing events (i.e. QKI-5, QKI-6 and/or QKI-7, preferably QKI-5), such as MYOCD (myocardin), ADD3, ERBB2IP, LAIR1 and/or UTRN amongst other potential possibilities. This may be done using techniques known to the skilled person. Such techniques include PCR. When a modulation of splicing of one of the above-identified pre-mRNAs had been identified, a modulation of a QKI activity (i.e. QKI-5, QKI-6 and/or QKI-7, preferably QKI-5) will be considered to have been assessed. This modulation of splicing is compared to the splicing activity of control samples/cells.

In this context, if QKI (i.e. QKI-5, QKI-6 and/or QKI-7, preferably QKI-5) is known to induce/increase the formation of a given splicing product of one of the above-identified pre-mRNAs, the assessment of a lower quantity of this splicing product will be considered as an inhibition of a QKI (i.e. QKI-5, QKI-6 and/or QKI-7, preferably QKI-5) activity. A lower quantity may mean at least 5% lower, 10%, 15%, 20%, 30%, 40%, 50%, 60%, 70%, 80%, 90% or 95%. The assessment may be done using PCR. This modulation of splicing is compared to the splicing activity of control samples/cells.

In this context, if QKI (i.e. QKI-5, QKI-6 and/or QKI-7, preferably QKI-5) is known to decrease/inhibit the formation of a given splicing product of one of the above-identified pre-mRNAs, the assessment of a higher quantity of this splicing product will be considered as an inhibition of a QKI (i.e. QKI-5, QKI-6 and/or QKI-7, preferably QKI-5) activity. A higher quantity may mean at least 5% higher, 10%, 15%, 20%, 30%, 40%, 50%, 60%, 70%, 80%, 90% or 95%. The assessment may be done using PCR. This modulation of splicing is compared to the splicing activity of control samples/cells.

In an embodiment, QKI decreases the inclusion of exon 2a of myocardin. Therefore the inhibition of a QKI activity may be the increase of the inclusion of exon 2a of myocardin. A higher quantity of said exon may mean at least 5% higher, 10%, 15%, 20%, 30%, 40%, 50%, 60%, 70%, 80%, 90% or 95%. The assessment may be done using PCR. This modulation of splicing is compared to the splicing activity of control samples/cells.

In an embodiment, QKI decreases the inclusion of exon 14 of ADD3. Therefore the inhibition of a QKI activity may be the increase of the inclusion of exon 14 of ADD3. A higher quantity of said exon may mean at least 5% higher, 10%, 15%, 20%, 30%, 40%, 50%, 60%, 70%, 80%, 90% or 95%. The assessment may be done using PCR. This modulation of splicing is compared to the splicing activity of control samples/cells.

In another embodiment of the invention, the base Y present in the core QKI binding site UACUAAY and/or in the half QKI binding site YAAY of the oligonucleotide of the invention, may be I (i.e, inosine) or a wobble base.

As known to the skilled person an oligonucleotide is a polymer of nucleotides or a polymer of nucleotides analogues. In other words, an oligonucleotide comprises or consists of repeating monomers. An oligonucleotide may comprise up to 50 nucleotides. Said oligonucleotide may have 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. In some embodiments, the oligonucleotide of the invention has a length of 7, 8, 9, 10, 11, 12, 13, 14, 15, 16, 17, 18, 19, 20, 21, or 22 nucleotides, and may be identified as an oligonucleotide having from 7 to 22 nucleotides. In some other embodiments, the oligonucleotide has a length from 14 to 40 nucleotides or 13 to 28 nucleotides or 12 to 39 nucleotides.

In a first embodiment of this aspect, the oligonucleotide comprises a core QKI binding site UACUAAY and a half QKI binding site YAAY, wherein Y is C or U.

Accordingly, the oligonucleotide of this first embodiment may comprise:

UACUAAY
and
YAAY,
UACUAAC
and
CAAC,
UACUAAU
and
UAAU,
UACUAAC
and
CAAU,
UACUAAC
and
UAAU,
UACUAAU
and
CAAU,
UACUAAU
and
UAAC,
UACUAAU
and
CAAC
or
UACUAAC
and
UAAC.

Accordingly the length of the oligonucleotide of this first embodiment is from 11 to 50 nucleotides: 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. The length may be from 12 to 40 or from 14 to 30 or from 17 to 25 nucleotides.

A preferred oligonucleotide comprises one core QKI binding site UACUAAY and one half QKI binding site YAAY, wherein Y is C or U and wherein the length of the oligonucleotide is from 12 to 39 nucleotides, preferably 13 to 28 nucleotides.

In a second embodiment of this aspect, the oligonucleotide comprises a core QKI binding site UACUAAY, wherein Y is C or U. In this second embodiment, the oligonucleotide does not comprise a half QKI binding site YAAY, wherein Y is C or U.

Accordingly, the oligonucleotide of this second embodiment may comprise:
UACUAAY,
UACUAAC or
UACUAAU.

Accordingly, the length of the oligonucleotide of this second embodiment is from 7 to 50 nucleotides: 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides. The length may be from 7 to 40 or from 10 to 30 or from 17 to 25 nucleotides.

A preferred oligonucleotide comprises only one QKI core binding site UACUAAY (wherein Y is C or U) and no half QKI binding site YAAY and preferably the length of the oligonucleotide is from 7 to 22 nucleotides, preferably 9 to 22, or 9 to 18 nucleotides. In an embodiment this oligonucleotide has a length of 7, 8, 9, 10, 11 or 12 nucleotides. In a preferred embodiment, the oligonucleotide has a length of 9 nucleotides.

In a third embodiment of this aspect, the oligonucleotide comprises two core QKI binding sites UACUAAY, wherein Y is C or U. In this third embodiment, the oligonucleotide does not comprise a half QKI binding site YAAY, wherein Y is C or U.

Accordingly, the oligonucleotide of this third embodiment may comprise:

| | (in other words, it comprises (UACUAAY)₂), |
|---|---|
| (SEQ ID NO: 52) | |
| | UACUAAY and UACUAAY, |
| | (in other words, it comprises (UACUAAC)₂), |
| (SEQ ID NO: 51) | |
| | UACUAAC and UACUAAC, |
| | (in other words, it comprises (UACUAAU)₂), |
| (SEQ ID NO: 53) | |
| | UACUAAU and UACUAAU, |
| | or |
| | UACUAAU and UACUAAC. |

Each motif having UACUAAC, UACUAAY or UACUAAU is not per se contiguous with the other motif UACUAAC, UACUAAY or UACUAAU. There could be additional nucleotides (1, 2, 3, 4 or more) between the two motifs.

In a preferred embodiment, the oligonucleotide comprises: UACUAAC and UACUAAC, (in other words, it comprises (UACUAAC) 2 (SEQ ID NO:51),

Accordingly, the length of the oligonucleotide of this third embodiment is from 14 to 50 nucleotides: 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides. The length may be from 14 to 40 or from 18 to 35 or from 17 to 30 or 13 to 28 nucleotides.

A preferred oligonucleotide comprising two core QKI binding sites UACUAAY wherein Y is C or U and no half QKI binding site YAAY wherein Y is C or U and preferably the length of the oligonucleotide is from 14 to 40 nucleotides, preferably 13 to 28 nucleotides. A preferred length of such oligonucleotide is 27 nucleotides.

In an embodiment, the oligonucleotide is such that the core and the half QKI binding sites are separated by 1-20 (i.e. 1 or 2 or 3 or 4 or 5 or 6 or 7 or 8 or 9 or 10 or 11 or 12 or 13 or 14 or 15 or 16 or 17 or 18 or 19 or 20) nucleotides, preferably 5-15 (i.e. 5 or 6 or 7 or 8 or 9 or 10 or 11 or 12 or 13 or 14 or 15) nucleotides. In an embodiment, the core and the half QKI binding sites are separated by 4 or 5 or 6 nucleotides, preferably by 4 nucleotides.

In an embodiment, the oligonucleotide is such that two core QKI binding sites are separated by 1-20 (i.e. 1 or 2 or 3 or 4 or 5 or 6 or 7 or 8 or 9 or 10 or 11 or 12 or 13 or 14 or 15 or 16 or 17 or 18 or 19 or 20) nucleotides, preferably 5-15 (i.e. 5 or 6 or 7 or 8 or 9 or 10 or 11 or 12 or 13 or 14 or 15) nucleotides. In an embodiment, the two core QKI binding sites are separated by 4 or 5 or 6 or 7 nucleotides, preferably by 4 nucleotides.

In an embodiment, the oligonucleotide comprises two core QKI binding sites that are separated by 3 nucleotides and it does not comprise a half QKI binding site. The length of such oligonucleotide may be from 17 to 40 nucleotides or any of 17, 18, 19, 20, 21, 22, 23, 24, 25, 26, 27, 28, 29, 30, 31, 32, 33, 34, 35, 36, 37, 38, 39, 40 nucleotides. A preferred oligonucleotide comprises or consists of or essentially consists of the base sequence GCCGUAACCACGUCUACUAACGCCG (SEQ ID NO:59).

In an embodiment, the oligonucleotide comprises two core QKI binding sites that are separated by 4 nucleotides and it does not comprise a half QKI binding site. The length of such oligonucleotide may be from 18 to 40 nucleotides or any of 18, 19, 20, 21, 22, 23, 24, 25, 26, 27, 28, 29, 30, 31, 32, 33, 34, 35, 36, 37, 38, 39, 40 nucleotides. A preferred oligonucleotide comprises or consists of or essentially consists of the base sequence GCUUUACUAACACAGUACUAACAUCG (SEQ ID NO:55).

In an embodiment, the oligonucleotide comprises two core QKI binding sites that are separated by 7 nucleotides and it does not comprise a half QKI binding site. The length of such oligonucleotide may be from 21 to 40 nucleotides or any of 21, 22, 23, 24, 25, 26, 27, 28, 29, 30, 31, 32, 33, 34, 35, 36, 37, 38, 39, 40 nucleotides. A preferred oligonucleotide comprises or consists of or essentially consists of the base sequence GCUUUACUAACACUCACCUACUAACAUCG (SEQ ID NO:57).

In an embodiment, the oligonucleotide comprises two core QKI binding sites that are separated by 5 nucleotides and it does not comprise a half QKI binding site. The length of such oligonucleotide may be from 19 to 40 nucleotides or any of 19, 20, 21, 22, 23, 24, 25, 26, 27, 28, 29, 30, 31, 32, 33, 34, 35, 36, 37, 38, 39, 40 nucleotides. A preferred oligonucleotide comprises or consists of or essentially consists of the base sequence GCUUUACUAACACAGAUACUAACAUCG (SEQ ID NO:61).

Further preferred oligonucleotides derived from SEQ ID NO: 59, 55, 57 or 61 are later disclosed herein.

In a further embodiment, there is provided an oligonucleotide comprising a core QKI binding site ACUAAY wherein Y is C or U, preferably the core QKI binding site is ACUAAC. The length of such oligonucleotide may be from 17 to 40 nucleotides or any of 17, 18, 19, 20, 21, 22, 23, 24, 25, 26, 27, 28, 29, 30, 31, 32, 33, 34, 35, 36, 37, 38, 39, 40 nucleotides.

In a preferred embodiment, the oligonucleotide comprises, consists of or consists essentially of (ACUAAY) n wherein Y is C or U and n is an integer ranged from 1 to 6 (n=2 correspond to SEQ ID NO: 100-104 respectively) preferably the core QKI binding site is ACUAAC. The length of such oligonucleotide may be from 6 to 40 nucleotides or any of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides.

In a preferred embodiment, the oligonucleotide comprises, consists of or consists essentially of (ACUAAY) 1 wherein Y is C or U preferably the core QKI binding site is ACUAAC.

In a preferred embodiment, the oligonucleotide comprises, consists of or consists essentially of (ACUAAY) 2 wherein Y is C or U (SEQ ID NO: 100) preferably the core QKI binding site is ACUAAC. A preferred oligonucleotide consists of or consists essentially of (ACUAAC) 2 (SEQ ID NO: 70).

In a preferred embodiment, the oligonucleotide comprises, consists of or consists essentially of (ACUAAY) 3 wherein Y is C or U (SEQ ID NO:101) preferably the core QKI binding site is ACUAAC. A preferred oligonucleotide consists of or consists essentially of (ACUAAC) 3 (SEQ ID NO: 71).

In a preferred embodiment, the oligonucleotide comprises, consists of or consists essentially of (ACUAAY) 4 wherein Y is C or U (SEQ ID NO:102) preferably the core QKI binding site is ACUAAC. A preferred oligonucleotide consists of or consists essentially of (ACUAAC) 4 (SEQ ID NO: 72). SEQ ID NO: 78 corresponds to SEQ ID NO:72 further comprising a C6 biotin.

In a preferred embodiment, the oligonucleotide comprises, consists of or consists essentially of (ACUAAY) 5 wherein Y is C or U (SEQ ID NO:103) preferably the core QKI binding site is ACUAAC.

In a preferred embodiment, the oligonucleotide comprises, consists of or consists essentially of (ACUAAY) 6 wherein Y is C or U (SEQ ID NO:104) preferably the core QKI binding site is ACUAAC. A preferred oligonucleotide consists of or consists essentially of (ACUAAC) 6 (SEQ ID NO: 73).

Further preferred oligonucleotides derived from SEQ ID NO: 70, 71, 72 or 73 are later disclosed herein.

In a further embodiment, there is provided an oligonucleotide comprising a core QKI binding site UACUAAY wherein Y is C or U, preferably the core QKI binding site is UACUAAC. The length of such oligonucleotide may be from 17 to 40 nucleotides or any of 17, 18, 19, 20, 21, 22, 23, 24, 25, 26, 27, 28, 29, 30, 31, 32, 33, 34, 35, 36, 37, 38, 39, 40 nucleotides.

In a preferred embodiment, the oligonucleotide comprises, consists of or consists essentially of (UACUAAY) n wherein Y is C or U and n is an integer ranged from 1 to 6 (n=2-6 correspond to SEQ ID NO: 106-110 respectively) preferably the core QKI binding site is UACUAAC. The length of such oligonucleotide may be from 6 to 42 nucleotides or any of 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 nucleotides.

In a preferred embodiment, the oligonucleotide comprises, consists of or consists essentially of (UACUAAY) 1 wherein Y is C or U preferably the core QKI binding site is UACUAAC.

In a preferred embodiment, the oligonucleotide comprises, consists of or consists essentially of (UACUAAY) 2 wherein Y is C or U (SEQ ID NO: 106) preferably the core QKI binding site is UACUAAC. A preferred oligonucleotide consists of or consists essentially of (UACUAAC) 2 (SEQ ID NO: 94).

In a preferred embodiment, the oligonucleotide comprises, consists of or consists essentially of (UACUAAY) 3 wherein Y is C or U (SEQ ID NO:107) preferably the core QKI binding site is UACUAAC. A preferred oligonucleotide consists of or consists essentially of (UACUAAC) 3 (SEQ ID NO: 95).

In a preferred embodiment, the oligonucleotide comprises, consists of or consists essentially of (UACUAAY) 4 wherein Y is C or U (SEQ ID NO: 108) preferably the core QKI binding site is UACUAAC. A preferred oligonucleotide consists of or consists essentially of (UACUAAC) 4 (SEQ ID NO: 96).

In a preferred embodiment, the oligonucleotide comprises, consists of or consists essentially of (UACUAAY) 5 wherein Y is C or U (SEQ ID NO: 109) preferably the core QKI binding site is UACUAAC.

In a preferred embodiment, the oligonucleotide comprises, consists of or consists essentially of (UACUAAY) 6 wherein Y is C or U (SEQ ID NO:110) preferably the core QKI binding site is UACUAAC. A preferred oligonucleotide consists of or consists essentially of (UACUAAC) 6 (SEQ ID NO: 97).

Further preferred oligonucleotides derived from SEQ ID NO: 94, 95, 96, 97 are later disclosed herein.

The following embodiments apply for all core and half QKI binding sites defined earlier herein, especially UACUAAY as core QKI binding site, wherein Y is C or U and YAAY as half QKI binding site, wherein Y is C or U.

In an embodiment, when there is a half QKI binding site in the oligonucleotide as defined earlier herein, this half QKI binding site is present upstream/5′ side of the core QKI binding site. However, in another embodiment, when there is a half QKI binding site in the oligonucleotide as defined earlier herein, the half QKI binding site is present downstream/3′ side of the core QKI binding site. These embodiments apply for all core and half QKI binding sites defined earlier herein, especially UACUAAY as core QKI binding site, wherein Y is C or U and YAAY as half QKI binding site, wherein Y is C or U.

For oligonucleotides as described in this application, when a feature of a monomer is not defined and is not apparent from context, the corresponding feature from an RNA monomer is to be assumed. Preferably, said monomers are RNA monomers, or are derived from RNA monomers. In a preferred embodiment, the oligonucleotide of the invention is a single stranded oligonucleotide. This is attractive for the invention as the oligonucleotide should be able to inhibit an activity of a QKI protein as described earlier herein. This inhibition of an activity of a QKI protein via its binding to the QKI protein may be reversible and the protein should not be degraded. In an embodiment, the oligonucleotide of the invention is not double stranded. It may be expected that such a double stranded oligonucleotide may not bind a QKI protein and may not be able to inhibit an activity of said protein. It may be expected that such a double stranded oligonucleotide may not bind a QKI protein to the same extent as a single stranded oligonucleotide will do. It may be expected that such a double stranded oligonucleotide may not be able to inhibit an activity of said protein to the same extent as a single stranded oligonucleotide will do.

The most common naturally occurring nucleotides in RNA are adenosine monophosphate, cytidine monophosphate, guanosine monophosphate, thymidine monophosphate, and uridine monophosphate. These consist of a pentose sugar ribose, a 5′-linked phosphate group which is linked via a phosphate ester, and a 1′-linked base. The sugar connects the base and the phosphate, and is therefore often referred to as the scaffold of the nucleotide. A modification in the pentose sugar is therefore often referred to as a scaffold modification. A sugar modification may therefore be called a scaffold modification. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine, or uracil, or a derivative thereof. Cytosine, thymine, and uracil are pyrimidine bases, and are generally linked to the scaffold through their 1-nitrogen. Adenine and guanine are purine bases, and are generally linked to the scaffold through their 9-nitrogen. A base (or nucleobase) present in the oligonucleotide may be modified or substituted by another base. However, when at least one of the bases of said oligonucleotide base sequence is substituted by a different base, such different base should have the same or similar base pairing activity as the one initially identified in said base sequence.

A nucleotide is generally connected to neighbouring nucleotides through condensation of its 5′-phosphate moiety to the 3′-hydroxyl moiety of the neighbouring nucleotide monomer. Similarly, its 3′-hydroxyl moiety is generally connected to the 5′-phosphate of a neighbouring nucleotide monomer. This forms phosphodiester bonds. The phosphodiesters and the scaffold form an alternating copolymer. The bases are grafted to this copolymer, namely to the scaffold moieties. Because of this characteristic, the alternating copolymer formed by linked monomers of an oligonucleotide is often called the backbone of the oligonucleotide. Because the phosphodiester bonds connect neighbouring monomers together, they are often referred to as backbone linkages. It is understood that when a phosphate group is modified so that it is instead an analogous moiety such as a phosphorothioate, such a moiety is still referred to as the backbone linkage of the monomer. This is referred to as a backbone linkage modification. In general terms, the backbone of an oligonucleotide is thus comprised of alternating scaffolds and backbone linkages.

In an embodiment, the oligonucleotide is a modified RNA oligonucleotide. Such modified RNA oligonucleotide may comprising a nucleotide analogue and/or a modified internucleotide linkage. A “modified internucleotide linkage” may be replaced by the wording “backbone linkage modification” as explained earlier herein.

In an embodiment, the nucleotide analogue comprises a modified base and/or a modified sugar and/or wherein the modified internucleotide linkage. In an embodiment, the modified internucleotide linkage is a phosphorothioate internucleotide linkage.

In an embodiment, a base modification (or a modified base) can include a modified version of the natural purine and pyrimidine bases (e.g. adenine, uracil, guanine, cytosine, and thymine), such as hypoxanthine, pseudouracil, pseudocytosine, 1-methylpseudouracil, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-halomethyluracil, 5-trifluoromethyluracil, 5-propynyluracil, 5-propynylcytosine, 5-aminomethyluracil, 5-hydroxymethyluracil, 5-aminomethylcytosine, 5-methylcytosine, 5-methylcytidine, 5-hydroxymethylcytosine, Super T, or as described in e.g. Kumar et al. J. Org. Chem. 2014, 79, 5047; Leszczynska et al. Org. Biol. Chem. 2014, 12, 1052), pyrazolo[1,5-a]-1,3,5-triazine C-nucleoside (as in e.g. Lefoix et al. J. Org. Chem. 2014, 79, 3221), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, boronated cytosine (as in e.g. Nizioł et al. Bioorg. Med. Chem. 2014, 22, 3906), pseudoisocytidine, C (Pyc) (as in e.g. Yamada et al. Org. Biomol. Chem. 2014, 12, 2255) and N4-ethylcytosine, or derivatives thereof; N²-cyclopentylguanine (cPent-G), N²-cyclopentyl-2-aminopurine (cPent-AP), and N²-propyl-2-aminopurine (Pr-AP), carbohydrate-modified uracil (as in e.g. Kaura et al. Org. Lett. 2014, 16, 3308), amino acid modified uracil (as in e.g. Guenther et al. Chem. Commun. 2014, 50, 9007); or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173 (Epoch Biosciences), which is incorporated here entirely by reference. cPent-G, cPent-AP and Pr-AP were shown to reduce immunostimulatory effects when incorporated in siRNA (Peacock H. et al. J. Am. Chem. Soc. 2011, 133, 9200). Examples of modified bases are described in e.g. WO2014/093924 (ModeRNA).

A preferred modified base is 5-methylcytosine and 5-methylcytidine.

Depending on its length an oligonucleotide of the invention may comprise 1, 2, 3, 4, 5, 6, 7, 8, 9, 10 base modifications. It is also encompassed by the invention to introduce more than one distinct base modification in said oligonucleotide.

A modified sugar in a nucleotide of the oligonucleotide is synonymous of a scaffold modification of the oligonucleotide.

A scaffold modification can include a modified version of the ribosyl moiety, such as 2′-O-modified RNA such as 2′-O-alkyl or 2′-O-(substituted) alkyl e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-O-propargyl, 2′-O-acetalester (such as e.g. Biscans et al. Bioorg. Med. Chem. 2015, 23, 5360), 2′-O-allyl, 2′-O-(2S-methoxypropyl), 2′-O—(N-(aminoethyl) carbamoyl)methyl) (2′-AECM), 2′-O-(2-carboxyethyl) and carbamoyl derivatives (Yamada et al. Org. Biomol. Chem. 2014, 12, 6457), 2′-O-(2-amino) propyl, 2′-O-(2-(dimethylamino) propyl), 2′-O-(2-amino)ethyl, 2′-O-(2-(dimethylamino)ethyl); 2′-deoxy (DNA); 2′-O-(haloalkoxy)methyl (Arai K. et al. Bioorg. Med. Chem. 2011, 21, 6285) e.g. 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl] (MOCE), 2′-O-[2-(N-methylcarbamoyl)ethyl] (MCE), 2′-O-[2-(N,N-dimethylcarbamoyl)ethyl] (DCME), 2′-O-[2-(methylthio)ethyl] (2′-MTE), 2′-(ω-O-serinol); 2′-halo e.g. 2′-F, FANA (2′-F arabinosyl nucleic acid); 2′,4′-difluoro-2′-deoxy; carbasugar and azasugar modifications; 3′-O-substituted e.g. 3′-O-methyl, 3′-O-butyryl, 3′-O-propargyl; 4′-substituted e.g. 4′-aminomethyl-2′-O-methyl or 4′-aminomethyl-2′-fluoro; 5′-substituted e.g. 5′-methyl or CNA (Østergaard et al. ACS Chem. Biol. 2014, 22, 6227); and their derivatives.

A scaffold modification can include a bicyclic nucleic acid monomer (BNA) which may be a bridged nucleic acid monomer. Each occurrence of said BNA may result in a monomer that is independently chosen from the group consisting of a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted-2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNA^{NC} (N—H) monomer, a 2′,4′-BNA^{NC} (N-Me) monomer, a 2′,4′-BNA^{NC} (N-Bn) monomer, an ethylene-bridged nucleic acid (ENA) monomer, a carba LNA (cLNA) monomer, a 3,4-dihydro-2H-pyran nucleic acid (DpNA) monomer, a 2′-C-bridged bicyclic nucleotide (CBBN) monomer, a heterocyclic-bridged BNA monomer (such as triazolyl or tetrazolyl-linked), an amido-bridged BNA monomer, an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an oxetane nucleotide monomer, a locked PMO monomer derived from 2′-amino-LNA, a guanidine-bridged nucleic acid (GuNA) monomer, a spirocyclopropylene-bridged nucleic acid (scpBNA) monomer, and derivatives thereof.

A preferred sugar modification is selected from:
2′-O-modified RNA, more preferably 2′-O-alkyl or 2′-O-(substituted) alkyl, even more preferably 2′-O-methyl or 2′-O-(2-methoxy)ethyl (2′-MOE)
(BNA), more preferably a (CRN) monomer or a locked nucleic acid (LNA) monomer.

Another preferred sugar modification is selected from 2′-O-modified RNA, more preferably 2′-O-alkyl or 2′-O-(substituted) alkyl, even more preferably 2′-O-methyl or 2′-O-(2-methoxy)ethyl (2′-MOE)

More preferred sugar modification is 2′-O-methyl and a locked nucleic acid (LNA) monomer.

More preferred sugar modification is 2′-O-methyl.

If a LNA modification is present, it is not present in the spacer (or central part of the oligonucleotide). However it may be present in a wing of the oligonucleotide.

Depending on its length an oligonucleotide of the invention may comprise 1, 2, 3, 4, 5, 6, 7, 8, 9, 10 scaffold modifications. It is also encompassed by the invention to introduce more than one distinct scaffold modification in said oligonucleotide.

Oligonucleotides according to the invention can comprise backbone linkage modifications. A backbone linkage modification can be, but is not limited to, a modified version of the phosphodiester present in RNA, such as phosphorothioate (PS), chirally pure phosphorothioate, (R)-phosphorothioate, (S)-phopshorothioate, phosphorodithioate (PS2), phosphonoacetate (PACE), phosphonoacetamide (PACA), thiophosphonoacetate (thioPACE), thiophosphonoacetamide, phosphorothioate prodrug, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphate, phosphotriester, aminoalkylphosphotriester, and their derivatives. Another modification includes phosphoryl guanidine, phosphoramidite, phosphoramidate, N3′→P5′ phosphoramidate, phosphordiamidate, phosphorothiodiamidate, sulfamate, dimethylenesulfoxide, amide, sulfonate, siloxane, sulfide, sulfone, formacetyl, thioformacetyl, methylene formacetyl, alkenyl, methylenehydrazino, sulfonamide, amide, triazole, oxalyl, carbamate, methyleneimino (MMI), and thioacetamido nucleic acid (TANA); and their derivatives. Examples of chirally pure phosphorothioate linkages are described in e.g. WO2014/010250 or WO2017/062862 (WaVe Life Sciences). Examples of phosphoryl guanidine linkages are described in WO2016/028187 (Noogen). Various salts, mixed salts and free acid forms are also included, as well as 3′→3′ and 2′→5′ linkages.

A preferred backbone linkage modification is PS, PS2, phosphoramidate and phosphordiamidate.

Depending on its length, an oligonucleotide of the invention may comprise 1, 2, 3, 4, 5, 6, 7, 8, 9, 10 backbone linkage modifications. It is also encompassed by the invention to introduce more than one distinct backbone modification in said oligonucleotide.

It is preferred that the nucleotide and the internucleotide linkage of the core and if present of the half core QKI binding site are not modified and are therefore those normally found in RNA. It is further preferred that at least a nucleotide and/or at least an internucleotide linkage that is present at the 5′ and/or at the 3′ end of the core and if present of the half core QKI binding sites are modified. These embodiments apply for all core and half QKI binding sites defined earlier herein, UACUAAY as core QKI binding site, wherein Y is C or U and YAAY as half QKI binding site, wherein Y is C or U.
Modifications encompassed have been all defined herein. It is expected that modifying the oligonucleotide at such places may contribute to improve its stability or resistance to exonucleases. This is an advantage when the oligonucleotide is administrated as such to a patient (i.e. naked administration).
In an embodiment, the last 1, 2, 3, 4 nucleotides and/or internucleotide linkages at the 5′ of the oligonucleotide are modified.
In an embodiment, the last 1, 2, 3, 4 nucleotides and/or internucleotide linkages at the 3′ of the oligonucleotide are modified.
In an embodiment, the last 1, 2, 3, 4 nucleotides and/or internucleotide linkages at the 5′ and at the 3′ of the oligonucleotide are modified. Preferably, 2 nucleotides and/or internucleotide linkages at the 5′ and at the 3′ of the oligonucleotide are modified. Preferably, 4 nucleotides and/or internucleotide linkages at the 5′ and at the 3′ of the oligonucleotide are modified.
In an embodiment, the oligonucleotide is such that its backbone (i.e. internucleotide linkage) in its central part has not been modified and preferably wherein the internucleotide linkages at the 2 to 4 most 5′ end and 2 to 4 most 3′ end of the oligonucleotide have been modified.
In an embodiment, the oligonucleotide is such that its sugars in its central part have not been modified and preferably wherein its sugars or its nucleotides at the 2 to 4 most 5′ end and/or 2 to 4 most 3′ end of the oligonucleotide have been modified.
In an embodiment, the oligonucleotide is such that its backbone (i.e. internucleotide linkage) and sugars in its central part have not been modified and preferably wherein the internucleotide linkages and sugars or its nucleotides at the 2 to 4 most 5′ end and/or 2 to 4 most 3′ end of the oligonucleotide have been modified.
In an embodiment, when one refers to the modifications at the 5′ and/or 3′ end of the oligonucleotide (preferably at the 2 to 4 most 5′ end and/or 2 to 4 most 3′ end of the oligonucleotide), such modifications are:
the modified internucleotide linkage is PS and/or
the modified sugar is 2′-O-methyl
the modified base is 5-methylcytosine and/or
the modified nucleotide is a locked nucleic acid (LNA) monomer.
   Preferably, such modifications are:
the modified internucleotide linkage is PS and
the modified sugar is 2′-O-methyl.
   Preferably, such modifications are:
the modified internucleotide linkage is PS,
the modified sugar is 2′-O-methyl and
the modified base is 5-methylcytosine.
   Preferably, such modifications are:
the modified internucleotide linkage is PS and
the modified nucleotide is a locked nucleic acid (LNA) monomer.
   Preferably, such modifications are:
the modified internucleotide linkage is PS,
the modified nucleotide is a locked nucleic acid (LNA) monomer and
the modified base is 5-methylcytosine
   In an embodiment, the oligonucleotide (oligonucleotide 1) is as follows: GCUUUACUAACACAGUACUAACAUCG (SEQ ID NO:11), wherein the underlined nucleotides have a phosphorothioate linkage and all nucleotides have a 2′-O-methyl base. The corresponding non-modified oligonucleotides is represented by SEQ ID NO: 10.
   In another embodiment, the oligonucleotide (oligonucleotide 2) is as follows:
   GCUUUACUAACACUCACCUACUAACAUCG (SEQ ID NO:13), wherein the underlined nucleotides have a phosphorothioate linkage and all nucleotides have a 2′-O-methyl base. The corresponding non-modified oligonucleotides is represented by SEQ ID NO: 12.
   In another embodiment, the oligonucleotide (oligonucleotide 3) is as follows:
   GCCGUAACCACGUCUACUAACGCCG (SEQ ID NO:15), wherein the underlined nucleotides have a phosphorothioate linkage and all nucleotides have a 2′-O-methyl base. The corresponding non-modified oligonucleotides is represented by SEQ ID NO: 14.
   SEQ ID NO:10, 12 and 14 represent the sequence of the oligonucleotide identified above as non modified RNA. SEQ ID NO: 11, 13 and 15 represent the sequence of the oligonucleotide identified above as modified RNA.
   Further preferred oligonucleotides derived from SEQ ID NO: 59, 55, 57 or 61 are disclosed below:
   In an embodiment, the oligonucleotide comprises two core QKI binding sites that are separated by 3 nucleotides and it does not comprise a half QKI binding site. The length of such oligonucleotide may be from 17 to 40 nucleotides or any of 17, 18, 19, 20, 21, 22, 23, 24, 25, 26, 27, 28, 29, 30, 31, 32, 33, 34, 35, 36, 37, 38, 39, 40 nucleotides. A preferred oligonucleotide comprises or consists of or essentially consists of the base sequence SEQ ID NO:59. This oligonucleotide may be further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base.
   In an embodiment, the oligonucleotide comprises two core QKI binding sites that are separated by 4 nucleotides and it does not comprise a half QKI binding site. The length of such oligonucleotide may be from 18 to 40 nucleotides or any of 18, 19, 20, 21, 22, 23, 24, 25, 26, 27, 28, 29, 30, 31, 32, 33, 34, 35, 36, 37, 38, 39, 40 nucleotides. A preferred oligonucleotide comprises or consists of or essentially consists of the base sequence SEQ ID NO:55. This oligonucleotide may be further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base.
   In an embodiment, the oligonucleotide comprises two core QKI binding sites that are separated by 7 nucleotides and it does not comprise a half QKI binding site. The length of such oligonucleotide may be from 21 to 40 nucleotides or any of 21, 22, 23, 24, 25, 26, 27, 28, 29, 30, 31, 32, 33, 34, 35, 36, 37, 38, 39, 40 nucleotides. A preferred oligonucleotide comprises or consists of or essentially consists of the base sequence SEQ ID NO:57. This oligonucleotide may be further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base.
   In an embodiment, the oligonucleotide comprises two core QKI binding sites that are separated by 5 nucleotides and it does not comprise a half QKI binding site. The length of such oligonucleotide may be from 19 to 40 nucleotides or any of 19, 20, 21, 22, 23, 24, 25, 26, 27, 28, 29, 30, 31, 32, 33, 34, 35, 36, 37, 38, 39, 40 nucleotides. A preferred oligonucleotide comprises or consists of or essentially consists of the base sequence SEQ ID NO:61. This oligonucleotide may be further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base.
   A more preferred oligonucleotide comprises, consists or essentially consists of SEQ ID NO: 65.
   Further preferred oligonucleotides derived from SEQ ID NO: 70, 71, 72, 73 are disclosed below:
   In a preferred embodiment, the oligonucleotide comprises, consists of or consists essentially of (ACUAAY) n wherein Y is C or U and n is an integer ranged from 1 to 6 (n=2-6 correspond to SEQ ID NO: 100-104) (preferably the core QKI binding site is ACUAAC and the oligonucleotide is further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base. The length of such oligonucleotide may be from 6 to 40 nucleotides or any of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides.
   In a preferred embodiment, the oligonucleotide comprises, consists of or consists essentially of (ACUAAY) 1 wherein Y is C or U preferably the core QKI binding site is ACUAAC and the oligonucleotide is further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base.
   In a preferred embodiment, the oligonucleotide comprises, consists of or consists essentially of (ACUAAY) 2 wherein Y is C or U (SEQ ID NO: 100) preferably the core QKI binding site is ACUAAC and the oligonucleotide is further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base. A preferred oligonucleotide consists of or consists essentially of (ACUAAC) 2 (SEQ ID NO: 70) and is further modified as defined earlier in this paragraph. A more preferred oligonucleotide comprises, consists of or essentially consists of SEQ ID NO:74 or 90. SEQ ID NO:74 and 90 only differ by the presence of the C6 biotin in SEQ ID NO:74. In an embodiment, the oligonucleotide comprising SEQ ID NO:74 or 90 is further modified by not having all its nucleotides comprising a 2-O′-methyl base, or having some of its nucleotides comprising a deoxyribonucleic acid or comprising a TEG spacer (see for example SEQ ID NO:69).
   In a preferred embodiment, the oligonucleotide comprises, consists of or consists essentially of (ACUAAY) 3 wherein Y is C or U (SEQ ID NO: 101) preferably the core QKI binding site is ACUAAC and the oligonucleotide is further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base. A preferred oligonucleotide consists of or consists essentially of (ACUAAC) 3 (SEQ ID NO: 71) and is further modified as defined earlier in this paragraph. A more preferred oligonucleotide comprises, consists of or essentially consists of SEQ ID NO:75 or 91. SEQ ID NO:75 and 91 only differ by the presence of the C6 biotin in SEQ ID NO:75. In an embodiment, the oligonucleotide comprising SEQ ID NO:75 or 91 is further modified by not having all its nucleotides comprising a 2-O′-methyl base, or having some of its nucleotides comprising a deoxyribonucleic acid or comprising a TEG spacer (see for example SEQ ID NO:69).
   In a preferred embodiment, the oligonucleotide comprises, consists of or consists essentially of (ACUAAY) 4 wherein Y is C or U (SEQ ID NO: 102) preferably the core QKI binding site is ACUAAC and the oligonucleotide is further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base. A preferred oligonucleotide consists of or consists essentially of (ACUAAC) 4 (SEQ ID NO: 72) and is further modified as defined earlier in this paragraph. A more preferred oligonucleotide comprises, consists of or essentially consists of SEQ ID NO:76 or 92 or 79 or 114. SEQ ID NO:76 and 92 have all their sugars that comprise a 2-O′-methyl modification. SEQ ID NO: 76 and 92 only differ by the presence of the C6 biotin in SEQ ID NO:76.
   SEQ ID NO:79 and 114 have all their internucleotide linkages as phosphorothioate. SEQ ID NO: 79 and 114 only differ by the presence of the C6 biotin in SEQ ID NO:79.
   In a preferred embodiment, the oligonucleotide comprising SEQ ID NO:76 or 92 is further modified by not having all its nucleotides comprising a 2-O′-methyl base (preferably as SEQ ID NO:68 or 111), or having some of its nucleotides comprising a deoxyribonucleic acid (preferably as SEQ ID NO: 66 or 112) or comprising a TEG spacer (preferably as SEQ ID NO: 69 or 113). SEQ ID NO: 111 is identical with SEQ ID NO: 68 with the only difference that SEQ ID NO: 111 does not have the C6 biotin part. SEQ ID NO: 112 is identical with SEQ ID NO: 66 with the only difference that SEQ ID NO: 112 does not have the C6 biotin part. SEQ ID NO: 113 is identical with SEQ ID NO: 69 with the only difference that SEQ ID NO: 114 does not have the C6 biotin part.
   A preferred oligonucleotide comprises, consists of or consists essentially of (ACUAAC) 5 wherein Y is C or U (SEQ ID NO: 105) preferably the core QKI binding site is ACUAAC and the oligonucleotide is further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base. A preferred oligonucleotide consists of or consists essentially of (ACUAAC) 5 (SEQ ID NO: 105) and is further modified as defined earlier in this paragraph.
   In a preferred embodiment, the oligonucleotide comprises, consists of or consists essentially of (ACUAAY) 6 wherein Y is C or U (SEQ ID NO:104) preferably the core QKI binding site is ACUAAC and the oligonucleotide is further modified. It may comprise at least one phosphorothioate linkage and/or at least one nucleotide with a 2-O′-methyl base. In a preferred embodiment, all internucleotide linkages are phosphorothioate linkages and/or all nucleotides have a 2-O′-methyl base. A preferred oligonucleotide consists of or consists essentially of (ACUAAC) 6 (SEQ ID NO: 77) and is further modified as defined earlier in this paragraph. A more preferred oligonucleotide comprises, consists of or essentially consists of SEQ ID NO:77 or 93. SEQ ID NO:77 and 93 only differ by the presence of the C6 biotin in SEQ ID NO:77. In an embodiment, the oligonucleotide comprising SEQ ID NO:77 or 93 is further modified by not having all its nucleotides comprising a 2-O′-methyl base, or having some of its nucleotides comprising a deoxyribonucleic acid or comprising a TEG spacer (see for example SEQ ID NO:69).
   Further preferred oligonucleotides derived from SEQ ID NO: 94, 95, 96, 97 are later disclosed herein.
   In a preferred embodiment, the oligonucleotide comprises, consists of or consists essentially of (UACUAAY) 2 wherein Y is C or U (SEQ ID NO: 106) preferably the core QKI binding site is UACUAAC and the oligonucleotide is further modified. A preferred oligonucleotide consists of or consists essentially of (UACUAAC) 2 (SEQ ID NO:94) and said oligonucleotide is further modified. It may comprise at least one phosphorothioate linkage and/or at least one nucleotide with a 2-O′-methyl base and/or at least one deoxyribonucleic acid and/or a TEG spacer (see for example SEQ ID NO: 69). In a preferred embodiment, all internucleotide linkages are phosphorothioate linkages and/or all nucleotides have a 2-O′-methyl base.
   In a preferred embodiment, the oligonucleotide comprises, consists of or consists essentially of (UACUAAY) 3 wherein Y is C or U (SEQ ID NO: 107) preferably the core QKI binding site is UACUAAC and the oligonucleotide is further modified. A preferred oligonucleotide consists of or consists essentially of (UACUAAC) 3 (SEQ ID NO:95) and said oligonucleotide is further modified. It may comprise at least one phosphorothioate linkage and/or at least one nucleotide with a 2-O′-methyl base and/or at least one deoxyribonucleic acid and/or a TEG spacer (see for example SEQ ID NO: 69). In a preferred embodiment, all internucleotide linkages are phosphorothioate linkages and/or all nucleotides have a 2-O′-methyl base.
   In a preferred embodiment, the oligonucleotide comprises, consists of or consists essentially of (UACUAAY) 4 wherein Y is C or U (SEQ ID NO: 108) preferably the core QKI binding site is UACUAAC and the oligonucleotide is further modified. A preferred oligonucleotide consists of or consists essentially of (UACUAAC) 4 (SEQ ID NO: 96) and said oligonucleotide is further modified. It may comprise at least one phosphorothioate linkage and/or at least one nucleotide with a 2-O′-methyl base and/or at least one deoxyribonucleic acid and/or a TEG spacer (see for example SEQ ID NO: 69). In a preferred embodiment, all internucleotide linkages are phosphorothioate linkages and/or all nucleotides have a 2-O′-methyl base.
   In a preferred embodiment, the oligonucleotide comprises, consists of or consists essentially of (UACUAAY) 5 wherein Y is C or U (SEQ ID NO:109) preferably the core QKI binding site is UACUAAC and the oligonucleotide is further modified. It may comprise at least one phosphorothioate linkage and/or at least one nucleotide with a 2-O′-methyl base and/or at least one deoxyribonucleic acid and/or a TEG spacer (see for example SEQ ID NO: 69). In a preferred embodiment, all internucleotide linkages are phosphorothioate linkages and/or all nucleotides have a 2-O′-methyl base.
   In a preferred embodiment, the oligonucleotide comprises, consists of or consists essentially of (UACUAAY) 6 wherein Y is C or U (SEQ ID NO: 110) preferably the core QKI binding site is UACUAAC and the oligonucleotide is further modified. A preferred oligonucleotide consists of or consists essentially of (UACUAAC) 6 (SEQ ID NO: 97) and said oligonucleotide has been further modified. It may comprise at least one phosphorothioate linkage and/or at least one nucleotide with a 2-O′-methyl base and/or at least one deoxyribonucleic acid and/or a TEG spacer (see for example SEQ ID NO: 69). In a preferred embodiment, all internucleotide linkages are phosphorothioate linkages and/or all nucleotides have a 2-O′-methyl base.
   In a preferred embodiment, the oligonucleotide comprises, consists of or essentially consists of SEQ ID NO: 10, 11, 12, 13, 14, 15, 55, 57, 59, 61, 63, 65, 66, 68, 69, 70, 71, 72, 73, 74, 78, 79, 81, 82, 90, 91, 92, 93, 94, 95, 96, 97, 98, 99, 100, 101, 102, 103, 104, 105, 106, 107, 108, 109,110, 111, 112, 113, 114.

### Nucleic Acid Construct/Expression Vector/Viral Vector

In a further aspect a nucleic acid construct is provided comprising a nucleic acid sequence encoding the oligonucleotide of the invention. The oligonucleotide of the invention has been earlier defined herein. A “nucleic acid construct” as described herein has its customary and ordinary meaning as understood by one of skill in the art in view of this disclosure. A “nucleic acid construct” comprises a nucleic acid sequence encoding the oligonucleotide of the invention. Usually said nucleic acid sequence is operatively linked to a promoter that controls its expression. The part of this application entitled “general information” comprises more detail as to a “nucleic acid construct”. “Operatively linked” as used herein is further described in the part of this application entitled “general information”.
In some embodiments, a nucleic acid construct as described herein is suitable for expression in a mammal. As used herein, “suitable for expression in a mammal” may mean that the nucleic acid construct includes one or more regulatory sequences, selected on the basis of the mammalian host cells to be used for expression, operatively linked to the nucleotide sequence to be expressed. Preferably, said mammalian host cells to be used for expression are human or murine cells.
Additional sequences may be present in the nucleic acid construct of the invention. Exemplary additional sequences suitable herein include inverted terminal repeats (ITRs), Within the context of the invention, “ITRs” is intended to encompass one 5′ITR and one 3′ITR, each being derived from the genome of an AAV. Preferred ITRs are from AAV2.
Nucleic acid constructs described herein can be placed in expression vectors. Thus, in another aspect there is provided an expression vector comprising a nucleic acid construct as described herein.
A description of “expression vector” has been provided under the section entitled “general information”.
In some embodiments, the expression vector is a viral expression vector or viral vector. Therefore, in a further aspect of the invention, there is provided a viral vector comprising a nucleic acid sequence encoding the oligonucleotide of the invention. The oligonucleotide of the invention has been earlier defined herein. It is obvious to the skilled person that in said aspect, the nucleic acid sequence (DNA) codes for the oligonucleotide of the invention. In this aspect, the oligonucleotide of the invention has not been modified and is RNA. In this aspect, the viral vector may be administrated to a subject and not the oligonucleotide per se.
In some embodiments, a viral vector may be a viral vector selected from the group consisting of adenoviral vectors, adeno-associated viral vectors, retroviral vectors and lentiviral vectors. A preferred viral vector is an adeno-associated viral vector.
A description of “viral expression vector” has been provided under the section entitled “general information”. An adenoviral vector is also known as an adenovirus derived vector, an adeno-associated viral vector is also known as an adeno-associated virus derived vector, a retroviral vector is also known as a retrovirus derived vector and a lentiviral vector is also known as a lentivirus derived vector. A preferred viral vector is an adeno-associated viral vector. A description of “adeno-associated viral vector” has been provided under the section entitled “general information”.
In some embodiments, the vector is an adeno-associated vector or adeno-associated viral vector or an adeno-associated virus derived vector (AAV) selected from the group consisting of AAV of serotype 1 (AAV1), AAV of serotype 2 (AAV2), AAV of serotype 3 (AAV3), AAV of serotype 4 (AAV4), AAV of serotype 5 (AAV5), AAV of serotype 6 (AAV6), AAV of serotype 7 (AAV7), AAV of serotype 8 (AAV8), AAV of serotype 9 (AAV9), AAV of serotype rh10 (AAVrh10), AAV of serotype rh8 (AAVrh8), AAV of serotype Cb4 (AAVCb4), AAV of serotype rh74 (AAVrh74), AAV of serotype DJ (AAVDJ), AAV of serotype 2/5 (AAV2/5), AAV of serotype 2/1 (AAV2/1), AAV of serotype 1/2 (AAV1/2), AAV of serotype Anc80 (AAVAnc80). In a preferred embodiment, an AAV of serotype 2 is used.

### Composition

In an aspect of the invention is provided a composition comprising at least one oligonucleotide according to the invention, preferably wherein said composition comprises at least one excipient, and/or wherein said oligonucleotide comprises at least one conjugated ligand, that may further aid in enhancing the targeting and/or delivery of said composition and/or said oligonucleotide to a tissue and/or cell and/or into a tissue and/or cell.
In this aspect of the invention is also provided a composition comprising a viral vector according to the invention, preferably wherein said composition comprises at least one excipient that may further aid in enhancing the targeting and/or delivery of said composition and/or said viral vector to a tissue and/or cell and/or into a tissue and/or cell.
Compositions as described here are herein referred to as compositions according to the invention. A composition according to the invention can comprise one or more than one oligonucleotide according to the invention. In the context of this invention, an excipient can be a distinct molecule, but it can also be a conjugated moiety. In the first case, an excipient can be a filler, such as starch. In the latter case, an excipient can for example be a targeting ligand that is linked to the oligonucleotide according to the invention.
In a preferred embodiment, said composition is for use as a medicament. In a preferred embodiment, the same holds for the oligonucleotide of the invention. Said composition is therefore a pharmaceutical composition. A pharmaceutical composition usually comprises a pharmaceutically accepted carrier, diluent and/or excipient. In a preferred embodiment, a composition of the current invention comprises an oligonucleotide as defined herein and optionally further comprises a pharmaceutically acceptable formulation, filler, preservative, solubilizer, carrier, diluent, excipient, salt, adjuvant and/or solvent. Such pharmaceutically acceptable carrier, filler, preservative, solubilizer, diluent, salt, adjuvant, solvent and/or excipient may for instance be found in Remington: The Science and Practice of Pharmacy, 20th Edition. Baltimore, MD: Lippincott Williams & Wilkins, 2000

A pharmaceutical composition may comprise an aid in enhancing the stability, solubility, absorption, bioavailability, activity, pharmacokinetics, pharmacodynamics, cellular uptake, and intracellular trafficking of said compound, in particular an excipient capable of forming complexes, nanoparticles, microparticles, nanotubes, nanogels, hydrogels, poloxamers or pluronics, polymersomes, colloids, microbubbles, vesicles, micelles, lipoplexes, and/or liposomes. Examples of nanoparticles include polymeric nanoparticles, (mixed) metal nanoparticles, carbon nanoparticles, gold nanoparticles, magnetic nanoparticles, silica nanoparticles, lipid nanoparticles, sugar particles, protein nanoparticles and peptide nanoparticles. An example of the combination of nanoparticles and oligonucleotides is spherical nucleic acid (SNA), as in e.g. Barnaby et al. Cancer Treat. Res. 2015, 166, 23.

A preferred composition comprises at least one excipient that may further aid in enhancing the targeting and/or delivery of said composition and/or said oligonucleotide to a tissue and/or a cell and/or into a tissue and/or a cell. A preferred tissue or cell is the liver or the kidney or liver cells or kidney cells.

Many of these excipients are known in the art (e.g. see Bruno, 2011) and may be categorized as a first type of excipient. Examples of first type of excipients include polymers (e.g. polyethyleneimine (PEI), polypropyleneimine (PPI), dextran derivatives, butylcyanoacrylate (PBCA), hexylcyanoacrylate (PHCA), poly (lactic-co-glycolic acid) (PLGA), polyamines (e.g. spermine, spermidine, putrescine, cadaverine), chitosan, poly (amido amines) (PAMAM), poly (ester amine), polyvinyl ether, polyvinyl pyrrolidone (PVP), polyethylene glycol (PEG) cyclodextrins, hyaluronic acid, colominic acid, and derivatives thereof), dendrimers (e.g. poly (amidoamine)), lipids {e.g. 1,2-dioleoyl-3-dimethylammonium propane (DODAP), dioleoyldimethylammonium chloride (DODAC), phosphatidylcholine derivatives [e.g 1,2-distearoyl-sn-glycero-3-phosphocholine (DSPC)], lyso-phosphatidylcholine derivaties [e.g. 1-stearoyl-2-lyso-sn-glycero-3-phosphocholine (S-LysoPC)], sphingomyeline, 2-{3-[Bis-(3-amino-propyl)-amino]-propylamino}-N-ditetracedyl carbamoyl methylacetamide (RPR209120), phosphoglycerol derivatives [e.g. 1,2-dipalmitoyl-sn-glycero-3-phosphoglycerol, sodium salt (DPPG-Na), phosphaticid acid derivatives [1,2-distearoyl-sn-glycero-3-phosphaticid sodium acid, salt (DSPA), phosphatidylethanolamine derivatives [e.g. dioleoyl-L-R-phosphatidylethanolamine (DOPE), 1,2-distearoyl-sn-glycero-3-phosphoethanolamine (DSPE), 2-diphytanoyl-sn-glycero-3-phosphoethanolamine (DPhyPE),], N-[1-(2,3-dioleoyloxy) propyl]-N, N, N-trimethylammonium (DOTAP), N-[1-(2,3-dioleyloxy) propyl]-N, N, N-trimethylammonium (DOTMA), 1,3-di-oleoyloxy-2-(6-carboxy-spermyl)-propylamid (DOSPER), (1,2-dimyristyolxypropyl-3-dimethylhydroxy ethyl ammonium (DMRIE), (N1-cholesteryloxycarbonyl-3,7-diazanonane-1,9-diamine (CDAN), dimethyldioctadecylammonium bromide (DDAB), 1-palmitoyl-2-oleoyl-sn-glycerol-3-phosphocholine (POPC), (b-L-Arginyl-2,3-L-diaminopropionic acid-N-palmityl-N-olelyl-amide trihydrochloride (AtuFECT01), N,N-dimethyl-3-aminopropane derivatives [e.g. 1,2-distearoyloxy-N,N-dimethyl-3-aminopropane (DSDMA), 1,2-dioleyloxy-N,N-dimethyl-3-aminopropane (DoDMA), 1,2-Dilinoleyloxy-N, N-3-dimethylaminopropane (DLinDMA), 2,2-dilinoleyl-4-dimethylaminomethyl [1,3]-dioxolane (DLin-K-DMA), phosphatidylserine derivatives [1,2-dioleyl-sn-glycero-3-phospho-L-serine, sodium salt (DOPS)], proteins (e.g. albumin, gelatins, atellocollagen), and linear or cyclic peptides (e.g. protamine, PepFects, NickFects, polyarginine, polylysine, CADY, MPG, cell-penetrating peptides (CPPs), targeting peptides, cell-translocating peptides, endosomal escape peptides). Examples of such peptides have been described, e.g. muscle targeting peptides (e.g. Jirka et al., Nucl. Acid Ther. 2014, 24, 25), CPPs (e.g. Pip series, including WO2013/030569, and oligoarginine series, e.g. U.S. Pat. No. 9,161,948 (Sarepta), WO2016/187425 (Sarepta), and M12 peptide in e.g. Gao et al., Mol. Ther. 2014, 22, 1333), or blood-brain barrier (BBB) crossing peptides such as (branched) ApoE derivatives (Shabanpoor et al., Nucl. Acids Ther. 2017, 27, 130). Carbohydrates and carbohydrate clusters as described below, when used as distinct compounds, are also suitable for use as a first type of excipient.

Another preferred composition may comprise at least one excipient categorized as a second type of excipient. A second type of excipient may comprise or contain a conjugate group as described herein to enhance targeting and/or delivery of the composition and/or of the oligonucleotide of the invention to a tissue and/or cell and/or into a tissue and/or cell, as for example liver or kidney tissue or cell. The conjugate group may display one or more different or identical ligands. Examples of conjugate group ligands are e.g. peptides, vitamins, aptamers, carbohydrates or mixtures of carbohydrates (Han et al., Nature Communications, 2016, doi: 10.1038/ncomms10981; Cao et al., Mol. Ther. Nucleic Acids, 2016, doi: 10.1038/mtna.2016.46), proteins, small molecules, antibodies, polymers, drugs. Examples of carbohydrate conjugate group ligands are glucose, mannose, galactose, maltose, fructose, N-acetylgalactosamine (GalNac), glucosamine, N-acetylglucosamine, glucose-6-phosphate, mannose-6-phosphate, and maltotriose. Carbohydrates may be present in plurality, for example as end groups on dendritic or branched linker moieties that link the carbohydrates to the component of the composition. A carbohydrate can also be comprised in a carbohydrate cluster portion, such as a GalNAc cluster portion. A carbohydrate cluster portion can comprise a targeting moiety and, optionally, a conjugate linker. In some embodiments, the carbohydrate cluster portion comprises 1, 2, 3, 4, 5, 6, or more GalNAc groups. As used herein, “carbohydrate cluster” means a compound having one or more carbohydrate residues attached to a scaffold or linker group, (see, e.g., Maier et al., “Synthesis of Antisense Oligonucleotides Conjugated to a Multivalent Carbohydrate Cluster for Cellular Targeting,” Bioconjugate Chem., 2003, (14): 18-29; Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, (47): 5798-5808). In this context, “modified carbohydrate” means any carbohydrate having one or more chemical modifications relative to naturally occurring carbohydrates. As used herein, “carbohydrate derivative” means any compound which may be synthesized using a carbohydrate as a starting material or intermediate. As used herein, “carbohydrate” means a naturally occurring carbohydrate, a modified carbohydrate, or a carbohydrate derivative. Both types of excipients may be combined together into one single composition as identified herein. An example of a trivalent N-acetylglucosamine cluster is described in WO2017/062862 (Wave Life Sciences), which also describes a cluster of sulfonamide small molecules. An example of a single conjugate of the small molecule sertraline has also been described (Ferrés-Coy et al., Mol. Psych. 2016, 21, 328), as well as conjugates of protein-binding small molecules, including ibuprofen (e.g. U.S. Pat. No. 6,656,730 ISIS/Ionis Pharmaceuticals), spermine (e.g. Noir et al., J. Am. Chem Soc. 2008, 130, 13500), anisamide (e.g. Nakagawa et al., J. Am. Chem. Soc. 2010, 132, 8848) and folate (e.g. Dohmen et al., Mol. Ther. Nucl. Acids 2012, 1, e7). In an embodiment, the oligonucleotide is conjugated to lithocholic acid or eicosapentanoic acid.

In an embodiment, the oligonucleotide of the invention is conjugated to a peptide, vitamin, aptamer, carbohydrate or mixtures of carbohydrates, protein, small molecule, antibody, polymer, drug, lithocholic acid, eicosapentanoic acid or a cholesterol moeity. More preferably, the conjugation has been done at its 5′ or 3′ end. Even more preferably at its 3′ end.
In a preferred embodiment, the oligonucleotide of the invention is conjugated to a GalNac moiety.
More preferably, the conjugation has been done at it's 5′ or 3′ end.
It is also encompassed to conjugate the oligonucleotide of the invention to a cholesterol moiety at its 3′ end and to a GalNac moiety at its 5′ end.

Conjugates of oligonucleotides with aptamers are known in the art (e.g. Zhao et al. Biomaterials 2015, 67, 42).

Antibodies and antibody fragments can also be conjugated to an oligonucleotide of the invention. In a preferred embodiment, an antibody or fragment thereof targeting tissues of specific interest, particularly liver or kidney tissue, is conjugated to an oligonucleotide of the invention. Examples of such antibodies and/or fragments are e.g. targeted against CD71 (transferrin receptor), described in e.g. WO2016/179257 (CytoMx) and in Sugo et al. J. Control. Rel. 2016, 237, 1, or against equilibrative nucleoside transporter (ENT), such as the 3E10 antibody, as described in e.g. Weisbart et al., Mol. Cancer Ther. 2012, 11, 1.

In a preferred embodiment, the oligonucleotide of the invention is conjugated to a small molecule, aptamer or antibody. either at the 5′ or 3′ end. More preferably, the conjugation has been done at it's 5′ or 3′ end.

Other oligonucleotide conjugates are known to those skilled in the art, and have been reviewed in e.g. Winkler et al., Ther. Deliv. 2013, 4, 791, Manoharan, Antisense Nucl. Acid. Dev. 2004, 12, 103 and Ming et al., Adv. Drug Deliv. Rev. 2015, 87, 81.

The skilled person may select, combine and/or adapt one or more of the above or other alternative excipients and delivery systems to formulate and deliver an oligonucleotide for use in the present invention.

Such a pharmaceutical composition of the invention may be administered in an effective concentration at set times to an animal, preferably a mammal. More preferred mammal is a human being. An oligonucleotide or a composition as defined herein for use according to the invention may be suitable for direct administration to a cell, tissue and/or an organ in vivo of individuals affected by or at risk of developing a disease or condition as identified herein, and may be administered directly in vivo, ex vivo or in vitro. Administration may be via topical, systemic and/or parenteral routes, for example intravenous, subcutaneous, intraperitoneal, intrathecal, intramuscular, ocular, nasal, urogenital, intradermal, dermal, enteral, intravitreal, intracavernous, intracerebral, intrathecal, epidural or oral route.

Preferably, such a pharmaceutical composition or oligonucleotide of the invention may be encapsulated in the form of an emulsion, suspension, pill, tablet, capsule or soft-gel for oral delivery, or in the form of aerosol or dry powder for delivery to the respiratory tract and lungs.

In an embodiment an oligonucleotide of the invention may be used together with another compound already known to be used for the treatment of said disease.

Such combined use may be a sequential use: each component is administered in a distinct fashion, perhaps as a distinct composition. Alternatively each compound may be used together in a single composition.

Compounds that are comprised in a composition according to the invention can also be provided separately, for example to allow sequential administration of the active components of the composition according to the invention. In such a case, the composition according to the invention is a combination of compounds comprising at least an oligonucleotide according to the invention with or without a conjugated ligand and with at least one excipient as described above.

### Oligonucleotide/Viral Vector/Composition for Use

Compounds (oligonucleotide, viral vector) or compositions according to this invention are preferably for use as a medicament.
In an embodiment, the medicament is for treating a disease or condition associated with an elevated expression level of QKI. In an embodiment, the medicament is for treating a disease or condition associated with elevated expression level of QKI-5, QKI-6 and/or QKI-7. in an embodiment, the medicament is for treating a disease or condition associated with elevated expression level of QKI-5 and/or QKI-6.
The amino acid sequence of human QKI-5 is represented by SEQ ID NO: 16. A corresponding DNA coding sequence is represented by SEQ ID NO:17.
The amino acid sequence of human QKI-6 is represented by SEQ ID NO: 18. A corresponding DNA coding sequence is represented by SEQ ID NO:19.
The amino acid sequence of human QKI-7 is represented by SEQ ID NO: 20. A corresponding DNA coding sequence is represented by SEQ ID NO:21.
An elevated expression level of QKI may be assessed by comparison to the QKI expression level of a control healthy subject. QKI may be replaced with QKI-5, QKI-6 and/or QKI-7. In an embodiment, QKI is replaced with QKI-5 and/or QKI-6. In an embodiment, an elevated expression level means an elevation of at least 5% of the expression level. Preferably, an elevation means at least 10%, even more preferably at least 20%, at least 30%, at least 40%, at least 50%, at least 70%, at least 80%, at least 90%, or 100%. In an embodiment, expression may be assessed using a circRNA and assessing the expression in urine or in urine-derived cells. Expression level may be assessed by PCR or by Northern Blot.
In a preferred embodiment, a disease or condition associated with an elevated expression level of QKI is an inflammatory disease or condition.
Such inflammatory disease or condition may be selected from fibrosis, including in organs such as;
1) kidney: including but not exclusive to kidney injury such as kidney injury following ischemia reperfusion injury, acute kidney injury, chronic kidney injury, viral infections of the kidney;
2) lung: including but not exclusive to lung injury such as chronic obstructive pulmonary disease, idiopathic pulmonary fibrosis, pulmonary hypertension, bronchopulmonary dysplasia (BPD) or neonatal chronic lung disease;
3) heart: including but not exclusive to diastolic dysfunction, heart failure with preserved ejection fraction, arrythmias;
4) skin: including but not exclusive to scleroderma, keloid (scarring);
5) liver: including but not exclusive to autoimmune hepatitis, biliary obstruction, viral infections of the liver, iron overload, nonalcoholic fatty liver disease (NAFL) and nonalcoholic steatohepatitis (NASH); and
6) eye: including but not exclusive to conjunctival fibrosis and subretinal fibrosis.
   In an embodiment, fibrosis may occur in any organ such as kidney, liver, heart, lungs, skin and eyes. In an embodiment, an oligonucleotide or a viral vector or a composition for use in the invention is able to induce a therapeutic activity, effect, result in such disease or condition. The induction of such a therapeutic activity, effect, result may be assessed in vitro (i.e. cell free or in a cell) or in vivo (i.e. in an animal such as an animal model or in a patient). The induction of such a therapeutic activity, effect, result may be assessed at the molecular level and/or at the cellular level. It is also encompassed that the induction of such a therapeutic activity, effect, result improves or alleviates a parameter or symptom associated with such disease or condition.
   Within the context of the invention, the induction of a therapeutic activity, effect, result may be in at least one of:
The binding of QKI (i.e. binding of QKI-5, QKI-6 and/or QKI-7, preferably QKI-5 and/or QKI-6),
The inhibition of a QKI activity (i.e. binding of QKI-5, QKI-6 and/or QKI-7, preferably QKI-5 and/or QKI-6),
The decrease of QKI expression (i.e. QKI-5, QKI-6 and/or QKI-7 expression, preferably QKI-5 and/or QKI-6), The improvement of the expression level of a molecular marker associated with said disease or condition,
The improvement of a cellular effect associated with disease or condition,
The improvement or alleviation of a parameter or symptom associated with such disease or condition.
   This activity, effect, result is assessed by comparison to the level of these parameters or symptoms at the onset of the treatment.
   Binding of QKI and inhibition of a QKI activity have already been defined herein.
   The decrease of QKI expression (i.e. QKI-5, QKI-6 and/or QKI-7 expression, preferably QKI-5 and/or QKI-6) may be a decrease in the expression level of at least 5% of the expression level initial.
   Preferably, a decrease means at least 10%, even more preferably at least 20%, at least 30%, at least 40%, at least 50%, at least 70%, at least 80%, at least 90%, or 100%. Expression level may be assessed by PCR, Northern Blot, Western Blot or by Immunohistochemistry. In this latter case, it can be the case that there is no longer a detectable value associated with the parameter. The assessment of the inhibition of a QKI activity has been earlier defined herein.
   A molecular marker associated with said disease or condition may be any other relevant extracellular matrix/connective tissue marker that is associated with fibrosis. Such markers include proteoglycans (such as heparin sulfate, chondroitin sulfate, keratin sulfate), hyaluronic acid, a collagen (all types of collagens are encompassed), elastin, fibronectin and laminin. Collagen 3A is encompassed in an embodiment. TGFbeta is also encompassed as such a marker. TGFbeta is a key injury marker produced by damaged tubule cells of the kidney. Such marker may drive fibroblasts to produce extracellular matrix and may be linked with stimulation of endothelial to mesenchymal transition in damaged tissues/organs.
   The improvement of the expression level of a molecular marker associated with said disease or condition may mean the decrease of TGFbeta. The decrease of TGFbeta expression may be a decrease in the expression level of at least 5% of the expression level initial. Preferably, a decrease means at least 10%, even more preferably at least 20%, at least 30%, at least 40%, at least 50%, at least 70%, at least 80%, at least 90%, or 100%. Expression level may be assessed by PCR or by Northern Blot. In this latter case, it can be the case that there is no longer a detectable value associated with the parameter.
   The improvement of the expression level of a molecular marker associated with said disease or condition may mean the decrease of Collagen 3A. The decrease of Collagen 3A expression may be a decrease in the expression level of at least 5% of the expression level initial. Preferably, a decrease means at least 10%, even more preferably at least 20%, at least 30%, at least 40%, at least 50%, at least 70%, at least 80%, at least 90%, or 100%. Expression level may be assessed by PCR or by Northern Blot or by Immunohistochemistry. In this latter case, it can be the case that there is no longer a detectable value associated with the parameter.
   The improvement of a cellular effect associated with disease or condition may be the decrease of monocyte infiltration and macrophage chemotaxis. The decrease of monocyte infiltration and macrophage chemotaxis may be a decrease of at least 5% of the initial number of cells. Preferably, a decrease means at least 10%, even more preferably at least 20%, at least 30%, at least 40%, at least 50%, at least 70%, at least 80%, at least 90%, or 100%. The number of infiltrated monocytes and macrophage chemotaxis may be assessed using techniques known to the skilled person. Immunohistochemistry may be used, preferably as had been carried out in the experimental part.
   The improvement or alleviation of a parameter or symptom associated with such disease or condition may mean reducing the rate of increase or worsening of one or more of said symptoms. The improvement or alleviation of a parameter or symptom associated with such disease or condition may mean alleviating one or more characteristics of a diseased cell from a patient.
   All in all, the improvement or alleviation of a parameter or symptom associated with such disease or condition may mean the:
Attenuation of septal thickness in lung cells,
Reverse ventricular hypertrophy in lung cells,
Protection of organ injury (in any organ)
Reduction of neutrophil infiltration in any organ,
Reduction of monocyte infiltration in any organ,
Reduction of extracellular matrix deposition in any organ.

### Use/Method

In a further aspect, there is provided the use of a composition or of an oligonucleotide or of a viral vector as described in the previous sections for use as a medicament or part of therapy, or applications in which said oligonucleotide exerts its activity.

Preferably, an oligonucleotide or viral vector or composition of the invention is for use as a medicament or part of a therapy for preventing, delaying, curing, ameliorating and/or treating a disease or condition associated with an elevated expression level of QKI. In an embodiment, the disease or condition is an inflammatory disease or condition.

In an embodiment of this aspect of the invention is provided the oligonucleotide according to the invention, or the viral vector according to the invention, or the composition according to the invention, for use as a medicament, preferably for treating, preventing, and/or delaying a disease or condition associated with an elevated expression level of QKI (i.e. QKI-5, QKI-6 and/or QKI-7, preferably QKI-5 and/or QKI-6). In an embodiment, the disease or condition is an inflammatory disease or condition.

In a further aspect, there is provided a method for preventing, treating, curing, ameliorating and/or delaying a condition or disease as defined in the previous section in an individual, in a cell, tissue or organ of said individual. The method comprises administering an oligonucleotide or a viral vector or a composition of the invention to said individual or a subject in the need thereof.

The method according to the invention wherein an oligonucleotide or a composition as defined herein may be suitable for administration to a cell, tissue and/or an organ in vivo of individuals affected by any of the herein defined diseases or at risk of developing it, and may be administered in vivo, ex vivo or in vitro. An individual or a subject in need is preferably a mammal, more preferably a human being. Alternately, a subject is not a human. Administration may be via topical, systemic and/or parenteral routes, for example intravenous, subcutaneous, nasal, ocular, intraperitoneal, intrathecal, intramuscular, intracavernous, urogenital, intradermal, dermal, enteral, intravitreal, intracerebral, intrathecal, epidural or oral route.

In an embodiment, in a method of the invention, a concentration of an oligonucleotide or composition is ranged from 0.01 nM to 1 μM. More preferably, the concentration used is from 0.05 to 500 nM, or from 0.1 to 500 nM, or from 0.02 to 500 nM, or from 0.05 to 500 nM, even more preferably from 1 to 200 nM.

Dose ranges of an oligonucleotide or composition according to the invention are preferably designed on the basis of rising dose studies in clinical trials (in vivo use) for which rigorous protocol requirements exist. An oligonucleotide as defined herein may be used at a dose which is ranged from 0.01 to 200 mg/kg or 0.05 to 100 mg/kg or 0.1 to 50 mg/kg or 0.1 to 20 mg/kg, preferably from 0.5 to 10 mg/kg.

The ranges of concentration or dose of oligonucleotide or composition as given above are preferred concentrations or doses for in vitro or ex vivo uses. The skilled person will understand that depending on the identity of the oligonucleotide used, the target cell to be treated, the medium used and the transfection and incubation conditions, the concentration or dose of oligonucleotide used may further vary and may need to be optimised any further.

In an embodiment of this aspect of the invention is provided a method for preventing, treating, and/or delaying a disease or condition associated with an elevated expression level of QKI comprising administering to a subject an oligonucleotide according to the invention, a viral vector or a composition according to the invention. In an embodiment, the disease or condition is an inflammatory disease or condition

### General Definitions

Unless stated otherwise, all technical and scientific terms used herein have the same meaning as customarily and ordinarily understood by a person of ordinary skill in the art to which this invention belongs, and read in view of this disclosure.

### Gene or Coding Sequence

A “nucleic acid|” is represented by a nucleic acid sequence” which is a sequence of nucleotides in DNA or RNA that codes for a molecule that has a function. A nucleic acid sequence may comprise “non-coding sequence” as well as “coding sequence”. In the context of the application, a nucleic acid sequence is a non-coding sequence. Such a non-coding sequence may be the oligonucleotide of the invention.

### Promoter

As used herein, the term “promoter” or “transcription regulatory sequence” refers to a nucleic acid fragment that functions to control the transcription of one or more nucleic acid sequences, and is located upstream with respect to the direction of transcription of the transcription initiation site of said sequence.

### Operably Linked

As used herein, the term “operably linked” refers to a linkage of polynucleotide elements in a functional relationship. A nucleic acid is “operably linked” when it is placed into a functional relationship with another nucleic acid molecule. For instance, a transcription regulatory sequence is operably linked to a coding sequence if it affects the transcription of the coding sequence. Operably linked means that the DNA sequences being linked are typically contiguous and, where necessary to join two nucleic acids. Linking can be accomplished by ligation at convenient restriction sites or at adapters or linkers inserted in lieu thereof, or by gene synthesis, or any other method known to a person skilled in the art.

### Nucleic Acid Constructs

Gene constructs as described herein could be prepared using any cloning and/or recombinant DNA techniques, as known to a person of skill in the art, in which a nucleotide sequence encoding said insulin is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis).

### Expression Vectors

The phrase “expression vector” or “vector” generally refers to a nucleotide sequence that is capable of effecting expression of a gene or a coding sequence or of a non-coding sequence in a host compatible with such sequences. An expression vector carries a genome that is able to stabilize and remain episomal in a cell. Within the context of the invention, a cell may mean to encompass a cell used to make the construct or a cell wherein the construct will be administered. Alternatively, a vector is capable of integrating into a cell's genome, for example through homologous recombination or otherwise.
These expression vectors typically include at least suitable promoter sequences and optionally, transcription termination signals. An additional factor necessary or helpful in effecting expression can also be used as described herein.
The selection of an appropriate promoter sequence generally depends upon the host cell selected for the expression of a DNA segment. Examples of suitable promoter sequences include prokaryotic and eukaryotic promoters well known in the art (see, e.g. Sambrook and Russell, 2001, supra).

### Viral Vector

A viral vector or a viral expression vector or a viral gene therapy vector is a vector that comprises a nucleic acid construct as described herein.
A viral vector or a viral gene therapy vector is a vector that is suitable for gene therapy. Vectors that are suitable for gene therapy are described in Anderson 1998, Nature 392:25-30; Walther and Stein, 2000, Drugs 60:249-71; Kay et al., 2001, Nat. Med. 7:33-40; Russell, 2000, J. Gen. Virol. 81:2573-604; Amado and Chen, 1999, Science 285:674-6; Federico, 1999, Curr. Opin. Biotechnol. 10:448-53; Vigna and Naldini, 2000, J. Gene Med. 2:308-16; Marin et al., 1997, Mol. Med. Today 3:396-403; Peng and Russell, 1999, Curr. Opin. Biotechnol. 10:454-7; Sommerfelt, 1999, J. Gen. Virol. 80:3049-64; Reiser, 2000, Gene Ther. 7:910-3; and references cited therein. Additional references describing gene therapy vectors are Naldini 2015, Nature 5526 (7573): 351-360; Wang et al. 2019 Nat Rev Drug Discov 18 (5): 358-378; Dunbar et al. 2018 Science 359 (6372); Lukashey et al. 2016 Bioschemistry (Mosc) 81 (7): 700-708.

A particularly suitable gene therapy vector includes an adenoviral and adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications (Russell, 2000, J. Gen. Virol. 81:2573-2604; Goncalves, 2005, Virol J. 2 (1): 43) as indicated above. AAV vectors are even more preferred since they are known to result in very stable long-term expression of transgene expression (up to 9 years in dog (Niemeyer et al, Blood. 2009 Jan. 22; 113 (4): 797-806) and ˜10 years in human (Buchlis, G. et al., Blood. 2012 Mar. 29; 119 (13): 3038-41). Preferred adenoviral vectors are modified to reduce the host response as reviewed by Russell (2000, supra). Gene therapy methods using AAV vectors are described by Wang et al., 2005, J Gene Med. March 9 (Epub ahead of print), Mandel et al., 2004, Curr Opin Mol Ther. 6 (5): 482-90, and Martin et al., 2004, Eye 18 (11): 1049-55, Nathwani et al, N Engl J Med. 2011 Dec. 22; 365 (25): 2357-65, Apparailly et al, Hum Gene Ther. 2005 April; 16 (4): 426-34. Another suitable gene therapy vector includes a retroviral vector. A preferred retroviral vector for application in the present invention is a lentiviral based expression construct. Lentiviral vectors have the ability to infect and to stably integrate into the genome of dividing and non-dividing cells (Amado and Chen, 1999 Science 285:674-6). Methods for the construction and use of lentiviral based expression constructs are described in U.S. Pat. Nos. 6,165,782, 6,207,455, 6,218, 181, 6,277,633 and 6,323,031 and in Federico (1999, Curr Opin Biotechnol 10:448-53) and Vigna et al. (2000, J Gene Med 2000; 2:308-16).

Other suitable gene therapy vectors include an adenovirus vector, a herpes virus vector, a polyoma virus vector or a vaccinia virus vector.

### Adeno-Associated Virus Vector (AAV Vector)

The terms “adeno associated virus”, “AAV virus”, “AAV virion”, “AAV viral particle” and “AAV particle”, used as synonyms herein, refer to a viral particle composed of at least one capsid protein of AAV (preferably composed of all capsid protein of a particular AAV serotype) and an encapsulated polynucleotide of the AAV genome. If the particle comprises a heterologous polynucleotide (i.e. a polynucleotide different from a wild-type AAV genome, such as a transgene to be delivered to a mammalian cell) flanked by AAV inverted terminal repeats, then they are typically known as a “AAV vector particle” or “AAV viral vector” or “AAV vector”. AAV refers to a virus that belongs to the genus Dependovirus family Parvoviridae. The AAV genome is approximately 4.7 Kb in length and it consists of single strand deoxyribonucleic acid (ssDNA) that can be positive or negative detected. The invention also encompasses the use of double stranded AAV also called dsAAV or scAAV. The genome includes inverted terminal repeats (ITR) at both ends of the DNA strand, and two open reading frames (ORFs): rep and cap. The frame rep is made of four overlapping genes that encode proteins Rep necessary for the AAV lifecycle. The frame cap contains nucleotide sequences overlapping with capsid proteins: VP1, VP2 and VP3, which interact to form a capsid of icosahedral symmetry (see Carter and Samulski, Int J Mol Med 2000, 6 (1): 17-27, and Gao et al, 2004). A preferred viral vector or a preferred gene therapy vector is an AAV vector. An AAV vector as used herein preferably comprises a recombinant AAV vector (rAAV vector). A “rAAV vector” as used herein refers to a recombinant vector comprising part of an AAV genome encapsidated in a protein shell of capsid protein derived from an AAV serotype as explained herein. Part of an AAV genome may contain the inverted terminal repeats (ITR) derived from an adeno-associated virus serotype, such as AAV1, AAV2, AAV3, AAV4, AAV5 and others. Preferred ITRs are those of AAV2.
Protein shell comprised of capsid protein may be derived from any AAV serotype. A protein shell may also be named a capsid protein shell. rAAV vector may have one or preferably all wild type AAV genes deleted, but may still comprise functional ITR nucleotide sequences. In this context, functionality refers to the ability to direct packaging of the genome into the capsid shell and then allow for expression in the host cell to be infected or target cell. In the context of the present invention a capsid protein shell may be of a different serotype than the rAAV vector genome ITR.
A nucleic acid molecule represented by a nucleotide sequence of choice, encoding an oligonucleotide of the invention, is preferably inserted between the rAAV genome or ITR sequences as identified above, for example an expression construct comprising an expression regulatory element operably linked to a coding sequence and a 3′ termination sequence.
“AAV helper functions” generally refers to the corresponding AAV functions required for rAAV replication and packaging supplied to the rAAV vector in trans. AAV helper functions complement the AAV functions which are missing in the rAAV vector, but they lack AAV ITRs (which are provided by the rAAV vector genome). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73 (2): 1309-1319) or U.S. Pat. No. 5,139,941, incorporated herein by reference. The AAV helper functions can be supplied on an AAV helper construct. Introduction of the helper construct into the host cell can occur e.g. by transformation, transfection, or transduction prior to or concurrently with the introduction of the rAAV genome present in the rAAV vector as identified herein. The AAV helper constructs of the invention may thus be chosen such that they produce the desired combination of serotypes for the rAAV vector's capsid protein shell on the one hand and for the rAAV genome present in said rAAV vector replication and packaging on the other hand.
“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via plasmids, as described in U.S. Pat. No. 6,531,456 incorporated herein by reference.
“Transduction” refers to the delivery of an insulin into a recipient host cell by a viral vector. For example, transduction of a target cell by a rAAV vector of the invention leads to transfer of the rAAV genome contained in that vector into the transduced cell. “Host cell” or “target cell” refers to the cell into which the DNA delivery takes place, such as the muscle cells of a subject. AAV vectors are able to transduce both dividing and non-dividing cells.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that an oligonucleotide, a viral vector or a composition as defined herein may comprise additional component(s) than the ones specifically identified, said additional component(s) not altering the unique characteristic of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated. All patent and literature references cited in the present specification are hereby incorporated by reference in their entirety.

When a structural formula or chemical name is understood by the skilled person to have chiral centers, yet no chirality is indicated, for each chiral center individual reference is made to all three of either the racemic mixture, the pure R enantiomer, and the pure S enantiomer. The word “about” or “approximately” when used in association with a numerical value (e.g. about 10) preferably means that the value may be the given value (of 10) more or less 0.1% of the value.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

The following examples are offered for illustrative purposes only, and are not intended to limit the scope of the present invention in any way.

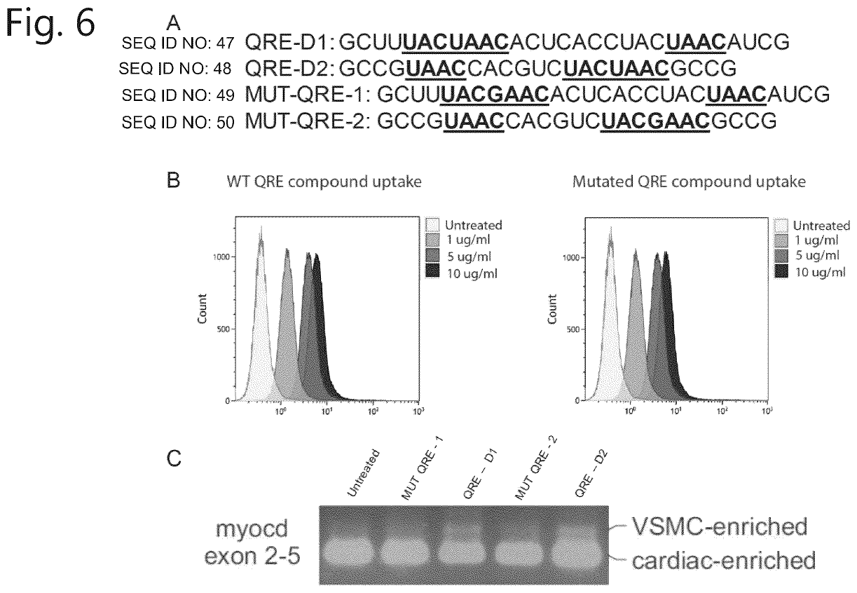

TABLE 1
Sequences of the sequence listing

| | DNA/RNA/Amino | |
|---|---|---|
| SEQ ID NO | acid (aa) | Name |
| | RNA | Mutated core QKI binding site |
| | RNA | Core QKI binding site |
| | RNA | Core QKI binding site (generic) |
| | RNA | Half QKI binding site (generic) |
| | RNA | Half QKI binding site |
| | RNA | Core QKI binding site |
| | RNA | Half QKI binding site |
| | RNA | Half QKI binding site |
| | RNA | Half QKI binding site |
| 10 | RNA | Oligonucleotide (1) |
| 11 | Modified RNA | Oligonucleotide (1) |
| 12 | RNA | Oligonucleotide (2) |
| 13 | Modified RNA | Oligonucleotide (2) |
| 14 | RNA | Oligonucleotide (3) |
| 15 | Modified RNA | Oligonucleotide (3) |
| 16 | aa | QKI-5 |
| 17 | DNA | QKI-5 |
| 18 | aa | QKI-6 |
| 19 | DNA | QKI-6 |
| 20 | aa | QKI-7 |
| 21 | DNA | QKI-7 |
| 22-25 | Modified RNA | Oligonucleotides of table 2 |
| 26-45, 83-86 | DNA | Primers of table 3 |
| | RNA | Mutated motif |
| 47-50 | Modified RNA | Oligonucleotides of FIG. 6 |
| | RNA | 2 core QKI binding sites |
| | RNA | 2 core QKI binding sites |
| | RNA | 2 core QKI binding sites |
| 54-61, 70-73, | RNA | oligonucleotides |
| 94-110 | | |
| 62-65, 66-69, | Modified RNA | oligonucleotides |
| 74-82, 90-93, | | |
| 111-118 | | |
| | RNA | QKI binding site |
| 119 | Modified RNA | Control oligonucleotide scr |

### EXAMPLES

### Material and Methods

### Animals

All animal experiments were approved by the Institutional Animal Care and Use Committee of the Leiden University Medical Center.

### Bronchopulmonary Dysplasia (BPD) Rat Model

Adult Wistar rats (6 months old; N=6) were exsanguinated after induction of anesthesia with an intraperitoneal injection of ketamine (50 mg/kg) and xylazine (50 mg/kg). Organs were stored at −80° C. until isolation of RNA for real time RT-PCR.
For each intervention experiment, newborn Wistar rat pups from 3-5 litters were pooled and assigned ad random to 4 experimental groups: an oxygen-RNA-Cont group (N=6), an oxygen-RNA-QRE group (N=6) and two room air (RA)-exposed control groups (N=6 each). All oxygen-exposed pups were housed together in Plexiglas chambers. Pups were fed by foster dams and received a single subcutaneous injection of RNA-Cont or RNA-QRE (Thermo Scientific, St. Leon-Rot, Germany) at a concentration of 40 mg/kg dissolved in 100 μl 0.9% NaCl on day 2 after birth. To avoid oxygen toxicity, foster dams were rotated daily: 24 hours in hyperoxia and 48 hours in RA. Oxygen concentration, body weight, evidence of disease, and mortality were recorded daily. Lung and heart tissue was collected on days 10 (for histology and morphometry only). Separate experiments were performed to obtain: [1] formalin fixed lung and heart tissue for histology (N=8); and [2] lung homogenates for fibrin deposition (N=8). For all parameters, at least two independent experiments were performed.

### UUO Mouse Model

8-week-old C57Bl6 wild-type mice (Jackson Laboratories, Bar Harbor, ME) were used. UUO was performed through a left flank incision under general anesthesia. The ureter was identified and ligated twice at the level of the lower pole of the kidney with 2 separate silk ties. Either SEQ ID NO: 64 or SEQ ID NO: 65 was administered intravenously at a concentration of 40 mg/kg 24 hours prior to surgery or 2 days post-injury. The mice were subsequently fed a chow diet until sacrifice at either 5 or 10 days post-surgery.

### Histology

### Ischemic Reperfusion Injury (IRI) and Unilateral Ureter Obstruction (UUO)

Formalin-fixed, paraffin-embedded IRI mouse kidney sections (4 μm) were deparaffinized, after which either heat-induced antigen retrieval (QKI-5 and QKI-7) or Nat-citrate antigen retrieval (QKI-6 and pan-QKI) were applied. The samples were peroxidase and mouse IgG blocked (Mouse-on-Mouse detection kit; Vector Laboratories, Burlingame, CA, USA), after which the tissue sections were immunostained using: QKI-5 (clone N195A/16 at 1:100 dilution; UC Davis/Neuromab, CA, USA), QKI-6 (clone N182/17 at 1:100 dilution; UC Davis/Neuromab, CA, USA), QKI-7 (clone N183/15 at 1:100 dilution; UC Davis/Neuromab, CA, USA) and pan-QKI (clone N147/6 at 1:100 dilution; UC Davis/Neuromab). IgG1 served as isotype control for QKI-5/6/7, while IgG2b was used as isotype control for pan-QKI stainings. Following washing, goat anti-mouse HRP secondary antibody (DAKO, K3468; Glostrup, Denmark) was applied. The sections were stained with DAB and counterstained using Mayer's hematoxylin.
For UUO immunohistochemistry of unilateral ureter obstruction mouse kidney material, 4 μm fixed-frozen cryosections were obtained after freezing tissue samples embedded in O.C.T. (TissueTek, Torrance, CA, USA) after which the mould was placed an isopentane solution cooled with liquid nitrogen. The kidney sections were subsequently air-dried, fixed at room temperature in acetone, air-dried and stored at −20° C. Prior to primary antibody staining, the sections were washed in PBS at room temperature and incubated in blocking buffer (PBS with +1% BSA and 1% FCS) for 1 hour. Slides were then incubated with either QKI-5 (clone N195A/16 at 1:100 dilution; UC Davis/Neuromab, CA, USA), QKI-6 (clone N182/17 at 1:100 dilution; UC Davis/Neuromab, CA, USA), QKI-7 (clone N183/15 at 1:100 dilution; UC Davis/Neuromab, CA, USA) and pan-QKI (clone N147/6 at 1:100 dilution; UC Davis/Neuromab). IgG1 served as isotype control for QKI-5/6/7, while IgG2b was used as isotype control for pan-QKI stainings. For DAB staining, following washing, goat anti-mouse HRP secondary antibody (DAKO, K3468; Glostrup, Denmark) was applied, after which DAB was applied and the sections counterstained using Mayer's hematoxylin. For fluorescence microscopy, primary antibodies were incubated for 3 h at room temperature or at 4° C. overnight, after which extensive washing was performed prior to incubation with the appropriate Alexa® labelled secondary antibody (Invitrogen, Carlsbad, CA, USA) was applied in blocking buffer. Subsequently, slides were embedded in Prolong Gold (Invitrogen) containing DAPI for nuclear staining. Imaging was performed on a Leica DM5500 or Andor Dragonfly spinning disk microscopy (Oxford Instruments, Abingdon, UK) and data analyzed using Imaris software package (Oxford Instruments).
Histochemical analysis for collagen content was performed using picrosirius red staining on formalin-fixed, paraffin-embedded tissues. CLK and UUO kidneys were fixed in 3.7% formalin in PBS for 2 h, after which they were placed in 70% ethanol overnight followed by paraffin-embedding according to standard protocols. For analyses, 4 μm sections were prepared. Prior to staining, slides were first deparaffinized in Xylene, then taken through a series of solutions decreasing in ethanol percentage, and finally placed in ddH₂O for 30 min. Slides were submerged in sirius red F3B solution (0.1% Direct Red 80, Sigma Aldrich) in a saturated aqueous solution of picric acid for 1 h at room temperature. Slides were washed in 3 stages of acidified water consisting of 0.005% glacial acetic acid (Millipore-Sigma, Carlsbad, CA, USA). Subsequently, slides were dehydrated in sequential fashion in 100% ethanol and Xylene and embedded in Entellan. Images were taken using a Leica DMI4400B microscope and collagen deposition was quantified using ImageJ (Schneider, C. A. et al. 2020. Nature Methods, 9, 671-675).

### Bronchopulmonary Perfusion Model

Formalin-fixed, paraffin-embedded, 4 μm-thick heart and lung sections were deparaffinized and subsequently stained with hematoxylin and eosin (HE). In addition, lung tissue sections were immune-stained with anti-ED-1 (monocytes and macrophages; 1:5), anti-myeloperoxidase (MPO, RB-373-A1, Thermo Fisher Scientific, Fremont, CA, USA; diluted 1:1,500), anti-α smooth muscle actin (ASMA, A2547, Sigma-Aldrich, St. Louis, MO, USA; diluted 1:20,000), anti-von Willebrand factor (vWF, A0082, Dako Cytomation, Glostrup, Denmark; diluted 1:4,000), anti-collagen III (COL3A1, ab7778; Abcam: diluted 1:3000), using the chromogenic substrate NovaRed or NovaRed and Vector SG Substrate on ASMA and vWF double stained sections, respectively, as recommended by the manufacturer (Vector, Burlingame, CA, USA), and counterstained briefly with hematoxylin using standard methods (de Visser, Y. P. et al., 2010, American Journal of Respiratory Critical Care, 182 (10): 1239-1250; Wagenaar, G. T. M. et al., 2004, Free Radical Biological Medicine, 36 (6): 782-801). Furthermore, elastin was visualized on Hart's stained lung sections (Simon, D. M. et al., 2010, Respiratory Research, 11:1-9). For morphometry of the lung, an eye piece reticle with a coherent system of 21 lines and 42 points (Weibel type II ocular micrometer; Olympus, Zoeterwoude, The Netherlands) was used (Wagenaar, G. T. M. et al., 2004, Free Radical Biological Medicine, 36 (6): 782-801). We used different (immuno) histochemically stained lung sections for each quantification, except for alveolar crest and pulmonary arteriolar wall thickness, which were determined on the same ASMA stained section. To investigate alveolar enlargement in experimental BPD, we studied the number of alveolar crests to exclude potential effects of heterogeneous alveolar development. The number of alveolar crests (Yi, M. et al., 2004, American Journal of Respiratory Critical Care, 170 (11): 1188-1196), determined on lung sections stained immunohistochemically for ASMA, were assessed in 10 non-overlapping fields at a 400× magnification for each animal and were normalized to tissue and field. The density of ED-1 positive monocytes and macrophages or MPO-positive neutrophilic granulocytes was determined in the alveolar compartment by counting the number of cells per field. Results were expressed as cells per mm². Per experimental animal 20 fields in one section were studied at a 400× magnification. Pulmonary alveolar septal thickness was assessed in HE-stained lung sections at a 400× magnification by averaging 100 measurements per 10 representative fields. Capillary density was assessed in lung sections stained for vWF at a 200× magnification by counting the number of vessels per field. At least 10 representative fields per experimental animal were investigated. Results were expressed as relative number of vessels per mm². Pulmonary arteriolar wall thickness was assessed twice in lung sections stained for elastin or ASMA at a 1000× magnification by averaging at least 10 vessels with a diameter of less than 30 μm per animal for each of the two different staining methods. Medial wall thickness was calculated from the formula

$$“ percent wall thickness ” = \frac{2 * wall \cdot thickness}{external \cdot diameter} * 100 ”$$

(Koppel, R. et al., 1994, Pediatric Research, 36 (6): 763-770). Muscularization of small arterioles (<50 μm) was determined on ASMA and vWF double stained lung sections, using the 50% ASMA layer circumference as a cutoff at a 400× magnification by counting 50 blood vessels per lung section (Dunnhill, M. S., 1962, Thorax, 17 (4): 320-328). Fields containing large blood vessels or bronchioli were excluded from the analysis. Thickness of the right and left ventricular free walls was assessed in a transversal HE-stained section taken halfway the long axis at a 40× magnification by averaging 6 measurements per structure. RVH was calculated for each heart by dividing average RV free wall thickness and average LV free wall thickness. For morphometric studies in lung and heart, 6-8 and 10 rat pups per experimental group were studied, respectively. Quantitative morphometry was performed by two independent researchers blinded to the treatment strategy using the NIH Image J program (de Visser, Y. P. et al., 2010, American Journal of Respiratory Critical Care, 182 (10): 1239-1250; de Visser, Y. P. et al., 2012, American Journal of Physiology Lung Cellular Molecular Physiology, 302 (1): L56-L57; Yi, M. et al., 2004, American Journal of Respiratory Critical Care, 170 (11): 1188-1196.

### Oligonucleotide-Based Decoy Design

Oligonucleotides (decoys) were designed to mimic the Quaking response element. Ribonucleic acids in the decoys are generally fully phosphorothioated, except for in vivo decoys which contained phosphorothioate modifications at the 5′ (2 most 5′ nucleotides and 4 most 3′ nucleotides for in vivo BPD and UUO studies (as depicted below). All nucleotides contain 2′-O-methyl sugar moieties. The decoys are 27 nucleotides in length or range in length from 12 to 36 nucleotides in length. Decoys were constructed containing 5′ Dy647 phosphoramidite (excitation peak at 652 and emission peak at 673) for in vivo detection and 3′ cholesterol tag for improvement of cellular uptake. This was done for BPD rat studies (see below) and in unconjugated form for UUO mouse studies. Biotin-labelled decoys were constructed to determine binding affinity for QKI protein. Secondary structure and binding energy of the oligonucleotide-based decoys were predicted using RNA structure.

### Cellular Transfection

Following seeding at 2.0×10⁵ cells/cm² one day prior to transfection, roughly 80% confluent HEK293 or U87MG cells were transfected according to manufacturer's instructions (Mirus, Madison, WI, USA). In brief, the transfection reagent was warmed to room temperature and vortexed briefly. An appropriate amount of Optimem culture medium (serum-free) was placed in a sterile tube and a defined concentration of dcRNA added to the tube. The dcRNA solution was mixed gently by pipetting and 7.5 μL TransIT-LT1 solution added to the sample. The sample was gently mixed and incubated at room temperature for 30 minutes. Subsequently, the mixture was added dropwise to the cells for 24 h after which the cells were harvested in Trizol to harvest RNA to allow for assessment of splicing events.

TABLE 2
sequence of oligonucleotides

| Decoy | |
|---|---|
| name | Sequence (5′ to 3′ orientation) |
| RNA- | Dy647-oG*oC*oUoUoUoAoCoGoAoAoCoAoCoAoaGoU |
| Cont-1 | oAaCoGoAoAoCoA*oU*oC*oG*-chol |
| SEQ ID | |
| NO: 22 | |
| RNA- | oG*oC*oUoUoUoAoCoGoAoAoCoAdoCoAoaGoUoAoCo |
| Cont-2 | GoAoAoCoA*oU*oC*oG*-chol |
| SEQ ID | |
| NO: 23 | |
| RNA- | Dy647-oG*oC*oUoUoUoAoCoUoAoAoCoAoCoAoGoUo |
| QRE-1 | AoCoUoAoAoCoA*oU*oC*oG*-chol |
| SEQ ID | |
| NO: 24 | |
| RNA- | oG*oC*oUoUoUoAoCoUoAoAoCoAoCoAoGoUoAoCoUo |
| QRE-2 | AoAoCoA*oU*oC*oG*-chol |
| SEQ ID | |
| NO: 25 | |

*Indicates a phosphorothioate linkage;
o indicates 2′-O-methyl ribose;
Underlined sequence indicates core region(s) of QRE;
bold indicates nucleotide change relative to QRE

Real-Time qPCR

TK173 cells were lysed in Trizol and RNA was isolated using the RNeasy kit (Qiagen). A DNAse I (Qiagen) treatment was added to remove excess DNA during the isolation and cDNA was synthesized using Promega reverse transcriptase, DTT, dNTPs and random primers. Real time PCR was performed on a CFX384 Touch™ Real-Time PCR Detection System (Bio Rad) with SYBR™ Select Master Mix (Thermo Fisher) and the following primers:

TABLE 3
primers used (SEQ ID NO: 26-45, 83-86)

| Gene | Forward primer | Reverse primer |
|---|---|---|
| GAPDH | TTCCAGGAGCGAGAT | CACCCATGACGAACA |
| (human) | CCCT (26) | TGGG (28) |
| GAPDH | ACTCCCACTCTTCCA | CACCACCCTGTTGCT |
| (mouse) | CCTTC (27) | GTAG (29) |
| QKI-5 | CTGTCATGCCAAACG | GATGGACACGCATAT |
| (human) | GAAC (30) | CGTG (32) |
| QKI-5 | CCCAGTGGTGTGTTA | CCTTTGGTAAGGATG |
| (mouse) | GGTGC (31) | GACACG (33) |
| QKI-6 | CTGTCATGCCAAACG | CGTTGGGAAAGCCAT |
| (human) | GAAC (34) | AC (36) |
| QKI-6 | AAGTTACTGGTACCT | GAAAGCCATACCCTA |
| (mouse) | GCGGCTGAA (35) | ACACCACTG (37) |
| QKI-7 | CTGTCATGCCAAACG | GACTGGCATTTCAAT |
| (human) | GAAC (38) | CCAC(40) |
| QKI-7 | AAGTTACTGGTACCT | GCATGACTGGCATTT |
| (mouse) | GCGGCTGAA (39) | CAATCCACTCT (41) |
| ACTA2 | GTGCTGGACTCTGGA | AATAGCCACGCTCAG |
| (human) | GATGG (42) | TCAGG (44) |
| ACTA2 | CGTGGCTATTCCTTC | GCAGTTCGTAGCTCT |
| (mouse) | GTGAC (43) | TCTCC (45) |
| ADD3 | ACCAGCTCCTCCTAA | TCACTCGCTTAGCAA |
| (human) | CCCAT (83) | GCTCAT (84) |
| ACTB | TGCGTGACATTAAGG | TGAAGGTAGTTTCGT |
| (human) | AGAAG (85) | GGATG (86) |

Note:
(h) denotes human primer sequences;
(m) denotes mouse primer sequences

TABLE 4
Additional sequences of oligonucleotides

| SEQ | |
|---|---|
| ID | |
| NO | Sequence |
| 54 | GCUUUACGAACACAGUACGAACAUCG |
| 55 | GCUUUACUAACACAGUACUAACAUCG |
| 56 | GCUUUACGAACACUCACCUACUAACAUCG |
| 57 | GCUUUACUAACACUCACCUACUAACAUCG |
| 58 | GCCGUAACCACGUCUACGAACGCCG |
| 59 | GCCGUAACCACGUCUACUAACGCCG |
| 60 | GCUUUACGAACACAGAUACGAACAUCG |
| 61 | GCUUUACUAACACAGAUACUAACAUCG |
| 62 | Bio-oGoCoUoUoUoAoCoGoAoAoCoAoCoAoGoAoUoAoCoGoAoAoCoAoUoCoG |
| 63 | Bio-oGoCoUoUoUoAoCoUoAoAoCoAoCoAoGoAoUoAoCaUoAoAoCoAoUoCoG |
| 64 | oG*oC*OU*OU*OU*oA*oC*OG*oA*oA*oC*oA*oC*oA*G*oA*OU*A*oC*G*A*A*oC*A*OU*oC*OG |
| 65 | oG*oC*OU*OU*OU*oA*oC*OU*oA*oA*oC*A*oC*A*OG*A*OU*A*C*OU*A*A*C*A*U*C*OG |
| 66 | Bio-oAoCoUoAoAoCdTdTdToAoCoUoAoAoCdTdTdToAoCoUoAoAoCdTdTdToAoCoUoAoAoC |
| 67 | Bio-oAoCoUoAoAoCnTnTnToAoCoUoAoAoCnTnTnToAoCoUoAoAoCnTnTnToAoCoUoAoAoC |
| 68 | Bio-oAoCoUoAoAoCoUoUoUoAoCoUoAoAoCoUoUoUoAoCoUoAoAoCoUoUoUoAoCoUoAoAoC |
| 69 | Bio-oAoCoUoAoAoC[C9]oAoCoUaAoAoC[C9]oAoCoUoAoAoC[C9]oAoCoUoAoAoC |
| 70 | ACUAACACUAAC |
| 71 | ACUAACACUAACACUAAC |
| 72 | ACUAACACUAACACUAACACUAAC |
| 73 | ACUAACACUAACACUAACACUAACACUAACACUAAC |
| 74 | Bio-oAoCoUoAoAoCoAoCoUoAoAoC |
| 75 | Bio-oAoCoUoAoAoCoAoCoUoAoAoCoAoCoUoAoAoC |
| 76 | Bio-oAoCoUoAoAoCoAoCoUoAoAoCoAoCoUoAoAoCoAoCoUoAoAoC |
| 77 | Bio-oAoCoUoAoAoCoAoCoUoAoAoCoAoCoUoAoAoCoAoCoUoAoAoCoAoCoUoAoAoCoAoCoUoAoAoC |
| 78 | Bio-ACUAACACUAACACUAACACUAAC |
| 79 | Bio-A*C*U*A*A*C*A*C*U*A*A*C*A*C*U*A*A*C*A*C*U*A*A*C |
| 80 | oAoCoUoAoAoCoAoCoUoAoAcCaAoCoUoAoAoC |
| 81 | oAoCoUoAoAoCoAoCoUoAoAoCoAoCoUoAoAoCAoCoUoAoAoC |
| 82 | oA*oC*oU*oA*oA*oC*oA*oC*oU*oA*oA*oC*oA*oC*oU*oA*oA*oC*oA*oC*oU*oA*oA*oC |

Note:
Dy647 denotes a fluorescent molecule;
Chol denotes cholesterol moiety;
Bio denotes C6 biotin;
oA, oC, oG, OU denotes 2′-O-Methyl modified ribonucleic acids;
*denotes phosphorothioate modification;
dA, dC, dG, dT denotes unmodified deoxyribonucleic acids;
nA, nC, nG and nU denotes locked nucleic acid residues;
[C9] denotes 9-triethylene glycol (TEG) spacer

### Splicing Assay

HEK293 cells were grown in DMEM supplemented with 8% (v/v) FCS, penicillin/streptomycin and oligonucleotides were transfected using lipofectamine 2000. RNA was extracted using TRIzol reagent (Invitrogen) and RNA was reverse transcribed using M-MVL Reverse Transcriptase (Promega) and PCR amplified, applying specific primers, using GoTaq G2 DNA Polymerase (Promega). PCR products were analysed on 2% agarose gels.

### Pull Down Assay

Cellular extracts were generated from HEK 293 cells using the NE-PER Nuclear and Cytoplasmic Extraction reagents (Thermo Scientific). Prior usage cellular extracts were treated with Complete® protease inhibitor (Roche) and RNasin RNase inhibitor (Promega). Strepavidin beads (Cytiva) were washed in 2× binding buffer (10 mM Tris-HCl (pH 7.5), 1 mM EDTA, 2M NaCl) and after addition of the biotinylated oligonucleotide the solution was incubated for 30 min at RT using gentle rotation. After two washing steps with binding buffer, beads were resuspended in 1× washing buffer and following the addition of the cellular extract incubated for 3 h at 4° C. using gentle rotation. Samples were washed 3× with protein binding buffer (20 mM HEPES (pH 7.5), 50 mM KCl, 10% glycerol, 5 mM MgCl₂), supplemented with 10 mM DTT (10×) and Complete® protease inhibitor (Roche) and RNasin RNase inhibitor (Promega) prior usage. The pulled down fraction was lysed in 4×LDS sample buffer (Thermo Scientific) supplemented with 1M DTT (10×) and analysed by Western Blot analysis.

### Western Blot Analysis

Various human and mouse kidney cell lines were cultured and harvested in RIPA buffer (Sigma Aldrich, St. Louis, MO, USA) containing Complete® protease inhibitors (Roche, Basel, Switzerland), followed by BCA-based protein quantitation (Thermo Fisher Scientific, Waltham, MA, USA) to ensure equivalent protein loading. Protein lysates were resolved by polyacrylamide gel electrophoresis (Bio-Rad, Hercules, CA, USA). Subsequently, gels were transferred to nitrocellulose membranes (Bio-Rad) using the Trans-Blot Turbo® (Bio-Rad). Membranes were blocked overnight at 4° C. in 5% skim milk powder (Nutricia, Zoetermeer, the Netherlands) in PBS with 0.1% tween-80 (PBST), after which primary antibodies were incubated for 2 h at room temperature or overnight at 4° C. Primary antibodies utilized were QKI-5 (clone N195A/16 at 1:2000 dilution; UC Davis/Neuromab, CA, USA), QKI-6 (clone N182/17 at 1:2000 dilution; UC Davis/Neuromab, CA, USA), QKI-7 (clone N183/15 at 1:1000 dilution; UC Davis/Neuromab, CA, USA) and beta-actin was employed as a loading reference (Abcam, Cambridge, UK). The appropriate HRP-labelled secondary antibodies (Dako, Glostrup, Denmark) were incubated for 1 hour at room temperature, followed by extensive washing with PBST. Bands were visualized using SuperSignal West Dura® Extended Duration Substrate (Thermo Fisher Scientific).

### Results

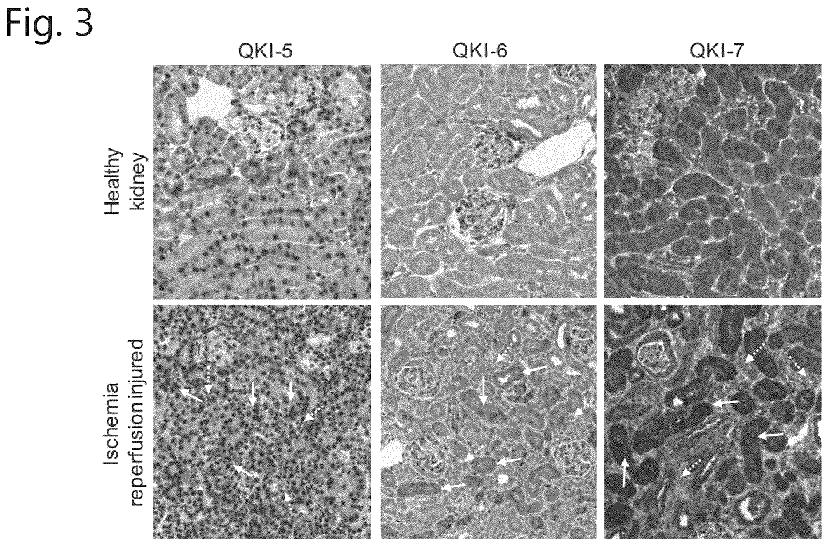

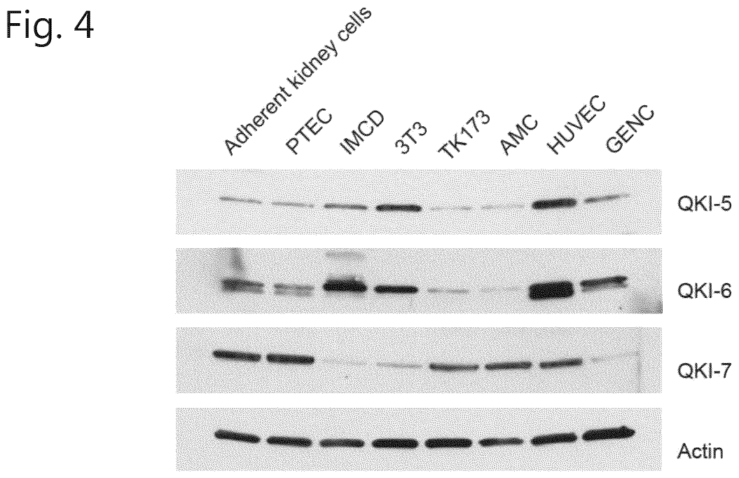

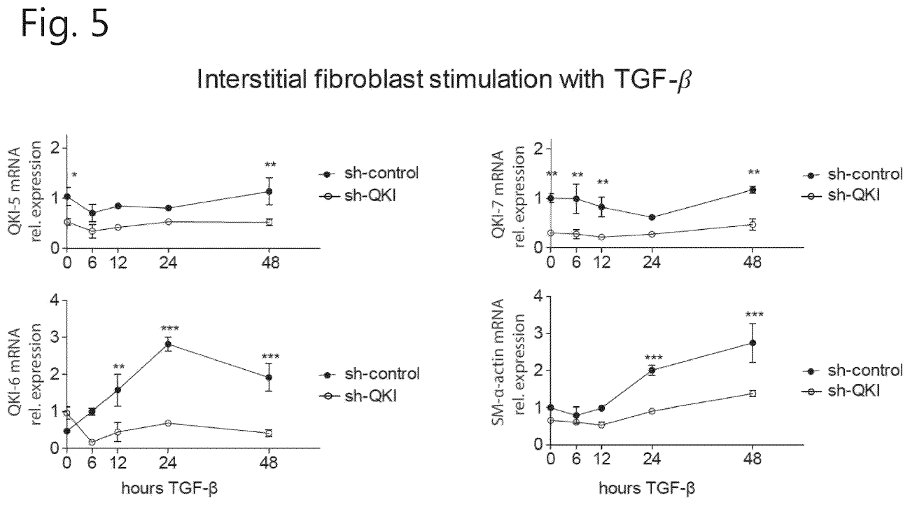

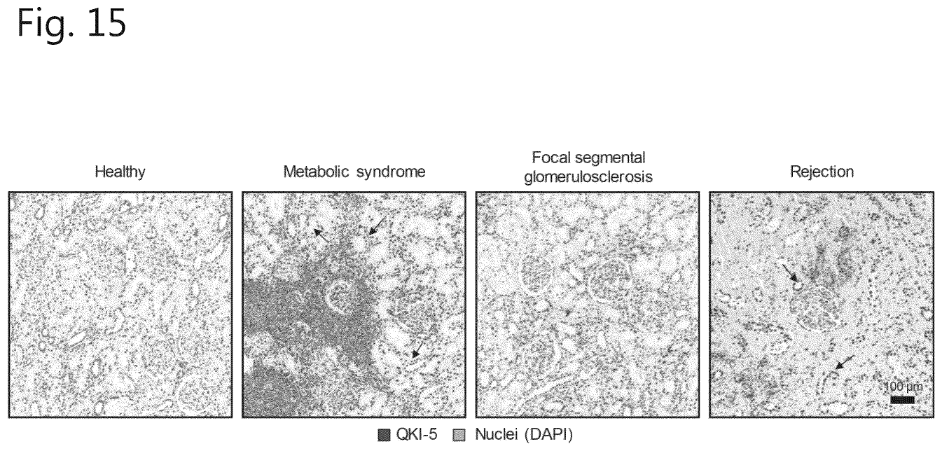

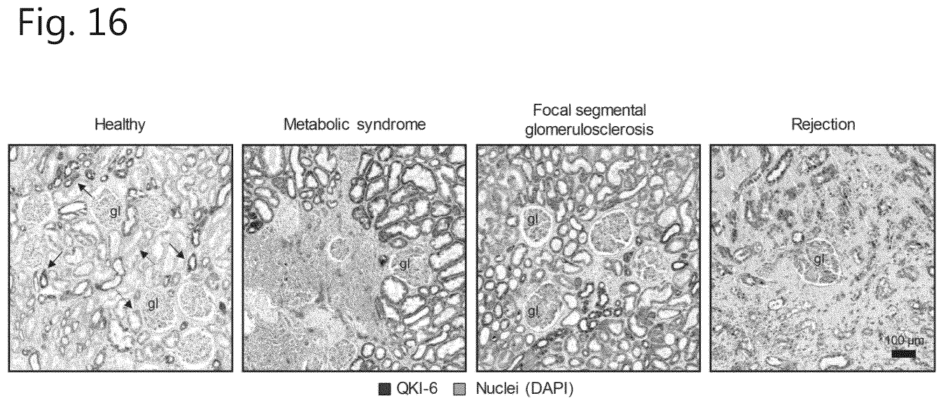

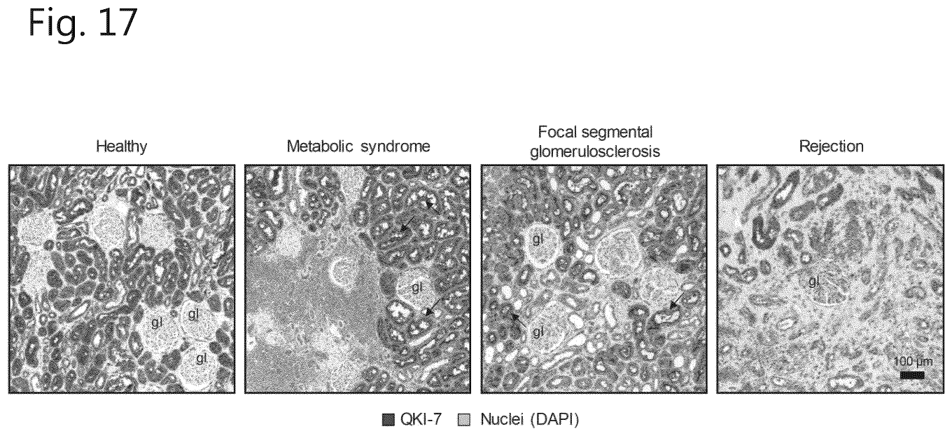

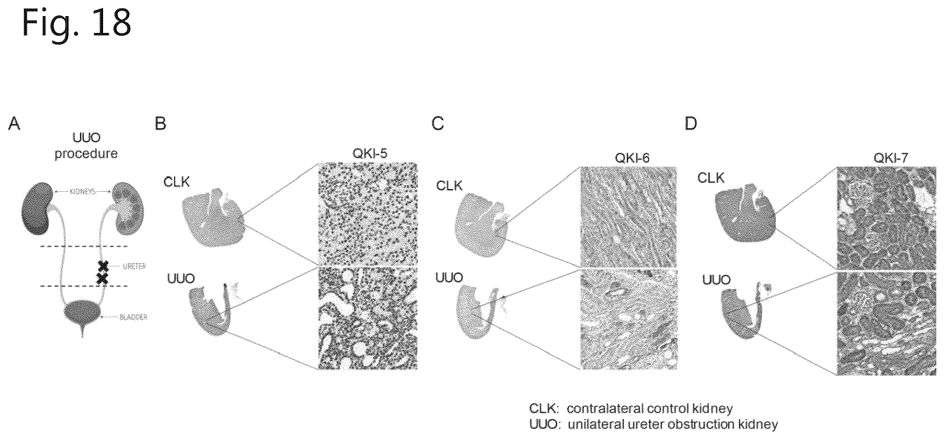

Kidney Injury is Associated with Augmented QKI Expression
Increases in QKI expression have previously been found to impact inflammatory and fibrotic responses to cellular and tissue injury (van der Veer, E. P. et al., 2013, Circulation Research, 113 (9): 1065-1075; de Bruin, R. G. et al., 2016, Nature Communications, 7:10846; de Bruin, R. G. et al., 2020, Epigenomics, 4 (2)). To assess the role of QKI in the context of kidney injury, we obtained pathologic human kidney material and performed immunohistochemical analysis for expression levels of the distinct QKI isoforms, namely QKI-5 (FIG. 15), QKI-6 (FIG. 16) and QKI-7 (FIG. 17). These studies revealed a particularly strong role for increased QKI-6 expression in metabolic syndrome, focal segmental glomerulosclerosis and (acute) rejection as compared to healthy kidney (FIG. 16, panels 2-4 and panel 1, respectively).
Having identified that QKI protein expression is augmented in the setting of human kidney injury, we also scored QKI expression in experimental animal models of kidney injury. First, we assessed QKI expression following ischemia reperfusion injury (IRI) of C57BL6 mice, an injury process that is associated with a robust inflammatory and oxidative stress response to hypoxia and tissue reperfusion, which disturbs organ function. In the setting of the kidney, evidence suggests that the endothelial and epithelial cells are most affected by this mode of injury (Chatauret, N. et al. 2014, Progress in Urology, 24: S4-12), where tissue damage leads to mononuclear cell recruitment and infiltration in combination with cytokine release that triggers acute tubular necrosis and local production of matrix, as observed in acute kidney injury (FIG. 3, bottom left panels). IRI was associated with marked increases in QKI-5, QKI-6 and QKI-7 protein expression (FIG. 3), with in particular striking increases in QKI-6 observed in the proximal tubules of the mouse kidney. Also notable was the apparent shift in QKI-7 expression upon kidney injury from almost exclusively cytoplasmic to a more diffuse expression in both the nucleus and cytoplasm.
Next, we complemented these studies with examination of QKI expression levels in unilateral ureter obstructed (UUO) mouse kidneys (FIG. 18A). As shown in FIG. 18B, UUO resulted in a marked accumulation of QKI-5⁺ nuclei in glomeruli and kidney interstitium (bottom panel), which could be the result of mesangial expansion and leukocyte infiltration/cellular proliferation, respectively. Furthermore, QKI-6 and QKI-7 expression displayed regional enhancement in expression, in particular in tubules and the glomerular parietal epithelium (FIG. 18 C-D bottom panels).
IRI was associated with marked increases in QKI-5, QKI-6 and QKI-7 protein expression (FIG. 3), with in particular striking increases in QKI-6 observed in the proximal tubules of the mouse kidney. Also notable was the apparent shift in QKI-7 expression upon kidney injury from almost exclusively cytoplasmic to a more diffuse expression in both the nucleus and cytoplasm. Importantly, injury to proximal tubular epithelial cell (PTEC) is well established to play a critical role in driving the shift from acute kidney injury (AKI) to chronic kidney disease (CKD), as upon injury these cells actively secrete transforming growth factor-β (TGF-β) into the local surroundings. Unabided TGF-β production is detrimental in that it: 1) perpetuates tubular injury; 2) promotes monocyte infiltration and macrophage chemotaxis; 3) stimulates endothelial distress; and 4) activates interstitial fibroblasts to synthesize extracellular matrix (Gewin, L. S., 2019, Nephron, 143:154-157). As shown in FIG. 4, the expression levels of the distinct QKI isoforms varies among various kidney-resident cell-types, with QKI expression levels being relatively low in quiescent interstitial fibroblasts. To discern whether activation of interstitial fibroblasts by TGF-β impacts QKI expression levels, we treated a human interstitial fibroblastic cell line (TK173 cells) with TGF-β for 48 hours and subsequently harvested RNA (FIG. 5). These studies revealed increased expression of QKI-5, -6 and -7 mRNAs upon stimulation with TGF-β, with in particular a striking increase in QKI-6 expression at 24 hours post-treatment. Also notable was the fact that lentiviral-mediated abrogation of QKI expression (shRNAs against all QKI isoforms) resulted in markedly decreased ASMA mRNA levels in TK173 cells in response to TGF-β stimulation, suggesting that a reduction in QKI expression prevents interstitial fibroblasts from adopting the myofibroblast, or pro-fibrotic, phenotype. Collectively, these studies suggest that the application of methods to attenuate QKI expression or activity could be beneficial in preventing kidney injury-induced inflammation and fibrosis.

### Design of a QKI Inhibiting RNA-Based Decoy

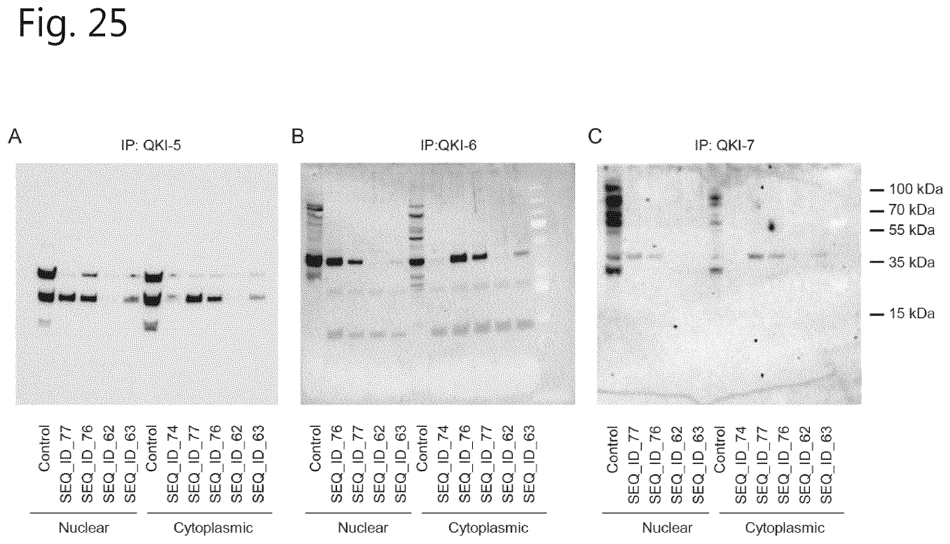

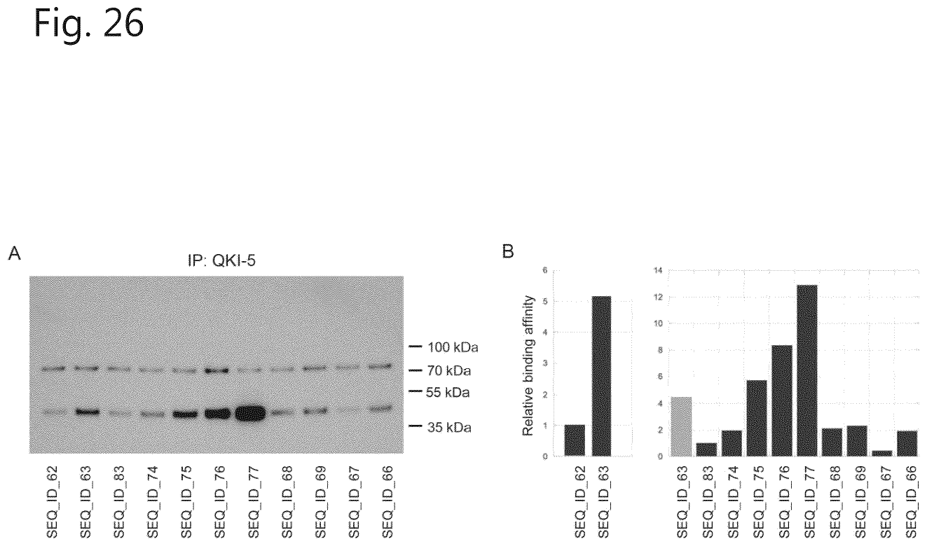

Since QKI expression is clearly upregulated in the setting of acute kidney injury (FIGS. 3 and 15-18), we were prompted to determine whether inhibition of QKI activity could impact splicing in a cell-based model. For this, we first designed two oligonucleotides that varied in length and consisted either of dual QKI core sites or consisted of a core and a half site (QRE-D1 and QRE-D2, SEQ ID NO: 47 and SEQ ID NO: 48). The spacing in between the core-core and core-half sites were 7 and 6 nucleotides in length, respectively (FIG. 6A). To generate a suitable control oligonucleotide (MUT-QRE-1 and MUT-QRE-2, SEQ ID NO:49 and SEQ ID NO:50), we introduced guanine residues in the core sites (UACGAAC) as it is well-established that the ACGAA would not serve as a binding site for QKI and does not contain a consensus motif for other established RNA-binding proteins (Ray, D. et al., 2013, Nature, 499 (7457): 172-177). To improve cellular uptake we conjugated a cholesterol moiety to the 3′ end of the QKI-inhibiting oligonucleotides (also named decoy RNAs or dcRNAs), while a DY647 conjugate was added to the 5′ end allow for visualization of oligonucleotide uptake (FIG. 6B). All residues possess an O-methyl modification of the 2′-position of the sugar moiety to limit endonuclease-mediated degradation, while phosphorothioates were incorporated at the 2 most 5′-end nucleotides and 4 most 3′-end nucleotides. The absence of phosphorothioates in the middle portion of the decoy limits chirality, as flexibility of the consensus region could impact QKI binding to the consensus sequence(s). The dcRNAs were designed with and without the 5′-DY647 conjugate for visualization (FIG. 6). As shown in FIG. 6C, treatment of HITC6 vascular smooth muscle cells with QRE-D1 (SEQ ID NO:47) and QRE-D2 (SEQ ID NO: 48) both yielded a shift towards myocardin exon 2a inclusion as compared to a untreated control (left lane) and mutated versions of the QKI-inhibitor that contained a G residue in the core site (lanes 2 and 4) (MUT-QRE-1 and MUT-QRE-2, SEQ ID NO: 49 and SEQ ID NO:50). These studies are notable in that they provide initial evidence that inhibition of QKI activity using QKI-binding site mimetics can induce a splicing shift.
Next, we conjugated biotin to the 5′-end of the dcRNAs and performed pull-down assays with streptavidin beads and Western blotting to determine the ability to bind QKI protein. As shown in FIG. 25, SEQ ID NO:74 (which contains two ACUAAC QKI binding domains, SEQ ID NO: 87) displays little ability to interact with and of the distinct QKI protein isoforms. Similarly, SEQ ID NO:62, mutated QKI binding domains (UACGAAC) separated by a 5 ribonucleic acid-containing spacer displayed little to no ability to bind QKI protein (FIG. 26). However, the incorporation of a 5 nucleobase spacer between the UACUAAC sequences (SEQ ID NO:63) results in a significant improvement in QKI binding to the dcRNA, where nuclear binding (FIG. 25A-C, left 5 lanes) and cytoplasmic binding (FIG. 25A-C, right 6 lanes) were both observed. Of note is the fact that QKI-6 (middle panel) and QKI-7 appear to interact with the dcRNA slightly more preferentially in the cytoplasmic fraction, in keeping with a primarily cytoplasmic localization for these isoforms (FIG. 25C, right lanes). Importantly, SEQ ID NO:62 (control dcRNA) and SEQ ID NO:63 (QKI-inhibiting dcRNA) represent the oligonucleotides that were employed in vivo to determine whether QKI-inhibition could protect against UUO-mediated injury of the kidney.
Subsequently, we developed additional QKI-inhibiting RNAs (dcRNAs) in efforts to establish design rules for such dcRNAs. For these studies, we elected to proceed with core sequences, consisting of ACUAAC motifs, whereby we first tested the ability of 5′-biotin conjugated oligonucleotide containing either 2, 3, 4 or 6 copies of said motif in succession to bind to QKI protein. As shown in FIG. 25 and FIG. 26, as compared to SEQ ID NO:62 or a scrambled dcRNA (termed scr: SEQ ID NO: 119: Bio-OGoCoAoAoUoCoCoGoCoAoAoUoCoCoGoCoAoAoUoCoC wherein Bio denotes C6 biotin; oA, oC, oG, oU denotes 2′-O-Methyl modified ribonucleic acids) which display little to no binding of QKI-5 protein, the introduction of additional QKI core sequences resulted in enhanced QKI binding (SEQ ID NO: 77>SEQ ID NO: 76>SEQ ID NO:75>SEQ ID NO:74).
Next, we took SEQ ID NO:76 and introduced various chemical methods of separation between the individual ACUAAC QKI-binding sequences, including 3 ribonucleic acids (SEQ ID NO:68), 3 deoxyribonucleic acids (SEQ ID NO:66), 3 locked nucleic acid-modified ribonucleic acids (SEQ ID NO: 67) or internal triethylene glycol (TEG) spacers (termed ‘spacer 9’; SEQ ID NO: 69). The incorporation of RNA, DNA and spacer 9 into the oligonucleotides did not hamper nor improve interaction with QKI-5 protein (FIG. 26). In contrast, locked nucleic acid-modified RNA incorporation into the spacer region completely blocked the ability of QKI-5 to bind the oligonucleotide. These studies provide the insight that locked nucleic acid-modified RNAs either sterically hinder QKI protein from accessing the binding motifs (Dhuri, K. et al. Journal of Clinical Medicine. 2020, 9:2004) or alter oligonucleotide structure whereby QKI interaction is prevented (Campbell, M. A & Wengel, J. 2011, Chemical Society Reviews, 40 (12), 5680-5689). As such, the binding data for QKI-inhibiting oligonucleotides support the notion that multiple QKI binding sites improve the ability of QKI to bind, while the introduction of spacing between individual QKI-binding sequences (U) ACUAAC (UACUAAC, UACUAAY and UACUAAU, wherein Y is C or U) can enhance QKI interaction.

### QKI-Inhibiting Oligonucleotides Affect Splicing of QKI-Targeted Pre-mRNAs

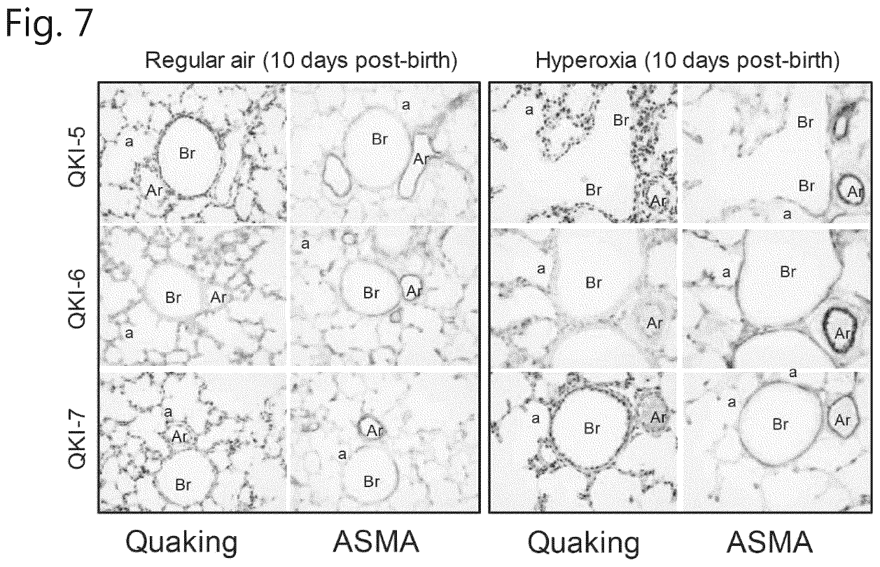

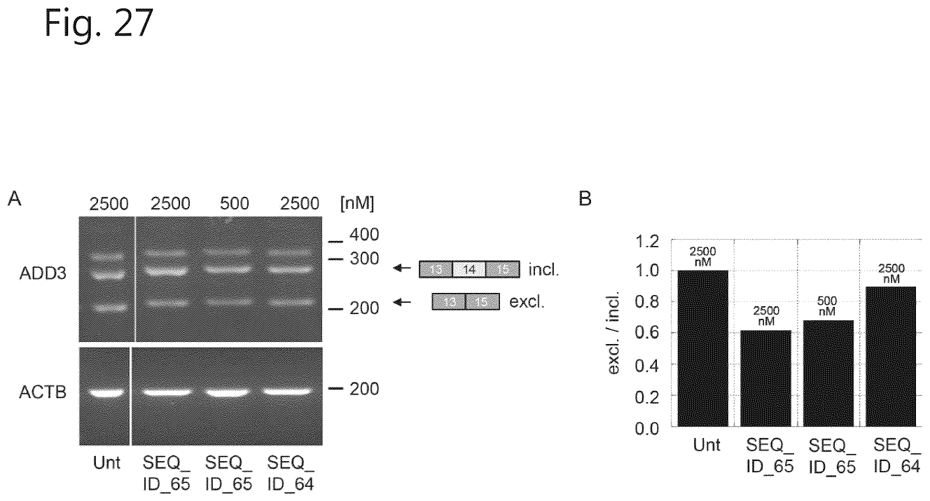

Having identified that various QKI-inhibiting oligonucleotides can bind the distinct QKI protein isoforms, we next assessed whether treatment of HEK293 cells with selected oligonucleotides could result in alteration of pre-mRNA splicing patterns for established QKI targets (de Bruin, R. G. et al. Nature Communications. 2016, 7:10846). For this, we screened SEQ ID NO:64 (mutated control) and SEQ ID NO: 65 at various concentrations as compared to a untreated control. As shown in FIG. 27, treatment of HEK293 cells with SEQ ID NO:65 increased inclusion of ADD3 exon 14 ADD3 (panel A), as evidenced by an effect that was dose-dependent (FIG. 27, panel B).
Lung Injury is Associated with QKI-Mediated Inflammation and Fibrosis
Rat pups exposed to 100% of oxygen for 10 days develop severe lung pathology with permanently enlarged alveoli due to an arrest in alveolar development and tissue damage, and an overwhelming inflammatory and fibrotic response (de Visser, Y. P. et al., 2012, American Journal of Physiology Lung Cellular Molecular Physiology, 302 (1): L56-L57; Chen, X. et al., 2017, Frontiers in Physiology, 8:486). This collective response is highly similar to bronchopulmonary dysplasia (BPD) or neonatal chronic lung disease which is observed in prematurely born infants treated with supplemental suffering for severe respiratory distress. Given that QKI protein levels were clearly increased in the setting of acute kidney injury (IRI-induced), we first sought to determine if injury to the lung would similarly impact QKI expression levels. As shown in FIG. 7, QKI is readily expressed in lung tissue of healthy newborn rats that have been exposed to regular air for the first 10 days post-birth (left panel). Exposure for 9 days to hyperoxic conditions (90% oxygen) clearly results in increased expression of the nuclear QKI-5, while QKI-6 and QKI-7 display subtle yet evident increases in expression (FIG. 7, right panel). Of note, in the lung, all three isoforms display nuclear localization, where QKI-7 in particular appears to gain cytoplasmic distribution in response to injury (FIG. 6, bottom right panel).

### Effects of QKI Inhibition on Lung Airway Development, Inflammation and Collagen Deposition

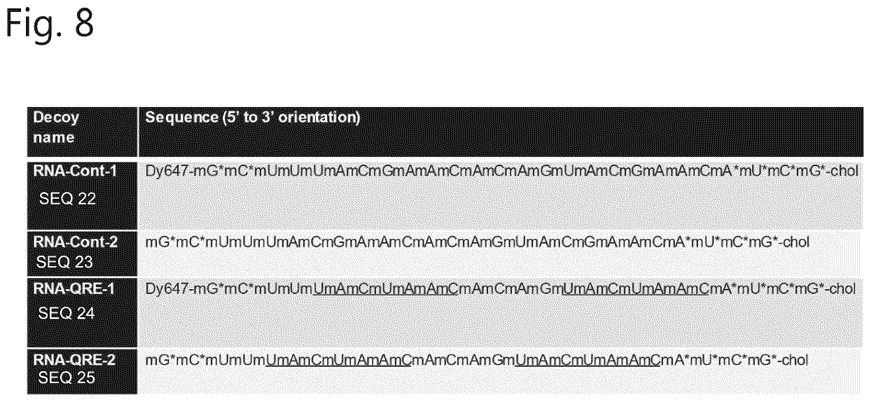

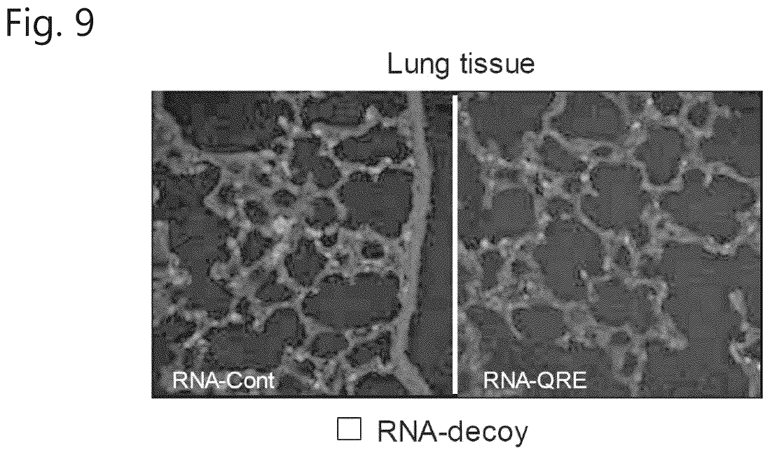

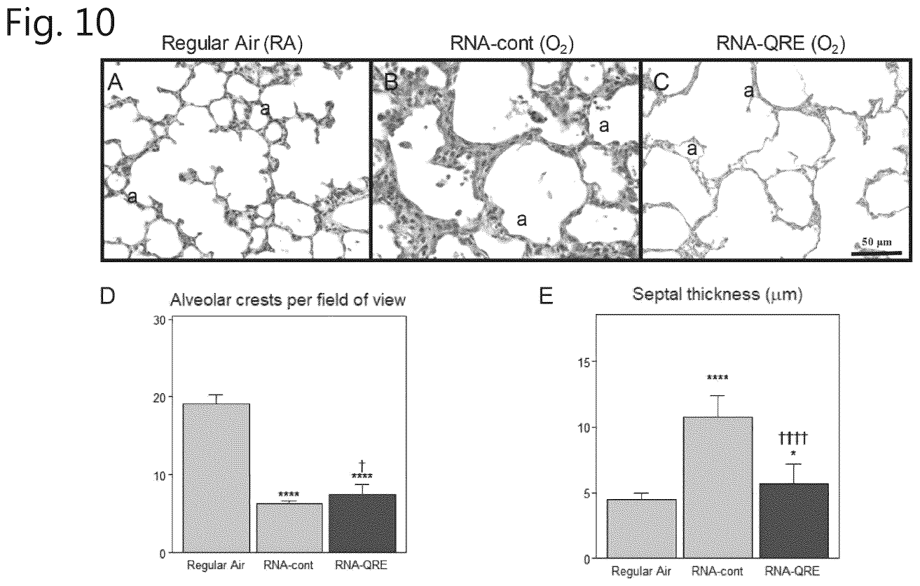

Having identified that QKI-inhibiting oligonucleotides could influence cellular splicing events that we previously have shown to be directly impacted by QKI expression levels (van der Veer, E. P. et al., 2013, Circulation Research, 113 (9): 1065-1075), we next sought to assess whether inhibition of QKI could impact the degree of lung injury in a rat model of BPD. Here, we developed a QKI-inhibiting oligonucleotide that contained a dual core element separated by 4 nucleotides. Once again, guanine residues were introduced in the core sites (UACGAAC) along with a cholesterol conjugate for improved cellular uptake and DY647 conjugate for oligonucleotide tracking in vivo (FIG. 8). All residues possess a O-Me modification of the 2′-position of the sugar moiety to limit endonuclease-mediated degradation, while phosphorothioates were incorporated at the 2 most 5′-end nucleotides and 4 most 3′-end nucleotides, while the middle portion of the oligonucleotide once again was phosphorothioate-free for maximal flexibility and ability to interact with QKI.
With these oligonucleotides we treated newborn rats with a single, subcutaneous 40 mg/kg dose of either a control oligonucleotide (RNA-Cont-1 or SEQ ID NO: 22) or the QKI-inhibiting oligonucleotide (RNA-QRE-1 or SEQ ID NO: 24) 2 days post-birth. This resulted in clear uptake of the oligonucleotides in lung tissue of the newborn rats (FIG. 9). Exposure to 100% O₂ for 10 days resulted in alveolar simplification with a heterogeneous distribution of enlarged alveoli showing a reduced number of alveolar crests (2.5-fold, p<0.001; 10B and D) surrounded by thick septa (1.7-fold, p<0.01; 9B and E) compared to RA-exposed controls. QKI inhibition did not impact the number of alveolar crests relative to RNA-Cont-1 (SEQ ID NO:22) treated rat pups (FIG. 10D). In addition, neonatal exposure to 100% O₂ induced an inflammatory and fibrotic response, characterized by a significant influx of neutrophils (25.1-fold, p<0.001; 11B-D) and macrophages (15.3-fold, p<0.001; 12B-D) and increased collagen 3A deposition in thick alveolar septa (16.6-fold, p<0.001; 13B-D), compared to RA controls. Treatment of hyperoxia-induced experimental BPD with RNA-QRE-1 (SEQ ID NO:24) reduced alveolar septal thickness (1.4-fold, p<0.01; 10C and E), the influx of neutrophils (4.2-fold, p<0.001; 11C and D) and macrophages (1.9-fold, p<0.001; 12C and D), and collagen 3A expression (1.5-fold, p<0.01; 13C and D), compared to O₂-exposed controls treated with the control RNA (RNA-Cont-1, SEQ ID NO:22).

### Effects of QKI Inhibition on Pulmonary Vascular Remodeling and Right Ventricular Hypertrophy

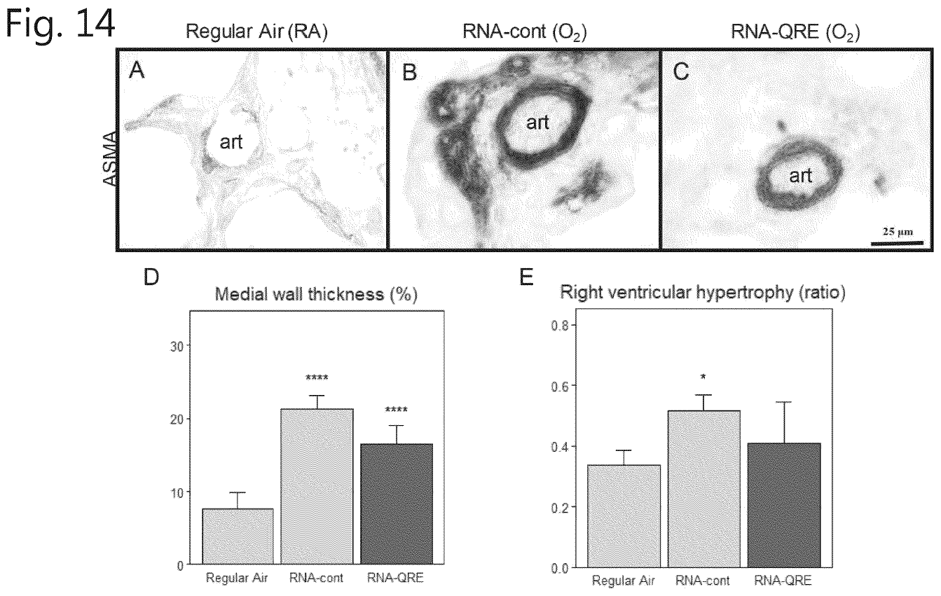

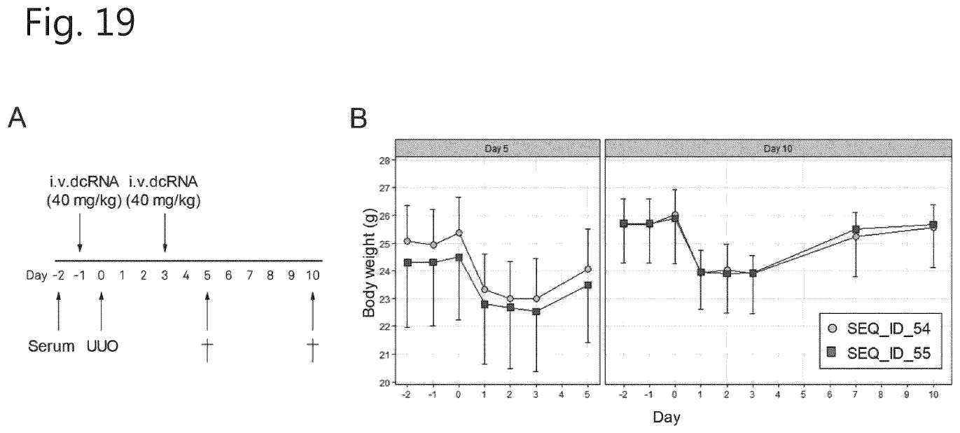

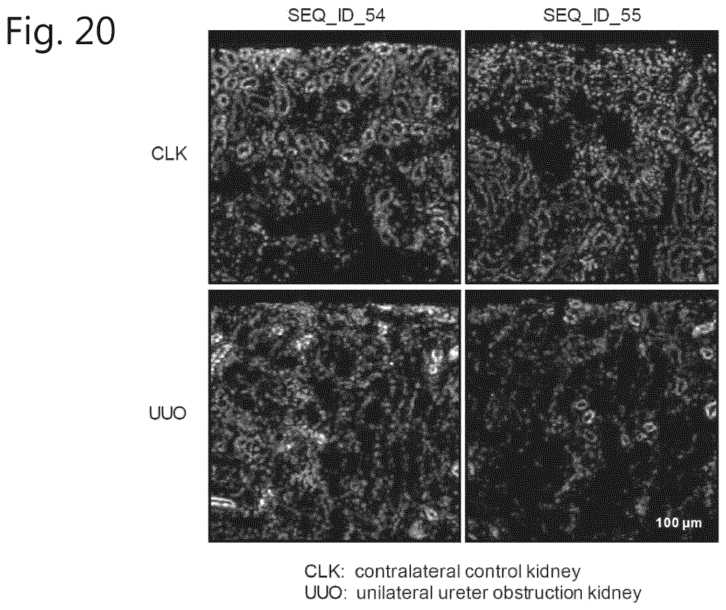

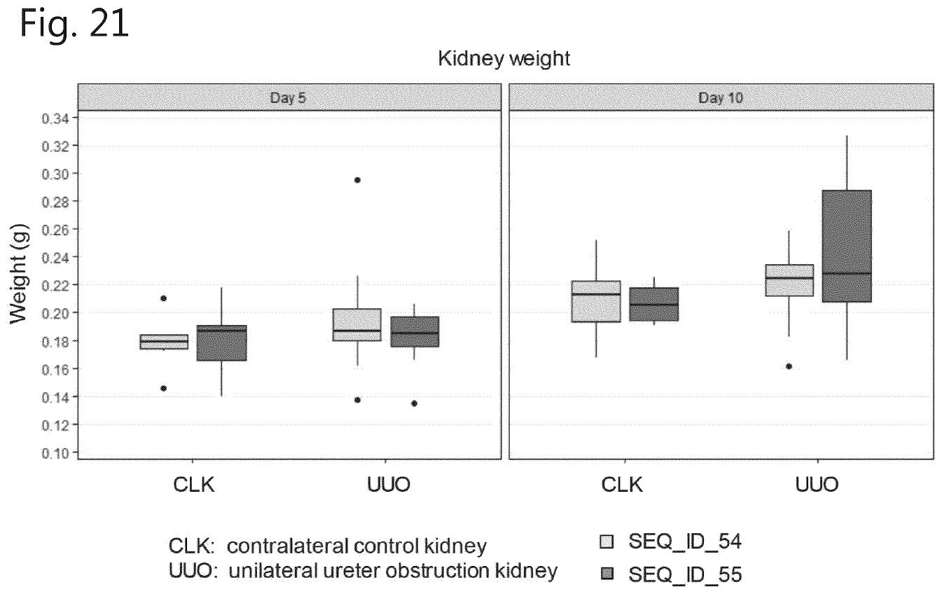

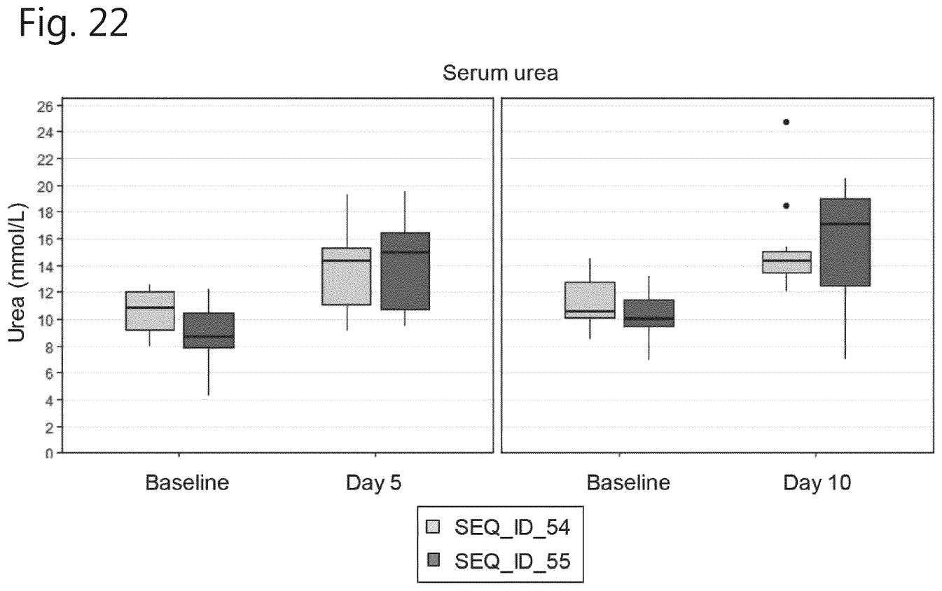

Exposure to 100% O₂ for 10 days induced vascular remodeling with increased pulmonary arterial medial wall thickness (2.2-fold, p<0.001; 13B and D), determined on ASMA-stained sections (as shown in FIG. 7), as a marker for vascular remodeling and PAH. In addition, the ratio RV/LV free wall thickness (1.2-fold, p<0.05; 5E) as a marker for RVH increased after exposure to hyperoxia. While hyperoxic conditions yielded a significant increase in right ventricular hypertrophy (RVH) in RNA-Cont-1 (SEQ ID NO:22) treated rat pups, treatment with RNA-QRE-1 (SEQ ID NO:24) did not allow RVH to progress to a similar level (FIG. 14E). However, no beneficial effect on hyperoxia-induced pulmonary vascular remodelling (14C and D) and RVH (14E) was observed between RNA-Cont-1 (SEQ ID NO:22) and RNA-QRE-1 (SEQ ID NO:24) treatment.
Kidney Distribution of Decoy RNAs does not Impact Kidney Weight and Function Following UUO
Having identified that QKI-inhibiting oligonucleotides could limit lung injury in the setting of bronchopulmonary dysplasia, we subsequently tested the ability of SEQ ID NO:55 to limit kidney inflammation and fibrosis following UUO. For this, we prophylactically administered a first intravenous dose of SEQ ID NO:55 or the control oligonucleotide SEQ ID NO:54 at 40 mg/kg into C57Bl6 mice. At 24 hours, we performed UUO by making a left flank incision and double-ligating the lower pole of the kidney with 2 separate silk ties. Subsequently, a second 40 mg/kg intravenous dose of the respective oligonucleotides was adminstered and the mice were sacrificed on days 5 and 10 post-surgery (n=11 mice per arm per day).
Over the course of the study, we tracked body weight of the mice and observed no discernable effect of SEQ ID NO:54 or SEQ ID NO:55 dosing, with the sole clear impact of body weight loss being observed in the first days post-surgery (FIG. 19). Following sacrificing, we examined the weight of the contralateral (CLK) and UUO-injured kidneys. No differences in kidney weight were detected at day 5, with day 10 UUO kidneys, although UUO kidneys treated with SEQ ID NO:55 displayed a greater range of kidney weights relative to those wherein SEQ ID NO: 54 were administered (FIG. 20). As shown in FIG. 21, immunohistochemical examination of oligonucleotide distribution using a phosphorothioate-detecting antibody revealed excellent uptake in the kidneys, with clear accumulation in the proximal tubules of the kidney cortex. Finally, serum urea was also scored to determine if oligonucleotide accumulation in proximal tubules would impact kidney function, which revealed no effects on increased urea (FIG. 22).

### Effects of QKI Inhibition on Kidney Inflammation and Collagen Deposition Following UUO

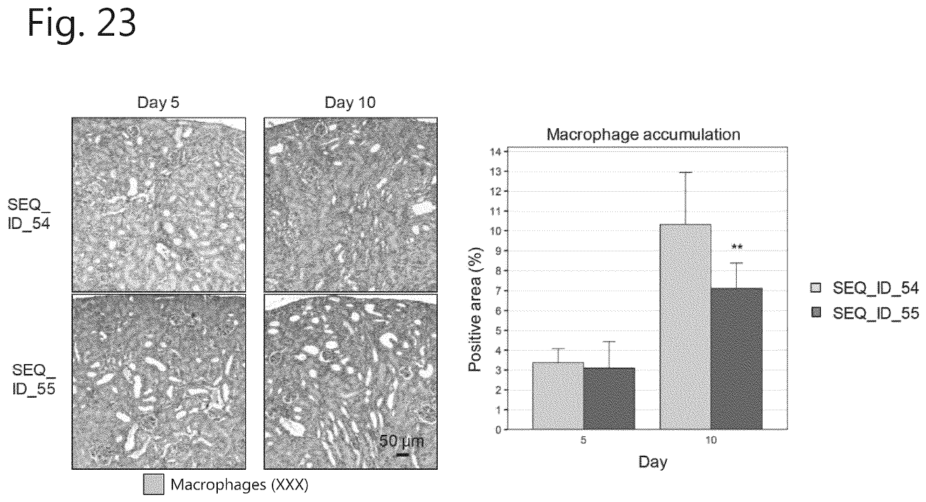

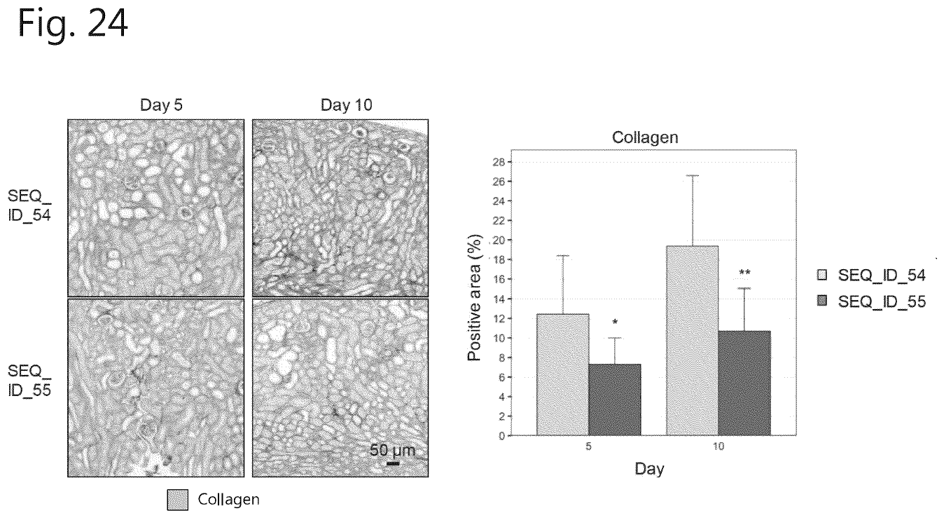

Inspection of UUO-injured mouse kidneys exposed to SEQ ID NO:54 and SEQ ID NO:55 for evidence of monocyte infiltration into the kidney and macrophage accumulation by immunohistochemical staining for F/80. As shown in FIG. 23, at day 5 no discernable difference in macrophage accumulation was observed (left panels in graph). However, 10 days post-UUO a significant reduction in macrophage accumulation was observed in UUO-injured C57Bl6 mice treated with SEQ ID NO: 55 as compared to mice exposed to SEQ ID NO:54. Next, we assessed whether the SEQ ID NO:55-mediated attenuation of macrophage numbers could also impact collagen accumulation in the kidney interstitium. As shown in FIG. 24, SEQ ID NO:55-treated mice revealed a striking reduction in collagen kidney levels at both day 5 and day 10 post-UUO. This observation is particularly relevant given that previous studies designed to assess whether decreasing QKI expression could limit macrophage accumulation as well as collagen deposition in the kidney interstitium post-UUO revealed significant attenuation of both parameters 5 days post-injury, an effect that was lost 10 days post-UUO (de Bruin, R. G. et al., 2020, Epigenomics, 4 (2)). Hence, the data presented here with SEQ ID NO:55 suggest that inhibition of RBP activity with oligonucleotides (dcRNAs) could represent a more effective means of protecting organs against injury than RBP abrogation.

1. An oligonucleotide comprising a core QKI binding site UACUAAY and optionally a half QKI binding site YAAY, wherein Y is C or U, which is able to bind a QKI protein and as a result is able to inhibit an activity of said QKI protein.

2. An oligonucleotide according to claim 1 comprising two core QKI binding sites UACUAAC and no half QKI binding site, wherein the length of the oligonucleotide is from 14 to 40 nucleotides.

3. An oligonucleotide according to claim 1 comprising one core QKI binding site UACUAAC and one half QKI binding site YAAY, wherein the length of the oligonucleotide is from 12 to 39 nucleotides.

4. An oligonucleotide according to claim 1, comprising only one QKI core binding site UACUAAY and no half QKI binding site, wherein the length of the oligonucleotide is from 7 to 22 nucleotides.

5. An oligonucleotide according to claim 1, wherein the core and the half QKI binding sites are separated by 1-20 nucleotides.

6. An oligonucleotide according to claim 1, wherein the half QKI binding site is present upstream/5′ side of the core QKI binding site or wherein the half QKI binding site is present downstream/3′ side of the core QKI binding site.

7. An oligonucleotide comprising, consisting of or consisting essentially of (ACUAAY) n wherein Y is C or U and n is an integer ranged from 1 to 6 (SEQ ID NO: 100-104 for n=2-6, respectively).

8. An oligonucleotide comprising, consisting of or consisting essentially of (UACUAAY) n wherein Y is C or U and n is an integer ranged from 1 to 6 (SEQ ID NO: 106-110 for n=2-6, respectively).

9. An oligonucleotide according to claim 1, wherein the length of such oligonucleotide is ranged from 6 to 50 nucleotides.

10. An oligonucleotide according to claim 1, wherein the oligonucleotide is conjugated to a peptide, vitamin, aptamer, carbohydrate or mixtures of carbohydrates, protein, small molecule, antibody, polymer, drug, lithocholic acid, eicosapentanoic acid or a cholesterol moiety.

11. An oligonucleotide according to claim 1, wherein a GalNac moiety has been conjugated to it's 5′ or 3′ end.

12. An oligonucleotide according to claim 1, wherein a small molecule, aptamer or antibody has been conjugated to it, either at the 5′ or 3′ end.

13. An oligonucleotide according to claim 1, which is a single stranded oligonucleotide.

14. An oligonucleotide according to claim 1, which is a modified RNA oligonucleotide comprising a nucleotide analogue and/or a modified internucleotide linkage.

15. An oligonucleotide according to claim 1, wherein the backbone of the central part of the oligonucleotide has not been modified.

16. An oligonucleotide according to claim 2, wherein the oligonucleotide is as follows:
GCUUUACUAACACAGUACUAACAUCG (SEQ ID NO:11), wherein the underlined nucleotides have a phosphorothioate linkage and all nucleotides have a 2-O′ methyl base.

17. An oligonucleotide according to claim 1, wherein the oligonucleotide comprises, consists of or essentially consists of SEQ ID NO: 55, 57, 59, 61, 63, 65, 66, 68, 69, 70, 71, 72, 73, 74, 78, 79, 81, 82, 90, 91, 92, 93, 94, 95, 96, 97, 100, 101, 102, 103, 104, 107, 108, 109,110, 111, 112, 113, 114, 115, 116, 117, 118.

18. A viral vector comprising a nucleic acid sequence encoding the oligonucleotide as defined in claim 1.

19. A composition comprising an oligonucleotide as defined in claim 1.

20. (canceled)

21. A method according to claim 23, wherein the disease or condition is an inflammatory disease or condition.

22. A method according to claim 21, wherein the oligonucleotide is able to induce a therapeutic activity, effect, result in such disease or condition.

23. A method for preventing, treating, curing, ameliorating and/or delaying a condition or disease in an individual, in a cell, tissue or organ of said individual, wherein said method comprises administering an oligonucleotide according to claim 1 to said individual or a subject in the need thereof.

24. A composition comprising a viral vector as defined in claim 18.


SEQUENCE LISTING (119 sequences)

SEQ ID NO: 1-9 - skipped (9 entries)

SEQ ID NO: 10 - RNA, 26 nt, synthetic construct
  source 1..26: mol_type = other RNA; note = Oligonucleotide (1)
gctttactaacacagtactaacatcg

SEQ ID NO: 11 - RNA, 26 nt, synthetic construct
  source 1..26: mol_type = other RNA; note = Oligonucleotide (1)
  modified_base 1..26: mod_base = OTHER; note = 2'-O-Methyl modified ribonucleic acid
  modified_base 1^2: mod_base = OTHER; note = phosphorothioate linkage
  modified_base 23..26: mod_base = OTHER; note = phosphorothioate linkage
gctttactaacacagtactaacatcg

SEQ ID NO: 12 - RNA, 29 nt, synthetic construct
  source 1..29: mol_type = other RNA; note = Oligonucleotide (2)
gctttactaacactcacctactaacatcg

SEQ ID NO: 13 - RNA, 29 nt, synthetic construct
  source 1..29: mol_type = other RNA; note = Oligonucleotide (2)
  modified_base 1..29: mod_base = OTHER; note = 2'-O-Methyl modified ribonucleic acid
  modified_base 1^2: mod_base = OTHER; note = phosphorothioate linkage
  modified_base 26..29: mod_base = OTHER; note = phosphorothioate linkage
gctttactaacactcacctactaacatcg

SEQ ID NO: 14 - RNA, 25 nt, synthetic construct
  source 1..25: mol_type = other RNA; note = Oligonucleotide (3)
gccgtaaccacgtctactaacgccg

SEQ ID NO: 15 - RNA, 25 nt, synthetic construct
  source 1..25: mol_type = other RNA; note = Oligonucleotide (3)
  modified_base 1..25: mod_base = OTHER; note = 2'-O-Methyl modified ribonucleic acid
  modified_base 1^2: mod_base = OTHER; note = phosphorothioate linkage
  modified_base 22..25: mod_base = OTHER; note = phosphorothioate linkage
gccgtaaccacgtctactaacgccg

SEQ ID NO: 16 - protein, 341 aa, synthetic construct
  source 1..341: note = QKI-5
MVGEMETKEKPKPTPDYLMQLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPALAFSLAATAQAAPRIITGPAPVLPPAALRTPTPAGPTIMPLIRQIQTAVMPNGTPHPTAAIVPPGPEAGLIYTPYEYPYTLAPATSILEYPIEPSGVLGAVATKVRRHDMRVHPYQRIVTADRAATGN

SEQ ID NO: 17 - DNA, 1026 bp, synthetic construct
  source 1..1026: mol_type = other DNA; note = QKI-5
atggtcggggaaatggaaacgaaggagaagccgaagcccaccccagattacctgatgcagctgatgaacgacaagaagctcatgagcagcctgcccaacttctgcgggatcttcaaccacctcgagcggctgctggacgaagaaattagcagagtacggaaagacatgtacaatgacacattaaatggcagtacagagaaaaggagtgcagaattgcctgatgctgtgggacctattgttcagttacaagagaaactttatgtgcctgtaaaagaatacccagattttaattttgttgggagaatccttggacctagaggacttacagccaaacaacttgaagcagaaaccggatgtaaaatcatggtccgaggcaaaggctcaatgagggataaaaaaaaggaggagcaaaatagaggcaagcccaattgggagcatctaaatgaagatttacatgtactaatcactgtggaagatgctcagaacagagcagaaatcaaattgaagagagcagttgaagaagtgaagaaattattggtacctgcagcagaaggagaagacagcctgaagaagatgcagctgatggagcttgcgattctgaatggcacctacagagatgccaacattaaatcaccagcccttgccttttctcttgcagcaacagcccaggctgctccaaggatcattactgggcctgcgccggttctcccaccagctgccctgcgtactcctacgccagctggccctaccataatgcctttgatcagacaaatacagaccgctgtcatgccaaacggaactcctcacccaactgctgcaatagttcctccagggcccgaagctggtttaatctatacaccctatgagtacccctacacattggcaccagctacatcaatccttgagtatcctattgaacctagtggtgtattaggtgcggtggctactaaagttcgaaggcacgatatgcgtgtccatccttaccaaaggattgtgaccgcagaccgagccgccaccggcaactaa

SEQ ID NO: 18 - protein, 319 aa, synthetic construct
  source 1..319: note = QKI-6
MVGEMETKEKPKPTPDYLMQLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPALAFSLAATAQAAPRIITGPAPVLPPAALRTPTPAGPTIMPLIRQIQTAVMPNGTPHPTAAIVPPGPEAGLIYTPYEYPYTLAPATSILEYPIEPSGVLGKFFSPWG

SEQ ID NO: 19 - DNA, 960 bp, synthetic construct
  source 1..960: mol_type = other DNA; note = QKI-6
atggtcggggaaatggaaacgaaggagaagccgaagcccaccccagattacctgatgcagctgatgaacgacaagaagctcatgagcagcctgcccaacttctgcgggatcttcaaccacctcgagcggctgctggacgaagaaattagcagagtacggaaagacatgtacaatgacacattaaatggcagtacagagaaaaggagtgcagaattgcctgatgctgtgggacctattgttcagttacaagagaaactttatgtgcctgtaaaagaatacccagattttaattttgttgggagaatccttggacctagaggacttacagccaaacaacttgaagcagaaaccggatgtaaaatcatggtccgaggcaaaggctcaatgagggataaaaaaaaggaggagcaaaatagaggcaagcccaattgggagcatctaaatgaagatttacatgtactaatcactgtggaagatgctcagaacagagcagaaatcaaattgaagagagcagttgaagaagtgaagaaattattggtacctgcagcagaaggagaagacagcctgaagaagatgcagctgatggagcttgcgattctgaatggcacctacagagatgccaacattaaatcaccagcccttgccttttctcttgcagcaacagcccaggctgctccaaggatcattactgggcctgcgccggttctcccaccagctgccctgcgtactcctacgccagctggccctaccataatgcctttgatcagacaaatacagaccgctgtcatgccaaacggaactcctcacccaactgctgcaatagttcctccagggcccgaagctggtttaatctatacaccctatgagtacccctacacattggcaccagctacatcaatccttgagtatcctattgaacctagtggtgtattaggtaagttcttctccccatggggttaa

SEQ ID NO: 20 - protein, 325 aa, synthetic construct
  source 1..325: note = QKI-7
MVGEMETKEKPKPTPDYLMQLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPALAFSLAATAQAAPRIITGPAPVLPPAALRTPTPAGPTIMPLIRQIQTAVMPNGTPHPTAAIVPPGPEAGLIYTPYEYPYTLAPATSILEYPIEPSGVLEWIEMPVMPDISAH

SEQ ID NO: 21 - DNA, 978 bp, synthetic construct
  source 1..978: mol_type = other DNA; note = QKI-7
atggtcggggaaatggaaacgaaggagaagccgaagcccaccccagattacctgatgcagctgatgaacgacaagaagctcatgagcagcctgcccaacttctgcgggatcttcaaccacctcgagcggctgctggacgaagaaattagcagagtacggaaagacatgtacaatgacacattaaatggcagtacagagaaaaggagtgcagaattgcctgatgctgtgggacctattgttcagttacaagagaaactttatgtgcctgtaaaagaatacccagattttaattttgttgggagaatccttggacctagaggacttacagccaaacaacttgaagcagaaaccggatgtaaaatcatggtccgaggcaaaggctcaatgagggataaaaaaaaggaggagcaaaatagaggcaagcccaattgggagcatctaaatgaagatttacatgtactaatcactgtggaagatgctcagaacagagcagaaatcaaattgaagagagcagttgaagaagtgaagaaattattggtacctgcagcagaaggagaagacagcctgaagaagatgcagctgatggagcttgcgattctgaatggcacctacagagatgccaacattaaatcaccagcccttgccttttctcttgcagcaacagcccaggctgctccaaggatcattactgggcctgcgccggttctcccaccagctgccctgcgtactcctacgccagctggccctaccataatgcctttgatcagacaaatacagaccgctgtcatgccaaacggaactcctcacccaactgctgcaatagttcctccagggcccgaagctggtttaatctatacaccctatgagtacccctacacattggcaccagctacatcaatccttgagtatcctattgaacctagtggtgtattagagtggattgaaatgccagtcatgcctgatatttcagcccattga

SEQ ID NO: 22 - RNA, 26 nt, synthetic construct
  source 1..26: mol_type = other RNA; note = RNA Cont-1
  modified_base 1: mod_base = OTHER; note = Dy647 conjugate
  modified_base 1..26: mod_base = OTHER; note = 2'-O-Methyl modified ribonucleic acid
  modified_base 26: mod_base = OTHER; note = cholesterol conjugate (attached via a phosphorothioate)
  modified_base 1..3: mod_base = OTHER; note = phosphorothioate linkage
  modified_base 23..26: mod_base = OTHER; note = phosphorothioate linkage
gctttacgaacacagtacgaacatcg

SEQ ID NO: 23 - RNA, 26 nt, synthetic construct
  source 1..26: mol_type = other RNA; note = RNA Cont-2
  modified_base 1..26: mod_base = OTHER; note = 2'-O-Methyl modified ribonucleic acid
  modified_base 26: mod_base = OTHER; note = cholesterol conjugate (attached via a phosphorothioate)
  modified_base 1..3: mod_base = OTHER; note = phosphorothioate linkage
  modified_base 23..26: mod_base = OTHER; note = phosphorothioate linkage
gctttacgaacacagtacgaacatcg

SEQ ID NO: 24 - RNA, 26 nt, synthetic construct
  source 1..26: mol_type = other RNA; note = RNA QRE-1
  modified_base 1: mod_base = OTHER; note = Dy647 conjugate
  modified_base 1..26: mod_base = OTHER; note = 2'-O-Methyl modified ribonucleic acid
  modified_base 26: mod_base = OTHER; note = cholesterol conjugate (attached via a phosphorothioate)
  modified_base 1..3: mod_base = OTHER; note = phosphorothioate linkage
  modified_base 23..26: mod_base = OTHER; note = phosphorothioate linkage
gctttactaacacagtactaacatcg

SEQ ID NO: 25 - RNA, 26 nt, synthetic construct
  source 1..26: mol_type = other RNA; note = RNA QRE-2
  modified_base 1..26: mod_base = OTHER; note = 2'-O-Methyl modified ribonucleic acid
  modified_base 26: mod_base = OTHER; note = cholesterol conjugate (attached via a phosphorothioate)
  modified_base 1..3: mod_base = OTHER; note = phosphorothioate linkage
  modified_base 23..26: mod_base = OTHER; note = phosphorothioate linkage
gctttactaacacagtactaacatcg

SEQ ID NO: 26 - DNA, 19 bp, synthetic construct
  source 1..19: mol_type = other DNA; note = Primer
ttccaggagcgagatccct

SEQ ID NO: 27 - DNA, 20 bp, synthetic construct
  source 1..20: mol_type = other DNA; note = Primer
actcccactcttccaccttc

SEQ ID NO: 28 - DNA, 19 bp, synthetic construct
  source 1..19: mol_type = other DNA; note = Primer
cacccatgacgaacatggg

SEQ ID NO: 29 - DNA, 19 bp, synthetic construct
  source 1..19: mol_type = other DNA; note = Primer
caccaccctgttgctgtag

SEQ ID NO: 30 - DNA, 19 bp, synthetic construct
  source 1..19: mol_type = other DNA; note = Primer
ctgtcatgccaaacggaac

SEQ ID NO: 31 - DNA, 20 bp, synthetic construct
  source 1..20: mol_type = other DNA; note = Primer
cccagtggtgtgttaggtgc

SEQ ID NO: 32 - DNA, 19 bp, synthetic construct
  source 1..19: mol_type = other DNA; note = Primer
gatggacacgcatatcgtg

SEQ ID NO: 33 - DNA, 21 bp, synthetic construct
  source 1..21: mol_type = other DNA; note = Primer
cctttggtaaggatggacacg

SEQ ID NO: 34 - DNA, 19 bp, synthetic construct
  source 1..19: mol_type = other DNA; note = Primer
ctgtcatgccaaacggaac

SEQ ID NO: 35 - DNA, 24 bp, synthetic construct
  source 1..24: mol_type = other DNA; note = Primer
aagttactggtacctgcggctgaa

SEQ ID NO: 36 - DNA, 17 bp, synthetic construct
  source 1..17: mol_type = other DNA; note = Primer
cgttgggaaagccatac

SEQ ID NO: 37 - DNA, 24 bp, synthetic construct
  source 1..24: mol_type = other DNA; note = Primer
gaaagccataccctaacaccactg

SEQ ID NO: 38 - DNA, 19 bp, synthetic construct
  source 1..19: mol_type = other DNA; note = Primer
ctgtcatgccaaacggaac

SEQ ID NO: 39 - DNA, 24 bp, synthetic construct
  source 1..24: mol_type = other DNA; note = Primer
aagttactggtacctgcggctgaa

SEQ ID NO: 40 - DNA, 19 bp, synthetic construct
  source 1..19: mol_type = other DNA; note = Primer
gactggcatttcaatccac

SEQ ID NO: 41 - DNA, 26 bp, synthetic construct
  source 1..26: mol_type = other DNA; note = Primer
gcatgactggcatttcaatccactct

SEQ ID NO: 42 - DNA, 20 bp, synthetic construct
  source 1..20: mol_type = other DNA; note = Primer
gtgctggactctggagatgg

SEQ ID NO: 43 - DNA, 20 bp, synthetic construct
  source 1..20: mol_type = other DNA; note = Primer
cgtggctattccttcgtgac

SEQ ID NO: 44 - DNA, 20 bp, synthetic construct
  source 1..20: mol_type = other DNA; note = Primer
aatagccacgctcagtcagg

SEQ ID NO: 45 - DNA, 20 bp, synthetic construct
  source 1..20: mol_type = other DNA; note = Primer
gcagttcgtagctcttctcc

SEQ ID NO: 46 - skipped

SEQ ID NO: 47 - RNA, 29 nt, synthetic construct
  source 1..29: mol_type = other RNA; note = QRE-D1
  modified_base 1: mod_base = OTHER; note = Dy647 conjugate
  modified_base 1..29: mod_base = OTHER; note = 2'-O-Methyl modified ribonucleic acid
  modified_base 29: mod_base = OTHER; note = cholesterol conjugate (attached via a phosphorothioate)
  modified_base 1..3: mod_base = OTHER; note = phosphorothioate linkage
  modified_base 26..29: mod_base = OTHER; note = phosphorothioate linkage
gctttactaacactcacctactaacatcg

SEQ ID NO: 48 - RNA, 25 nt, synthetic construct
  source 1..25: mol_type = other RNA; note = QRE-D2
  modified_base 1: mod_base = OTHER; note = Dy647 conjugate
  modified_base 1..25: mod_base = OTHER; note = 2'-O-Methyl modified ribonucleic acid
  modified_base 25: mod_base = OTHER; note = cholesterol conjugate (attached via a phosphorothioate)
  modified_base 1..3: mod_base = OTHER; note = phosphorothioate linkage
  modified_base 22..25: mod_base = OTHER; note = phosphorothioate linkage
gccgtaaccacgtctactaacgccg

SEQ ID NO: 49 - RNA, 29 nt, synthetic construct
  source 1..29: mol_type = other RNA; note = MUT-QRE-1
  modified_base 1: mod_base = OTHER; note = Dy647 conjugate
  modified_base 1..29: mod_base = OTHER; note = 2'-O-Methyl modified ribonucleic acid
  modified_base 29: mod_base = OTHER; note = cholesterol conjugate (attached via a phosphorothioate)
  modified_base 1..3: mod_base = OTHER; note = phosphorothioate linkage
  modified_base 26..29: mod_base = OTHER; note = phosphorothioate linkage
gctttacgaacactcacctactaacatcg

SEQ ID NO: 50 - RNA, 25 nt, synthetic construct
  source 1..25: mol_type = other RNA; note = MUT-QRE-2
  modified_base 1: mod_base = OTHER; note = Dy647 conjugate
  modified_base 1..25: mod_base = OTHER; note = 2'-O-Methyl modified ribonucleic acid
  modified_base 25: mod_base = OTHER; note = cholesterol conjugate (attached via a phosphorothioate)
  modified_base 1..3: mod_base = OTHER; note = phosphorothioate linkage
  modified_base 22..25: mod_base = OTHER; note = phosphorothioate linkage
gccgtaaccacgtctacgaacgccg

SEQ ID NO: 51 - RNA, 14 nt, synthetic construct
  source 1..14: mol_type = other RNA; note = 2 core QKI binding sites
tactaactactaac

SEQ ID NO: 52 - RNA, 14 nt, synthetic construct
  source 1..14: mol_type = other RNA; note = 2 core QKI binding sites
tactaaytactaay

SEQ ID NO: 53 - RNA, 14 nt, synthetic construct
  source 1..14: mol_type = other RNA; note = 2 core QKI binding sites
tactaattactaat

SEQ ID NO: 54 - RNA, 26 nt, synthetic construct
  source 1..26: mol_type = other RNA; note = oligonucleotide
gctttacgaacacagtacgaacatcg

SEQ ID NO: 55 - RNA, 26 nt, synthetic construct
  source 1..26: mol_type = other RNA; note = oligonucleotide
gctttactaacacagtactaacatcg

SEQ ID NO: 56 - RNA, 29 nt, synthetic construct
  source 1..29: mol_type = other RNA; note = oligonucleotide
gctttacgaacactcacctactaacatcg

SEQ ID NO: 57 - RNA, 29 nt, synthetic construct
  source 1..29: mol_type = other RNA; note = oligonucleotide
gctttactaacactcacctactaacatcg

SEQ ID NO: 58 - RNA, 25 nt, synthetic construct
  source 1..25: mol_type = other RNA; note = oligonucleotide
gccgtaaccacgtctacgaacgccg

SEQ ID NO: 59 - RNA, 25 nt, synthetic construct
  source 1..25: mol_type = other RNA; note = oligonucleotide
gccgtaaccacgtctactaacgccg

SEQ ID NO: 60 - RNA, 27 nt, synthetic construct
  source 1..27: mol_type = other RNA; note = oligonucleotide
gctttacgaacacagatacgaacatcg

SEQ ID NO: 61 - RNA, 27 nt, synthetic construct
  source 1..27: mol_type = other RNA; note = oligonucleotide
gctttactaacacagatactaacatcg

SEQ ID NO: 62 - RNA, 27 nt, synthetic construct
  source 1..27: mol_type = other RNA; note = oligonucleotide
  modified_base 1: mod_base = OTHER; note = C6 biotin conjugate
  modified_base 1..27: mod_base = OTHER; note = 2'-O-Methyl modified ribonucleic acid
gctttacgaacacagatacgaacatcg

SEQ ID NO: 63 - RNA, 27 nt, synthetic construct
  source 1..27: mol_type = other RNA; note = oligonucleotide
  modified_base 1: mod_base = OTHER; note = C6 biotin conjugate
  modified_base 1..27: mod_base = OTHER; note = 2'-O-Methyl modified ribonucleic acid
gctttactaacacagatactaacatcg

SEQ ID NO: 64 - RNA, 27 nt, synthetic construct
  source 1..27: mol_type = other RNA; note = oligonucleotide
  modified_base 1..27: mod_base = OTHER; note = 2'-O-Methyl modified ribonucleic acid
  modified_base 1..27: mod_base = OTHER; note = phosphorothioate linkage
gctttacgaacacagatacgaacatcg

SEQ ID NO: 65 - RNA, 27 nt, synthetic construct
  source 1..27: mol_type = other RNA; note = oligonucleotide
  modified_base 1..27: mod_base = OTHER; note = 2'-O-Methyl modified ribonucleic acid
  modified_base 1..27: mod_base = OTHER; note = phosphorothioate linkage
gctttactaacacagatactaacatcg

SEQ ID NO: 66 - RNA, 33 nt, synthetic construct
  source 1..33: mol_type = other RNA; note = oligonucleotide
  modified_base 1: mod_base = OTHER; note = C6 biotin conjugate
  modified_base 1..6: mod_base = OTHER; note = 2'-O-Methyl modified ribonucleic acid
  modified_base 10..15: mod_base = OTHER; note = 2'-O-Methyl modified ribonucleic acid
  modified_base 19..24: mod_base = OTHER; note = 2'-O-Methyl modified ribonucleic acid
  modified_base 28..33: mod_base = OTHER; note = 2'-O-Methyl modified ribonucleic acid
  modified_base 7..9: mod_base = OTHER; note = deoxyribonucleic acid
  modified_base 16..18: mod_base = OTHER; note = deoxyribonucleic acid
  modified_base 25..27: mod_base = OTHER; note = deoxyribonucleic acid
  modified_base 7..9: mod_base = OTHER; note = 5-methyluridine
  modified_base 16..18: mod_base = OTHER; note = 5-methyluridine
  modified_base 25..27: mod_base = OTHER; note = 5-methyluridine
actaactttactaactttactaactttactaac

SEQ ID NO: 67 - RNA, 33 nt, synthetic construct; identical to SEQ ID NO: 66
  source 1..33: mol_type = other RNA; note = oligonucleotide
  modified_base 1: mod_base = OTHER; note = C6 biotin conjugate
  modified_base 1..6: mod_base = OTHER; note = 2'-O-Methyl modified ribonucleic acid
  modified_base 10..15: mod_base = OTHER; note = 2'-O-Methyl modified ribonucleic acid
  modified_base 19..24: mod_base = OTHER; note = 2'-O-Methyl modified ribonucleic acid
  modified_base 28..33: mod_base = OTHER; note = 2'-O-Methyl modified ribonucleic acid
  modified_base 7..9: mod_base = OTHER; note = locked nucleic acid
  modified_base 16..18: mod_base = OTHER; note = locked nucleic acid
  modified_base 25..27: mod_base = OTHER; note = locked nucleic acid
  modified_base 7..9: mod_base = OTHER; note = 5-methyluridine
  modified_base 16..18: mod_base = OTHER; note = 5-methyluridine
  modified_base 25..27: mod_base = OTHER; note = 5-methyluridine

SEQ ID NO: 68 - RNA, 33 nt, synthetic construct; identical to SEQ ID NO: 66
  source 1..33: mol_type = other RNA; note = oligonucleotide
  modified_base 1: mod_base = OTHER; note = C6 biotin conjugate
  modified_base 1..33: mod_base = OTHER; note = 2'-O-Methyl modified ribonucleic acid

SEQ ID NO: 69 - RNA, 24 nt, synthetic construct
  source 1..24: mol_type = other RNA; note = oligonucleotide
  modified_base 1: mod_base = OTHER; note = C6 biotin conjugate
  modified_base 1..24: mod_base = OTHER; note = 2'-O-Methyl modified ribonucleic acid
  modified_base 6^7: mod_base = OTHER; note = 9-triethylene glycol (TEG) spacer
  modified_base 12^13: mod_base = OTHER; note = 9-triethylene glycol (TEG) spacer
  modified_base 18^19: mod_base = OTHER; note = 9-triethylene glycol (TEG) spacer
actaacactaacactaacactaac

SEQ ID NO: 70 - RNA, 12 nt, synthetic construct
  source 1..12: mol_type = other RNA; note = core QKI binding site
actaacactaac

SEQ ID NO: 71 - RNA, 18 nt, synthetic construct
  source 1..18: mol_type = other RNA; note = core QKI binding site
actaacactaacactaac

SEQ ID NO: 72 - RNA, 24 nt, synthetic construct
  source 1..24: mol_type = other RNA; note = core QKI binding site
actaacactaacactaacactaac

SEQ ID NO: 73 - RNA, 36 nt, synthetic construct
  source 1..36: mol_type = other RNA; note = core QKI binding site
actaacactaacactaacactaacactaacactaac

SEQ ID NO: 74 - RNA, 12 nt, synthetic construct
  source 1..12: mol_type = other RNA; note = oligonucleotide
  modified_base 1: mod_base = OTHER; note = C6 biotin conjugate
  modified_base 1..12: mod_base = OTHER; note = 2'-O-Methyl modified ribonucleic acid
actaacactaac

SEQ ID NO: 75 - RNA, 18 nt, synthetic construct
  source 1..18: mol_type = other RNA; note = oligonucleotide
  modified_base 1: mod_base = OTHER; note = C6 biotin conjugate
  modified_base 1..18: mod_base = OTHER; note = 2'-O-Methyl modified ribonucleic acid
actaacactaacactaac

SEQ ID NO: 76 - RNA, 24 nt, synthetic construct
  source 1..24: mol_type = other RNA; note = oligonucleotide
  modified_base 1: mod_base = OTHER; note = C6 biotin conjugate
  modified_base 1..24: mod_base = OTHER; note = 2'-O-Methyl modified ribonucleic acid
actaacactaacactaacactaac

SEQ ID NO: 77 - RNA, 36 nt, synthetic construct; identical to SEQ ID NO: 73
  source 1..36: mol_type = other RNA; note = oligonucleotide
  modified_base 1: mod_base = OTHER; note = C6 biotin conjugate
  modified_base 1..36: mod_base = OTHER; note = 2'-O-Methyl modified ribonucleic acid

SEQ ID NO: 78 - RNA, 24 nt, synthetic construct
  source 1..24: mol_type = other RNA; note = oligonucleotide
  modified_base 1: mod_base = OTHER; note = C6 biotin conjugate
actaacactaacactaacactaac

SEQ ID NO: 79 - RNA, 24 nt, synthetic construct
  source 1..24: mol_type = other RNA; note = oligonucleotide
  modified_base 1: mod_base = OTHER; note = C6 biotin conjugate
  modified_base 1..24: mod_base = OTHER; note = phosphorothioate linkage
actaacactaacactaacactaac

SEQ ID NO: 80 - RNA, 18 nt, synthetic construct
  source 1..18: mol_type = other RNA; note = oligonucleotide
  modified_base 1..18: mod_base = OTHER; note = 2'-O-Methyl modified ribonucleic acid
actaacactaacactaac

SEQ ID NO: 81 - RNA, 24 nt, synthetic construct
  source 1..24: mol_type = other RNA; note = oligonucleotide
  modified_base 1..18: mod_base = OTHER; note = 2'-O-Methyl modified ribonucleic acid
  modified_base 20..24: mod_base = OTHER; note = 2'-O-Methyl modified ribonucleic acid
actaacactaacactaacactaac

SEQ ID NO: 82 - RNA, 24 nt, synthetic construct
  source 1..24: mol_type = other RNA; note = oligonucleotide
  modified_base 1..24: mod_base = OTHER; note = 2'-O-Methyl modified ribonucleic acid
  modified_base 1..24: mod_base = OTHER; note = phosphorothioate linkage
actaacactaacactaacactaac

SEQ ID NO: 83 - DNA, 20 bp, synthetic construct
  source 1..20: mol_type = other DNA; note = Primer
accagctcctcctaacccat

SEQ ID NO: 84 - DNA, 21 bp, synthetic construct
  source 1..21: mol_type = other DNA; note = Primer
tcactcgcttagcaagctcat

SEQ ID NO: 85 - DNA, 20 bp, synthetic construct
  source 1..20: mol_type = other DNA; note = Primer
tgcgtgacattaaggagaag

SEQ ID NO: 86 - DNA, 20 bp, synthetic construct
  source 1..20: mol_type = other DNA; note = Primer
tgaaggtagtttcgtggatg

SEQ ID NO: 87-89 - skipped (3 entries)

SEQ ID NO: 90 - RNA, 12 nt, synthetic construct
  source 1..12: mol_type = other RNA; note = oligonucleotide
  modified_base 1..12: mod_base = OTHER; note = 2'-O-Methyl modified ribonucleic acid
actaacactaac

SEQ ID NO: 91 - RNA, 18 nt, synthetic construct
  source 1..18: mol_type = other RNA; note = oligonucleotide
  modified_base 1..18: mod_base = OTHER; note = 2'-O-Methyl modified ribonucleic acid
actaacactaacactaac

SEQ ID NO: 92 - RNA, 24 nt, synthetic construct
  source 1..24: mol_type = other RNA; note = oligonucleotide
  modified_base 1..24: mod_base = OTHER; note = 2'-O-Methyl modified ribonucleic acid
actaacactaacactaacactaac

SEQ ID NO: 93 - RNA, 36 nt, synthetic construct; identical to SEQ ID NO: 73
  source 1..36: mol_type = other RNA; note = oligonucleotide
  modified_base 1..36: mod_base = OTHER; note = 2'-O-Methyl modified ribonucleic acid

SEQ ID NO: 94 - RNA, 14 nt, synthetic construct
  source 1..14: mol_type = other RNA; note = core QKI binding site
tactaactactaac

SEQ ID NO: 95 - RNA, 21 nt, synthetic construct
  source 1..21: mol_type = other RNA; note = core QKI binding site
tactaactactaactactaac

SEQ ID NO: 96 - RNA, 28 nt, synthetic construct
  source 1..28: mol_type = other RNA; note = core QKI binding site
tactaactactaactactaactactaac

SEQ ID NO: 97 - RNA, 42 nt, synthetic construct
  source 1..42: mol_type = other RNA; note = core QKI binding site
tactaactactaactactaactactaactactaactactaac

SEQ ID NO: 98-99 - skipped (2 entries)

SEQ ID NO: 100 - RNA, 12 nt, synthetic construct
  source 1..12: mol_type = other RNA; note = core QKI binding site
actaayactaay

SEQ ID NO: 101 - RNA, 18 nt, synthetic construct
  source 1..18: mol_type = other RNA; note = core QKI binding site
actaayactaayactaay

SEQ ID NO: 102 - RNA, 24 nt, synthetic construct
  source 1..24: mol_type = other RNA; note = core QKI binding site
actaayactaayactaayactaay

SEQ ID NO: 103 - RNA, 30 nt, synthetic construct
  source 1..30: mol_type = other RNA; note = core QKI binding site
actaayactaayactaayactaayactaay

SEQ ID NO: 104 - RNA, 36 nt, synthetic construct
  source 1..36: mol_type = other RNA; note = core QKI binding site
actaayactaayactaayactaayactaayactaay

SEQ ID NO: 105 - skipped

SEQ ID NO: 106 - RNA, 14 nt, synthetic construct
  source 1..14: mol_type = other RNA; note = core QKI binding site
tactaaytactaay

SEQ ID NO: 107 - RNA, 21 nt, synthetic construct
  source 1..21: mol_type = other RNA; note = core QKI binding site
tactaaytactaaytactaay

SEQ ID NO: 108 - RNA, 28 nt, synthetic construct
  source 1..28: mol_type = other RNA; note = core QKI binding site
tactaaytactaaytactaaytactaay

SEQ ID NO: 109 - RNA, 35 nt, synthetic construct
  source 1..35: mol_type = other RNA; note = core QKI binding site
tactaaytactaaytactaaytactaaytactaay

SEQ ID NO: 110 - RNA, 42 nt, synthetic construct
  source 1..42: mol_type = other RNA; note = core QKI binding site
tactaaytactaaytactaaytactaaytactaaytactaay

SEQ ID NO: 111 - RNA, 33 nt, synthetic construct; identical to SEQ ID NO: 66
  source 1..33: mol_type = other RNA; note = oligonucleotide
  modified_base 1..33: mod_base = OTHER; note = 2'-O-Methyl modified ribonucleic acid

SEQ ID NO: 112 - RNA, 33 nt, synthetic construct; identical to SEQ ID NO: 66
  source 1..33: mol_type = other RNA; note = oligonucleotide
  modified_base 1..6: mod_base = OTHER; note = 2'-O-Methyl modified ribonucleic acid
  modified_base 10..15: mod_base = OTHER; note = 2'-O-Methyl modified ribonucleic acid
  modified_base 19..24: mod_base = OTHER; note = 2'-O-Methyl modified ribonucleic acid
  modified_base 28..33: mod_base = OTHER; note = 2'-O-Methyl modified ribonucleic acid
  modified_base 7..9: mod_base = OTHER; note = deoxyribonucleic acid
  modified_base 16..18: mod_base = OTHER; note = deoxyribonucleic acid
  modified_base 25..27: mod_base = OTHER; note = deoxyribonucleic acid
  modified_base 7..9: mod_base = OTHER; note = 5-methyluridine
  modified_base 16..18: mod_base = OTHER; note = 5-methyluridine
  modified_base 25..27: mod_base = OTHER; note = 5-methyluridine

SEQ ID NO: 113 - RNA, 24 nt, synthetic construct
  source 1..24: mol_type = other RNA; note = oligonucleotide
  modified_base 1..24: mod_base = OTHER; note = 2'-O-Methyl modified ribonucleic acid
  modified_base 6^7: mod_base = OTHER; note = 9-triethylene glycol (TEG) spacer
  modified_base 12^13: mod_base = OTHER; note = 9-triethylene glycol (TEG) spacer
  modified_base 18^19: mod_base = OTHER; note = 9-triethylene glycol (TEG) spacer
actaacactaacactaacactaac

SEQ ID NO: 114 - RNA, 24 nt, synthetic construct
  source 1..24: mol_type = other RNA; note = oligonucleotide
  modified_base 1..24: mod_base = OTHER; note = 2'-O-Methyl modified ribonucleic acid
  modified_base 6^7: mod_base = OTHER; note = 9-triethylene glycol (TEG) spacer
  modified_base 12^13: mod_base = OTHER; note = 9-triethylene glycol (TEG) spacer
  modified_base 18^19: mod_base = OTHER; note = 9-triethylene glycol (TEG) spacer
actaacactaacactaacactaac

SEQ ID NO: 115 - RNA, 29 nt, synthetic construct
  source 1..29: mol_type = other RNA; note = QRE-D1 (without Dy647, cholesterol attached via a phosphorothioate)
  modified_base 1..29: mod_base = OTHER; note = 2'-O-Methyl modified ribonucleic acid
  modified_base 29: mod_base = OTHER; note = cholesterol conjugate (attached via a phosphorothioate)
  modified_base 1..3: mod_base = OTHER; note = phosphorothioate linkage
  modified_base 26..29: mod_base = OTHER; note = phosphorothioate linkage
gctttactaacactcacctactaacatcg

SEQ ID NO: 116 - RNA, 25 nt, synthetic construct
  source 1..25: mol_type = other RNA; note = QRE-D2 (without Dy647, with cholesterol attached via a phosphorothioate)
  modified_base 1..25: mod_base = OTHER; note = 2'-O-Methyl modified ribonucleic acid
  modified_base 25: mod_base = OTHER; note = cholesterol conjugate (attached via a phosphorothioate)
  modified_base 1..3: mod_base = OTHER; note = phosphorothioate linkage
  modified_base 22..25: mod_base = OTHER; note = phosphorothioate linkage
gccgtaaccacgtctactaacgccg

SEQ ID NO: 117 - RNA, 29 nt, synthetic construct
  source 1..29: mol_type = other RNA; note = QRE-D1 (without Dy647 and cholesterol)
  modified_base 1..29: mod_base = OTHER; note = 2'-O-Methyl modified ribonucleic acid
  modified_base 1..3: mod_base = OTHER; note = phosphorothioate linkage
  modified_base 26..29: mod_base = OTHER; note = phosphorothioate linkage
gctttactaacactcacctactaacatcg

SEQ ID NO: 118 - RNA, 25 nt, synthetic construct
  source 1..25: mol_type = other RNA; note = QRE-D2 (without Dy647 and cholesterol)
  modified_base 1..25: mod_base = OTHER; note = 2'-O-Methyl modified ribonucleic acid
  modified_base 1..3: mod_base = OTHER; note = phosphorothioate linkage
  modified_base 22..25: mod_base = OTHER; note = phosphorothioate linkage
gccgtaaccacgtctactaacgccg

SEQ ID NO: 119 - RNA, 21 nt, synthetic construct
  source 1..21: mol_type = other RNA; note = Scrambled control
  modified_base 1: mod_base = OTHER; note = C6 biotin conjugate
  modified_base 1..21: mod_base = OTHER; note = 2'-O-Methyl modified ribonucleic acid
gcaatccgcaatccgcaatcc